SEQUENCE LISTING (827 sequences)

SEQ ID NO: 1 - protein, 8 aa, synthetic construct
GFTFRDYS

SEQ ID NO: 2 - protein, 8 aa, synthetic construct
IRFDGTTK

SEQ ID NO: 3 - protein, 11 aa, synthetic construct
VKDNGWRAFDH

SEQ ID NO: 4 - protein, 11 aa, synthetic construct
QSLLHRNGYIY

SEQ ID NO: 5 - skipped

SEQ ID NO: 6 - protein, 9 aa, synthetic construct
MQSLENPWT

SEQ ID NO: 7 - protein, 114 aa, synthetic construct
VESGGGVVQPGASLRLSCVASGFTFRDYSMHWVRQAPGKGLEWMSFIRFDGTTKDYRESVKGRFIISRDDSKNTLYLQMTSLRPEDTARYYCVKDNGWRAFDHWGQGALVIVSS

SEQ ID NO: 8 - protein, 105 aa, synthetic construct
EIVMTQSPLSLPVTPGEAASISCRSSQSLLHRNGYIYLDWYLQRPGQSPQLLISFVSKRASGAPDRFSGSGSGTDFTLTISRVEAEDFGVYFCMQSLENPWTFGP

SEQ ID NO: 9 - protein, 21 aa, synthetic construct
VESGGGVVQPGASLRLSCVAS

SEQ ID NO: 10 - protein, 17 aa, synthetic construct
MHWVRQAPGKGLEWMSF

SEQ ID NO: 11 - protein, 38 aa, synthetic construct
DYRESVKGRFIISRDDSKNTLYLQMTSLRPEDTARYYC

SEQ ID NO: 12 - protein, 11 aa, synthetic construct
WGQGALVIVSS

SEQ ID NO: 13 - protein, 26 aa, synthetic construct
EIVMTQSPLSLPVTPGEAASISCRSS

SEQ ID NO: 14 - protein, 17 aa, synthetic construct
LDWYLQRPGQSPQLLIS

SEQ ID NO: 15 - protein, 36 aa, synthetic construct
KRASGAPDRFSGSGSGTDFTLTISRVEAEDFGVYFC

SEQ ID NO: 16 - skipped

SEQ ID NO: 17 - protein, 441 aa, synthetic construct
  VARIANT 138: note = misc_feature - Xaa can be any naturally occurring amino acid
  VARIANT 208: note = misc_feature - Xaa can be any naturally occurring amino acid
  VARIANT 352: note = misc_feature - Xaa can be any naturally occurring amino acid
VESGGGVVQPGASLRLSCVASGFTFRDYSMHWVRQAPGKGLEWMSFIRFDGTTKDYRESVKGRFIISRDDSKNTLYLQMTSLRPEDTARYYCVKDNGWRAFDHWGQGALVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 209 aa, synthetic construct
EIVMTQSPLSLPVTPGEAASISCRSSQSLLHRNGYIYLDWYLQRPGQSPQLLISFVSKRASGAPDRFSGSGSGTDFTLTISRVEAEDFGVYFCMQSLENPWTFGPAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 19 - DNA, 344 bp, synthetic construct
  source 1..344: mol_type = other DNA
ggtggagtcggggggcggcgtggtccagcctggggcgtccctgagactctcctgtgtggcgtctggattcacctttcgtgactattccatgcactgggtccgccaggctccaggcaaggggctggagtggatgtcatttatacggtttgatgggaccactaaggactatcgagagtctgtgaagggccgattcatcatttccagagacgactccaagaacacactgtatctgcaaatgacgagcctgagacctgaggacacggctcgatattactgtgtgaaagacaatgggtggcgggcctttgaccactggggccagggagccctggtcatcgtctcctcag

SEQ ID NO: 20 - DNA, 316 bp, synthetic construct
  source 1..316: mol_type = other DNA
gagattgtgatgactcagtctccactctccctgcccgtcacccctggagaggcggcctccatctcctgcaggtctagtcagagcctcctccatcgtaatggatacatctacttggactggtacctgcagaggccagggcagtcaccacaactcctgatctctttcgtttctaaacgggcctccggggcccctgacaggttcagtggctctggttcaggcacagatttcacactgacaattagtagagtggaggctgaggactttggggtttatttctgcatgcaatcgctagaaaacccctggacgttcggcccag

SEQ ID NO: 21 - protein, 8 aa, synthetic construct
GFTFRDYS

SEQ ID NO: 22 - protein, 8 aa, synthetic construct
IRFDGTTK

SEQ ID NO: 23 - protein, 11 aa, synthetic construct
AKDNGWRAFDH

SEQ ID NO: 24 - protein, 11 aa, synthetic construct
QSLLHRNGYIY

SEQ ID NO: 25 - skipped

SEQ ID NO: 26 - protein, 9 aa, synthetic construct
MQALETPWT

SEQ ID NO: 27 - protein, 111 aa, synthetic construct
GGGVVQPGTSLRLSCVASGFTFRDYSMHWVRQAPGKGLEWVSFIRFDGTTKDYRDSVKGRFIISRDDSKNTLYLQMTSLRVEDTALYYCAKDNGWRAFDHWGQGALVTVSS

SEQ ID NO: 28 - protein, 110 aa, synthetic construct
VMTQSPLSLPVTPGEAASISCRSSQSLLHRNGYIYLDWYLQRPGQSPQLLISFVSKRASGAPDRFSGSGSGTDFTLTISRVEAEDFGVYFCMQALETPWTFGPGTKLEIK

SEQ ID NO: 29 - protein, 18 aa, synthetic construct
GGGVVQPGTSLRLSCVAS

SEQ ID NO: 30 - protein, 17 aa, synthetic construct
MHWVRQAPGKGLEWVSF

SEQ ID NO: 31 - protein, 38 aa, synthetic construct
DYRDSVKGRFIISRDDSKNTLYLQMTSLRVEDTALYYC

SEQ ID NO: 32 - protein, 11 aa, synthetic construct
WGQGALVTVSS

SEQ ID NO: 33 - protein, 24 aa, synthetic construct
VMTQSPLSLPVTPGEAASISCRSS

SEQ ID NO: 34 - protein, 17 aa, synthetic construct
LDWYLQRPGQSPQLLIS

SEQ ID NO: 35 - protein, 36 aa, synthetic construct; identical to SEQ ID NO: 15

SEQ ID NO: 36 - protein, 10 aa, synthetic construct
FGPGTKLEIK

SEQ ID NO: 37 - protein, 438 aa, synthetic construct
  VARIANT 135: note = misc_feature - Xaa can be any naturally occurring amino acid
  VARIANT 205: note = misc_feature - Xaa can be any naturally occurring amino acid
  VARIANT 349: note = misc_feature - Xaa can be any naturally occurring amino acid
GGGVVQPGTSLRLSCVASGFTFRDYSMHWVRQAPGKGLEWVSFIRFDGTTKDYRDSVKGRFIISRDDSKNTLYLQMTSLRVEDTALYYCAKDNGWRAFDHWGQGALVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
VMTQSPLSLPVTPGEAASISCRSSQSLLHRNGYIYLDWYLQRPGQSPQLLISFVSKRASGAPDRFSGSGSGTDFTLTISRVEAEDFGVYFCMQALETPWTFGPGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 39 - DNA, 336 bp, synthetic construct
  source 1..336: mol_type = other DNA
cggggggcggcgtggtccagcctgggacgtccctgagactctcctgtgtagcgtctggattcaccttccgtgactattccatgcactgggtccgccaggctccaggcaaggggctggagtgggtgtcatttatacggtttgatgggaccactaaggactatcgagactctgtgaagggccgattcatcatctccagagacgactccaagaacacactgtatctgcagatgacgagcctgagagttgaggacacggctttatattactgtgcgaaagacaatgggtggcgggcctttgaccactggggccagggagccctggtcaccgtctcctcag

SEQ ID NO: 40 - DNA, 331 bp, synthetic construct
  source 1..331: mol_type = other DNA
gtgatgactcagtctccactctccctgcccgtcacccctggagaggcggcctccatctcctgcaggtctagtcagagcctcctccatcgtaatggatacatctacttggattggtacctgcagaggccagggcagtctccacaactcctgatctctttcgtttctaaacgggcctccggggcccctgacaggttcagtggctctggttcaggcacagattttacactgacaatcagcagagtggaggctgaggattttggggtttatttctgcatgcaagcgctagaaaccccctggacgttcggcccagggaccaaactggagatcaaac

SEQ ID NO: 41 - protein, 8 aa, synthetic construct
GDSISSYY

SEQ ID NO: 42 - protein, 7 aa, synthetic construct
IFTSGST

SEQ ID NO: 43 - protein, 19 aa, synthetic construct
ARDRRALSSDGNWYWYFDL

SEQ ID NO: 44 - protein, 6 aa, synthetic construct
QTITRN

SEQ ID NO: 45 - skipped

SEQ ID NO: 46 - protein, 9 aa, synthetic construct
QQSDNTPRT

SEQ ID NO: 47 - protein, 120 aa, synthetic construct
ESGPRLVKPSETLSLTCIVSGDSISSYYWGWIRQPAGRGLEWIGRIFTSGSTTYNPSLKSRVSMSVETSKNQFSLTLTSVTAADTAVYFCARDRRALSSDGNWYWYFDLWGRGTLVAVSS

SEQ ID NO: 48 - protein, 103 aa, synthetic construct
TQSPSSLSASVGDRVTITCRASQTITRNLNWYQQKSGEAPKLLIYGASILQSGVPSRFTGSGSGTDFTLTISNLQPEDFASYSCQQSDNTPRTFGQGTKVEIK

SEQ ID NO: 49 - protein, 20 aa, synthetic construct
ESGPRLVKPSETLSLTCIVS

SEQ ID NO: 50 - protein, 17 aa, synthetic construct
WGWIRQPAGRGLEWIGR

SEQ ID NO: 51 - protein, 38 aa, synthetic construct
TYNPSLKSRVSMSVETSKNQFSLTLTSVTAADTAVYFC

SEQ ID NO: 52 - protein, 11 aa, synthetic construct
WGRGTLVAVSS

SEQ ID NO: 53 - protein, 22 aa, synthetic construct
TQSPSSLSASVGDRVTITCRAS

SEQ ID NO: 54 - protein, 17 aa, synthetic construct
LNWYQQKSGEAPKLLIY

SEQ ID NO: 55 - protein, 36 aa, synthetic construct
ILQSGVPSRFTGSGSGTDFTLTISNLQPEDFASYSC

SEQ ID NO: 56 - protein, 10 aa, synthetic construct
FGQGTKVEIK

SEQ ID NO: 57 - protein, 447 aa, synthetic construct
  VARIANT 144: note = misc_feature - Xaa can be any naturally occurring amino acid
  VARIANT 214: note = misc_feature - Xaa can be any naturally occurring amino acid
  VARIANT 358: note = misc_feature - Xaa can be any naturally occurring amino acid
ESGPRLVKPSETLSLTCIVSGDSISSYYWGWIRQPAGRGLEWIGRIFTSGSTTYNPSLKSRVSMSVETSKNQFSLTLTSVTAADTAVYFCARDRRALSSDGNWYWYFDLWGRGTLVAVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 207 aa, synthetic construct
TQSPSSLSASVGDRVTITCRASQTITRNLNWYQQKSGEAPKLLIYGASILQSGVPSRFTGSGSGTDFTLTISNLQPEDFASYSCQQSDNTPRTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 59 - DNA, 362 bp, synthetic construct
  source 1..362: mol_type = other DNA
ggagtcgggcccacgactggtgaagccttcggagaccctgtccctcacctgcattgtctctggtgactccatcagtagttattattggggctggatccggcagcccgccgggaggggactggagtggattgggcggatctttaccagcgggagcaccacctataatccctccctcaagagtcgagtctccatgtcagtagagacatccaagaaccagttctccctgacactgacctctgtgaccgccgcggacacggccgtttatttctgtgcgagagatcgaagggcactttcgtctgacggcaactggtactggtacttcgatctctggggccgtggcaccctggtcgctgtctcctcgg

SEQ ID NO: 60 - DNA, 311 bp, synthetic construct
  source 1..311: mol_type = other DNA
gacccagtctccatcctccctgtctgcatctgtcggagacagagtcaccatcacttgccgggcaagtcagaccattactcggaatttaaattggtatcagcagaaatcaggggaagcccctaagctcctgatctatggtgcatccattttgcaaagtggggtcccatcaaggttcactggcagtggatctgggacagatttcactctcaccatcagtaatctgcaacctgaagattttgcaagttactcctgtcaacagagtgacaataccccgcggacgttcggccaagggaccaaggtggagatcaaac

SEQ ID NO: 61 - protein, 8 aa, synthetic construct
GGSMSSYY

SEQ ID NO: 62 - protein, 7 aa, synthetic construct
IFTTGST

SEQ ID NO: 63 - protein, 19 aa, synthetic construct
VRDRRGRSHDSNWYWYFDL

SEQ ID NO: 64 - protein, 6 aa, synthetic construct
QTLSRN

SEQ ID NO: 65 - skipped

SEQ ID NO: 66 - protein, 9 aa, synthetic construct
QQSDNTPRT

SEQ ID NO: 67 - protein, 125 aa, synthetic construct
QVQLQESGPGLVKPSETLSLTCTVSGGSMSSYYWGWIRQPAGRGLEWIGRIFTTGSTIYNASLNSRVSMSVDTSKNQFSLKLTSVTAADTALYFCVRDRRGRSHDSNWYWYFDLWGRGTLVTVSS

SEQ ID NO: 68 - protein, 85 aa, synthetic construct
RVIITCRASQTLSRNLNWYQQKPGEAPKLLIYGASTLQSGVPSRFTGSGSGTDFTLIISGLQPEDFATYYCQQSDNTPRTFGQGT

SEQ ID NO: 69 - protein, 25 aa, synthetic construct
QVQLQESGPGLVKPSETLSLTCTVS

SEQ ID NO: 70 - protein, 17 aa, synthetic construct
WGWIRQPAGRGLEWIGR

SEQ ID NO: 71 - protein, 38 aa, synthetic construct
IYNASLNSRVSMSVDTSKNQFSLKLTSVTAADTALYFC

SEQ ID NO: 72 - protein, 11 aa, synthetic construct
WGRGTLVTVSS

SEQ ID NO: 73 - protein, 9 aa, synthetic construct
RVIITCRAS

SEQ ID NO: 74 - protein, 17 aa, synthetic construct
LNWYQQKPGEAPKLLIY

SEQ ID NO: 75 - protein, 36 aa, synthetic construct
TLQSGVPSRFTGSGSGTDFTLIISGLQPEDFATYYC

SEQ ID NO: 76 - protein, 5 aa, synthetic construct
FGQGT

SEQ ID NO: 77 - protein, 452 aa, synthetic construct
  VARIANT 149: note = misc_feature - Xaa can be any naturally occurring amino acid
  VARIANT 219: note = misc_feature - Xaa can be any naturally occurring amino acid
  VARIANT 363: note = misc_feature - Xaa can be any naturally occurring amino acid
QVQLQESGPGLVKPSETLSLTCTVSGGSMSSYYWGWIRQPAGRGLEWIGRIFTTGSTIYNASLNSRVSMSVDTSKNQFSLKLTSVTAADTALYFCVRDRRGRSHDSNWYWYFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 189 aa, synthetic construct
RVIITCRASQTLSRNLNWYQQKPGEAPKLLIYGASTLQSGVPSRFTGSGSGTDFTLIISGLQPEDFATYYCQQSDNTPRTFGQGTAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 79 - DNA, 376 bp, synthetic construct
  source 1..376: mol_type = other DNA
caggtgcagctgcaggagtcgggcccaggactggtgaagccttcggagaccctgtccctcacctgcactgtctctggtggctccatgagtagttactactggggctggatccggcagcccgccgggaggggactggagtggattgggcgaatcttcaccactgggagcaccatctacaacgcctccctcaacagtcgagtctccatgtcagtagacacgtccaagaatcagttctccctgaaactgacctctgtgaccgccgcggacacggccttgtatttctgtgtgagagatcgaagagggcgatcgcatgacagcaactggtactggtacttcgatctctggggccgtggcaccctggtcactgtctcctcgg

SEQ ID NO: 80 - DNA, 258 bp, synthetic construct
  source 1..258: mol_type = other DNA
cagagtcatcatcacttgccgggcaagtcagacccttagccgcaatttaaattggtatcagcagaaaccaggggaagcccctaaactcctgatctatggtgcatccactttacaaagtggggtcccatcaaggttcactggcagtgggtctgggacagatttcactctcatcattagtggtctgcaacctgaagattttgcaacttactactgtcagcagagtgacaataccccgcggacgttcggccaagggaccaa

SEQ ID NO: 81 - protein, 8 aa, synthetic construct
GFTFSTFS

SEQ ID NO: 82 - protein, 8 aa, synthetic construct
IKPNGNDK

SEQ ID NO: 83 - protein, 16 aa, synthetic construct
ARPRGYSSTLYRYFDY

SEQ ID NO: 84 - protein, 7 aa, synthetic construct
QSVISTY

SEQ ID NO: 85 - skipped

SEQ ID NO: 86 - protein, 9 aa, synthetic construct
QQYGSSHFT

SEQ ID NO: 87 - protein, 122 aa, synthetic construct
  VARIANT 2: note = misc_feature - Xaa can be any naturally occurring amino acid
CXLVESGGGLVQPGGSLRLSCAASGFTFSTFSMSWVRQAPGKGLEWLANIKPNGNDKDYVDSVRGQFTISRDNAKNSLFLQMNNLRAEDTAVYYCARPRGYSSTLYRYFDYWGQGTLVTVSS

SEQ ID NO: 88 - protein, 108 aa, synthetic construct
  VARIANT 4..5: note = misc_feature - Xaa can be any naturally occurring amino acid
KLGXXQSPGTLSLSPGERVTLSCRASQSVISTYLAWYQQKPGQSPRLLIYGASSRATGIPDRFSGSGSGTDFTLTISRLEPEDFAVYYCQQYGSSHFTFGPGTKVDIR

SEQ ID NO: 89 - protein, 24 aa, synthetic construct
  VARIANT 2: note = misc_feature - Xaa can be any naturally occurring amino acid
CXLVESGGGLVQPGGSLRLSCAAS

SEQ ID NO: 90 - protein, 17 aa, synthetic construct
MSWVRQAPGKGLEWLAN

SEQ ID NO: 91 - protein, 38 aa, synthetic construct
DYVDSVRGQFTISRDNAKNSLFLQMNNLRAEDTAVYYC

SEQ ID NO: 92 - protein, 11 aa, synthetic construct
WGQGTLVTVSS

SEQ ID NO: 93 - protein, 26 aa, synthetic construct
  VARIANT 4..5: note = misc_feature - Xaa can be any naturally occurring amino acid
KLGXXQSPGTLSLSPGERVTLSCRAS

SEQ ID NO: 94 - protein, 17 aa, synthetic construct
LAWYQQKPGQSPRLLIY

SEQ ID NO: 95 - protein, 36 aa, synthetic construct [sequence omitted]

SEQ ID NO: 96 - protein, 10 aa, synthetic construct
FGPGTKVDIR

SEQ ID NO: 97 - protein, 449 aa, synthetic construct
  VARIANT 2: note = misc_feature - Xaa can be any naturally occurring amino acid
  VARIANT 146: note = misc_feature - Xaa can be any naturally occurring amino acid
  VARIANT 216: note = misc_feature - Xaa can be any naturally occurring amino acid
  VARIANT 360: note = misc_feature - Xaa can be any naturally occurring amino acid
CXLVESGGGLVQPGGSLRLSCAASGFTFSTFSMSWVRQAPGKGLEWLANIKPNGNDKDYVDSVRGQFTISRDNAKNSLFLQMNNLRAEDTAVYYCARPRGYSSTLYRYFDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 212 aa, synthetic construct
  VARIANT 4..5: note = misc_feature - Xaa can be any naturally occurring amino acid
KLGXXQSPGTLSLSPGERVTLSCRASQSVISTYLAWYQQKPGQSPRLLIYGASSRATGIPDRFSGSGSGTDFTLTISRLEPEDFAVYYCQQYGSSHFTFGPGTKVDIRAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 99 - DNA, 367 bp, synthetic construct
  misc_difference 5..6: note = misc_feature - n is a, c, g, or t
  source 1..367: mol_type = other DNA
tgcannctggtggagtctgggggaggcttggtccagcctgggggatccctgagactctcctgtgcagcctctggattcacctttagtaccttttcaatgagctgggtccgccaggctccagggaaggggctggagtggctggccaacataaagccaaatggaaatgacaaagactatgtggactctgtgaggggccaattcaccatctccagagacaacgccaagaactcactgtttctgcaaatgaacaacctgagagccgaggacacggccgtatattactgtgcgagaccgcgtggttatagcagcaccttgtaccgttactttgactactggggccagggaaccctggtcaccgtctcctcag

SEQ ID NO: 100 - DNA, 325 bp, synthetic construct
  misc_difference 9..12: note = misc_feature - n is a, c, g, or t
  misc_difference 14: note = misc_feature - n is a, c, g, or t
  source 1..325: mol_type = other DNA
aaattgggnnnnangcagtctccaggcaccctgtctttgtctccaggggaaagagtcaccctctcctgcagggccagtcagagtgttatcagcacttatttagcctggtaccagcagaaacctggccagtctcccaggctcctcatctatggtgcatctagtagggccactggcatcccagacaggttcagtggcagtgggtctgggacagacttcactctcaccatcagcagactggagcctgaagattttgcagtatattactgtcagcagtatggtagctcacacttcactttcggccctgggaccaaagtggatatcagac

SEQ ID NO: 101 - protein, 8 aa, synthetic construct
GFTFSSYS

SEQ ID NO: 102 - protein, 8 aa, synthetic construct
ISSSSSTI

SEQ ID NO: 103 - protein, 17 aa, synthetic construct
AREDSGRFGDIYYGMDV

SEQ ID NO: 104 - protein, 6 aa, synthetic construct
QDISNY

SEQ ID NO: 105 - skipped

SEQ ID NO: 106 - protein, 9 aa, synthetic construct
QQYDNLPYT

SEQ ID NO: 107 - protein, 123 aa, synthetic construct
CRLVESGGGLVQPGGSLRLSCAASGFTFSSYSMNWVRQAPGKGLEWVSYISSSSSTIYYADSVKGRFTISRDNAKNSLYLQMNSLRDEDTAVYYCAREDSGRFGDIYYGMDVWGQGTTVTVSS

SEQ ID NO: 108 - protein, 102 aa, synthetic construct
QSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 109 - protein, 24 aa, synthetic construct
CRLVESGGGLVQPGGSLRLSCAAS

SEQ ID NO: 110 - protein, 17 aa, synthetic construct
MNWVRQAPGKGLEWVSY

SEQ ID NO: 111 - protein, 38 aa, synthetic construct
YYADSVKGRFTISRDNAKNSLYLQMNSLRDEDTAVYYC

SEQ ID NO: 112 - protein, 11 aa, synthetic construct
WGQGTTVTVSS

SEQ ID NO: 113 - protein, 21 aa, synthetic construct
QSPSSLSASVGDRVTITCQAS

SEQ ID NO: 114 - protein, 17 aa, synthetic construct
LNWYQQKPGKAPKLLIY

SEQ ID NO: 115 - protein, 36 aa, synthetic construct
NLETGVPSRFSGSGSGTDFTFTISSLQPEDIATYYC

SEQ ID NO: 116 - protein, 10 aa, synthetic construct
FGQGTKLEIK

SEQ ID NO: 117 - protein, 450 aa, synthetic construct
  VARIANT 147: note = misc_feature - Xaa can be any naturally occurring amino acid
  VARIANT 217: note = misc_feature - Xaa can be any naturally occurring amino acid
  VARIANT 361: note = misc_feature - Xaa can be any naturally occurring amino acid
CRLVESGGGLVQPGGSLRLSCAASGFTFSSYSMNWVRQAPGKGLEWVSYISSSSSTIYYADSVKGRFTISRDNAKNSLYLQMNSLRDEDTAVYYCAREDSGRFGDIYYGMDVWGQGTTVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 206 aa, synthetic construct
QSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 119 - DNA, 370 bp, synthetic construct
  source 1..370: mol_type = other DNA
gtgcaggctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagacgaggacacggctgtgtattactgtgcgagagaggatagcggtaggttcggggatatatactacggtatggacgtctggggccaagggaccacggtcaccgtctcctca

SEQ ID NO: 120 - DNA, 308 bp, synthetic construct
  source 1..308: mol_type = other DNA
ccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgtacacttttggccaggggaccaagctggagatcaaac

SEQ ID NO: 121 - protein, 8 aa, synthetic construct
GDSISSYY

SEQ ID NO: 122 - protein, 7 aa, synthetic construct
IFTTGST

SEQ ID NO: 123 - protein, 19 aa, synthetic construct
ARDRRGLSHGTNWHWFFDL

SEQ ID NO: 124 - protein, 6 aa, synthetic construct
QTISSS

SEQ ID NO: 125 - skipped

SEQ ID NO: 126 - protein, 9 aa, synthetic construct
QQTFSPPRT

SEQ ID NO: 127 - protein, 124 aa, synthetic construct
  VARIANT 2..3: note = misc_feature - Xaa can be any naturally occurring amino acid
  VARIANT 5..6: note = misc_feature - Xaa can be any naturally occurring amino acid
VXXQXXGPGLVKPSEALSLTCSVSGDSISSYYWSWIRQSAGKGPEWIGRIFTTGSTIYNPSLKTRVSMSIDTSKNRFSLQLSSVTAADTAVYYCARDRRGLSHGTNWHWFFDLWGRGTLVTVSS

SEQ ID NO: 128 - protein, 103 aa, synthetic construct
  VARIANT 1: note = misc_feature - Xaa can be any naturally occurring amino acid
XQSPSSLSASVGDRVTITCRASQTISSSLNWYQQTPGKPPKLLIYGVSTLQTGVPSRFTGGGSGTNFNLTISSVQPEDFTTYYCQQTFSPPRTFGQGTKVEMK

SEQ ID NO: 129 - protein, 24 aa, synthetic construct
  VARIANT 2..3: note = misc_feature - Xaa can be any naturally occurring amino acid
  VARIANT 5..6: note = misc_feature - Xaa can be any naturally occurring amino acid
VXXQXXGPGLVKPSEALSLTCSVS

SEQ ID NO: 130 - protein, 17 aa, synthetic construct
WSWIRQSAGKGPEWIGR

SEQ ID NO: 131 - protein, 38 aa, synthetic construct
IYNPSLKTRVSMSIDTSKNRFSLQLSSVTAADTAVYYC

SEQ ID NO: 132 - protein, 11 aa, synthetic construct
WGRGTLVTVSS

SEQ ID NO: 133 - protein, 22 aa, synthetic construct
  VARIANT 1: note = misc_feature - Xaa can be any naturally occurring amino acid
XQSPSSLSASVGDRVTITCRAS

SEQ ID NO: 134 - protein, 17 aa, synthetic construct
LNWYQQTPGKPPKLLIY

SEQ ID NO: 135 - protein, 36 aa, synthetic construct
TLQTGVPSRFTGGGSGTNFNLTISSVQPEDFTTYYC

SEQ ID NO: 136 - protein, 10 aa, synthetic construct
FGQGTKVEMK

SEQ ID NO: 137 - protein, 451 aa, synthetic construct
  VARIANT 2..3: note = misc_feature - Xaa can be any naturally occurring amino acid
  VARIANT 5..6: note = misc_feature - Xaa can be any naturally occurring amino acid
  VARIANT 148: note = misc_feature - Xaa can be any naturally occurring amino acid
  VARIANT 218: note = misc_feature - Xaa can be any naturally occurring amino acid
  VARIANT 362: note = misc_feature - Xaa can be any naturally occurring amino acid
VXXQXXGPGLVKPSEALSLTCSVSGDSISSYYWSWIRQSAGKGPEWIGRIFTTGSTIYNPSLKTRVSMSIDTSKNRFSLQLSSVTAADTAVYYCARDRRGLSHGTNWHWFFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 207 aa, synthetic construct
  VARIANT 1: note = misc_feature - Xaa can be any naturally occurring amino acid
XQSPSSLSASVGDRVTITCRASQTISSSLNWYQQTPGKPPKLLIYGVSTLQTGVPSRFTGGGSGTNFNLTISSVQPEDFTTYYCQQTFSPPRTFGQGTKVEMKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 139 - DNA, 374 bp, synthetic construct
  misc_difference 7..10: note = misc_feature - n is a, c, g, or t
  misc_difference 16: note = misc_feature - n is a, c, g, or t
  misc_difference 18: note = misc_feature - n is a, c, g, or t
  source 1..374: mol_type = other DNA
ggtgcannnncaggantngggcccaggactggtgaagccttcggaggccctgtccctcacctgcagtgtctctggtgactccatcagtagttactattggagttggatccggcagtccgccgggaagggaccggagtggattggccgtatctttaccactgggagcaccatctacaatccatccctcaagactcgagtcagcatgtcgatagacacgtccaagaaccggttttccctgcagttgagttctgtgaccgccgcggacacggccgtatattattgtgcgagagatcgaagggggctttcgcatggcaccaattggcattggttcttcgatttatggggccgcggcaccctggtcactgtctcctcag

SEQ ID NO: 140 - DNA, 310 bp, synthetic construct
  misc_difference 2: note = misc_feature - n is a, c, g, or t
  source 1..310: mol_type = other DNA
anccagtctccatcgtccctgtctgcatctgtaggagacagagtcaccatcacttgtcgggcaagtcagaccattagcagcagtttaaattggtatcagcagacaccagggaaaccccctaaactcctgatctatggtgtatccactttgcaaactggggtcccatcaaggttcactggcggtggatctgggacaaatttcaatctcaccatcagtagtgtgcaacctgaagattttacaacttactactgtcaacagactttttctcccccgcggacttttggccaagggaccaaggtggaaatgaaac

SEQ ID NO: 141 - protein, 8 aa, synthetic construct
GDSISSYY

SEQ ID NO: 142 - protein, 7 aa, synthetic construct
IFTSGST

SEQ ID NO: 143 - protein, 19 aa, synthetic construct
ARDRRGRSHDSNWYWYFDL

SEQ ID NO: 144 - protein, 6 aa, synthetic construct
QSISTN

SEQ ID NO: 145 - skipped

SEQ ID NO: 146 - protein, 9 aa, synthetic construct
QQSDNTPRT

SEQ ID NO: 147 - protein, 121 aa, synthetic construct
QESGPGLVKPSETLSLTCTVSGDSISSYYWGWIRQPAGGGLEWIGRIFTSGSTNYNPSLKSRLTMSVDTSKNQFSLTLNSLTAADTAVYFCARDRRGRSHDSNWYWYFDLWGRGALVTVSS

SEQ ID NO: 148 - protein, 102 aa, synthetic construct
  VARIANT 55: note = misc_feature - Xaa can be any naturally occurring amino acid
QSPSSLSASVGDRVTITCRPSQSISTNLNWYQQKPGKAPKLLIYGASILQSGDPXRFTGSRSGSDFTLTISGLQPEDFATYYCQQSDNTPRTFGQGTKVETK

SEQ ID NO: 149 - protein, 21 aa, synthetic construct
QESGPGLVKPSETLSLTCTVS

SEQ ID NO: 150 - protein, 17 aa, synthetic construct
WGWIRQPAGGGLEWIGR

SEQ ID NO: 151 - protein, 38 aa, synthetic construct
NYNPSLKSRLTMSVDTSKNQFSLTLNSLTAADTAVYFC

SEQ ID NO: 152 - protein, 11 aa, synthetic construct
WGRGALVTVSS

SEQ ID NO: 153 - protein, 21 aa, synthetic construct
QSPSSLSASVGDRVTITCRPS

SEQ ID NO: 154 - protein, 17 aa, synthetic construct
LNWYQQKPGKAPKLLIY

SEQ ID NO: 155 - protein, 36 aa, synthetic construct
  VARIANT 8: note = misc_feature - Xaa can be any naturally occurring amino acid
ILQSGDPXRFTGSRSGSDFTLTISGLQPEDFATYYC

SEQ ID NO: 156 - protein, 10 aa, synthetic construct
FGQGTKVETK

SEQ ID NO: 157 - protein, 448 aa, synthetic construct
  VARIANT 145: note = misc_feature - Xaa can be any naturally occurring amino acid
  VARIANT 215: note = misc_feature - Xaa can be any naturally occurring amino acid
  VARIANT 359: note = misc_feature - Xaa can be any naturally occurring amino acid
QESGPGLVKPSETLSLTCTVSGDSISSYYWGWIRQPAGGGLEWIGRIFTSGSTNYNPSLKSRLTMSVDTSKNQFSLTLNSLTAADTAVYFCARDRRGRSHDSNWYWYFDLWGRGALVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 206 aa, synthetic construct
  VARIANT 55: note = misc_feature - Xaa can be any naturally occurring amino acid
QSPSSLSASVGDRVTITCRPSQSISTNLNWYQQKPGKAPKLLIYGASILQSGDPXRFTGSRSGSDFTLTISGLQPEDFATYYCQQSDNTPRTFGQGTKVETKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 159 - DNA, 365 bp, synthetic construct
  source 1..365: mol_type = other DNA
gcaggagtcgggcccaggactggtgaagccctcggagaccctgtccctcacctgcactgtctctggtgactccatcagtagttactattggggctggatccggcagcccgccggggggggactggagtggattgggcggatctttaccagtgggagcaccaactacaatccctccctcaagagtcggctcaccatgtcagtcgacacgtccaagaaccagttctccctgacactcaactcactgaccgccgcggacacggccgtgtatttctgtgcgagagatcgcagggggcgttcgcatgacagcaactggtactggtacttcgatctctggggccgtggcgccctggtcactgtctcctcgg

SEQ ID NO: 160 - DNA, 309 bp, synthetic construct
  misc_difference 166: note = misc_feature - n is a, c, g, or t
  source 1..309: mol_type = other DNA
cccagtctccatcctccctgtctgcatctgtgggagacagagtcaccatcacttgccggccaagtcagagcattagcaccaatttaaattggtatcagcagaagcctgggaaagcccctaagctcctgatctatggtgcatccattttacagagtggtgacccacntagattcactggcagtagatctgggtcagatttcactctcaccatcagtggtttgcaacctgaagattttgcaacttactactgtcaacagagtgacaatactccgcggaccttcggccaagggaccaaggtggaaaccaaac

SEQ ID NO: 161 - protein, 8 aa, synthetic construct
GGSISSYY

SEQ ID NO: 162 - protein, 7 aa, synthetic construct
IFTSGST

SEQ ID NO: 163 - protein, 19 aa, synthetic construct
VRDRRGLLYSNIWYWSFDL

SEQ ID NO: 164 - protein, 6 aa, synthetic construct
QSITNY

SEQ ID NO: 165 - skipped

SEQ ID NO: 166 - protein, 9 aa, synthetic construct
QQSYSTPWT

SEQ ID NO: 167 - protein, 125 aa, synthetic construct
  VARIANT 5..7: note = misc_feature - Xaa can be any naturally occurring amino acid
  VARIANT 11: note = misc_feature - Xaa can be any naturally occurring amino acid
QVQLXXXGPGXVKPSETLSLTCSVSGGSISSYYWGWIRQPPGKGLEWIGRIFTSGSTNYNPSLESRVTMSVDMSKNQFSLSLSSVTAADTAVYYCVRDRRGLLYSNIWYWSFDLWGRGTLVTVSS

SEQ ID NO: 168 - protein, 102 aa, synthetic construct
QSPSSLSASAGDRVTITCRASQSITNYLNWYQQKPMRAPKLLIYAASTLQSGVPSRFSGSGSGTDFTLTISSLQPEDFATYYCQQSYSTPWTFGQGTKVEIR

SEQ ID NO: 169 - protein, 25 aa, synthetic construct
  VARIANT 5..7: note = misc_feature - Xaa can be any naturally occurring amino acid
  VARIANT 11: note = misc_feature - Xaa can be any naturally occurring amino acid
QVQLXXXGPGXVKPSETLSLTCSVS

SEQ ID NO: 170 - protein, 17 aa, synthetic construct
WGWIRQPPGKGLEWIGR

SEQ ID NO: 171 - protein, 38 aa, synthetic construct
NYNPSLESRVTMSVDMSKNQFSLSLSSVTAADTAVYYC

SEQ ID NO: 172 - protein, 11 aa, synthetic construct
WGRGTLVTVSS

SEQ ID NO: 173 - protein, 21 aa, synthetic construct
QSPSSLSASAGDRVTITCRAS

SEQ ID NO: 174 - protein, 17 aa, synthetic construct
LNWYQQKPMRAPKLLIY

SEQ ID NO: 175 - protein, 36 aa, synthetic construct
TLQSGVPSRFSGSGSGTDFTLTISSLQPEDFATYYC

SEQ ID NO: 176 - protein, 10 aa, synthetic construct
FGQGTKVEIR

SEQ ID NO: 177 - protein, 452 aa, synthetic construct
  VARIANT 5..7: note = misc_feature - Xaa can be any naturally occurring amino acid
  VARIANT 11: note = misc_feature - Xaa can be any naturally occurring amino acid
  VARIANT 149: note = misc_feature - Xaa can be any naturally occurring amino acid
  VARIANT 219: note = misc_feature - Xaa can be any naturally occurring amino acid
  VARIANT 363: note = misc_feature - Xaa can be any naturally occurring amino acid
QVQLXXXGPGXVKPSETLSLTCSVSGGSISSYYWGWIRQPPGKGLEWIGRIFTSGSTNYNPSLESRVTMSVDMSKNQFSLSLSSVTAADTAVYYCVRDRRGLLYSNIWYWSFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 206 aa, synthetic construct
QSPSSLSASAGDRVTITCRASQSITNYLNWYQQKPMRAPKLLIYAASTLQSGVPSRFSGSGSGTDFTLTISSLQPEDFATYYCQQSYSTPWTFGQGTKVEIRAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 179 - DNA, 376 bp, synthetic construct
  misc_difference 15..18: note = misc_feature - n is a, c, g, or t
  misc_difference 20: note = misc_feature - n is a, c, g, or t
  misc_difference 31: note = misc_feature - n is a, c, g, or t
  source 1..376: mol_type = other DNA
caggtgcagctgcannnntngggcccaggantggtgaagccttcggagaccctgtccctcacctgcagtgtctctggtggctccatcagtagttactactggggctggatccggcagccccccgggaagggactggagtggattgggcgtatttttaccagtgggagcaccaactataacccctccctcgagagtcgcgtcaccatgtcagtagacatgtccaagaaccagttctccctgagcctgagctctgtgaccgccgcggacacggccgtatattactgtgtgagagatcggaggggacttttgtatagcaacatctggtactggtccttcgatctctggggccgtggcaccctggtcactgtctcctcag

SEQ ID NO: 180 - DNA, 308 bp, synthetic construct
  source 1..308: mol_type = other DNA
ccagtctccatcctccctgtctgcatctgcaggagacagagtcaccatcacttgccgggcaagtcagagcattactaactatttaaattggtatcaacagaaaccaatgagagcccctaagctcctgatctatgctgcatccactttgcaaagtggggtcccatcaaggttcagtggcagtggctctgggacagatttcactctcaccatcagcagtctgcaacctgaagattttgcaacttactactgtcaacagagttacagtaccccgtggacgttcggccaagggaccaaggtggagatcagac

SEQ ID NO: 181 - protein, 8 aa, synthetic construct
GFPFEKFA

SEQ ID NO: 182 - protein, 8 aa, synthetic construct
ISGTGAVT

SEQ ID NO: 183 - protein, 15 aa, synthetic construct
AKDRTPVTNYYGMDV

SEQ ID NO: 184 - protein, 6 aa, synthetic construct
QTIDNY

SEQ ID NO: 185 - skipped

SEQ ID NO: 186 - protein, 9 aa, synthetic construct
QQTYSTPYT

SEQ ID NO: 187 - protein, 119 aa, synthetic construct
LVESGGNLGQPGGSLRLSCAASGFPFEKFAINWVRQAPGKGLEWVSAISGTGAVTYYADSVEGRFSISRDNSKNTVFLEMNSLRVEDTAVYFCAKDRTPVTNYYGMDVWGQGTTVTVSS

SEQ ID NO: 188 - protein, 102 aa, synthetic construct
QSPASLSASVGDRVTITCRGSQTIDNYLNWYQHKPGKAPKLLIYGAFSLQGGVPSRFSGSGYGTDFTLTISSLQPEDFATYYCQQTYSTPYTFGQGTKLETN

SEQ ID NO: 189 - protein, 22 aa, synthetic construct
LVESGGNLGQPGGSLRLSCAAS

SEQ ID NO: 190 - protein, 17 aa, synthetic construct
INWVRQAPGKGLEWVSA

SEQ ID NO: 191 - protein, 38 aa, synthetic construct
YYADSVEGRFSISRDNSKNTVFLEMNSLRVEDTAVYFC

SEQ ID NO: 192 - protein, 11 aa, synthetic construct
WGQGTTVTVSS

SEQ ID NO: 193 - protein, 21 aa, synthetic construct
QSPASLSASVGDRVTITCRGS

SEQ ID NO: 194 - protein, 17 aa, synthetic construct
LNWYQHKPGKAPKLLIY

SEQ ID NO: 195 - protein, 36 aa, synthetic construct
SLQGGVPSRFSGSGYGTDFTLTISSLQPEDFATYYC

SEQ ID NO: 196 - protein, 10 aa, synthetic construct
FGQGTKLETN

SEQ ID NO: 197 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
LVESGGNLGQPGGSLRLSCAASGFPFEKFAINWVRQAPGKGLEWVSAISGTGAVTYYADSVEGRFSISRDNSKNTVFLEMNSLRVEDTAVYFCAKDRTPVTNYYGMDVWGQGTTVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 206 aa, synthetic construct
QSPASLSASVGDRVTITCRGSQTIDNYLNWYQHKPGKAPKLLIYGAFSLQGGVPSRFSGSGYGTDFTLTISSLQPEDFATYYCQQTYSTPYTFGQGTKLETNAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 199 - DNA, 359 bp, synthetic construct
  source 1..359: mol_type = other DNA
agctggtggagtctgggggaaacttgggacagccgggggggtccctgagactgtcctgtgcagcctctggattcccctttgagaagtttgccatcaactgggtccgccaggctccagggaaggggctggagtgggtctcggctattagtggtactggtgctgtcacatactacgcagactccgtggagggccggttctccatctccagagacaattccaagaacacggtgtttctggagatgaacagcctgagagtcgaggacacggccgtgtatttctgtgcgaaagatcggactccggtgacaaactactacggcatggacgtctggggccaagggaccacggtcaccgtctcctca

SEQ ID NO: 200 - DNA, 309 bp, synthetic construct
  source 1..309: mol_type = other DNA
cccagtcgccagcgtccctgtccgcatctgtaggagacagagtcaccatcacttgccggggaagtcagaccattgacaattatttaaattggtatcagcacaaacctgggaaagcccctaaactcctgatctatggggctttcagtttgcagggtggggtcccatcaaggttcagtggcagtggatatgggacagatttcactctcaccatcagcagtctgcaacctgaagactttgcaacttactactgtcagcagacttacagtaccccgtacacttttggccaggggaccaagctggagaccaatc

SEQ ID NO: 201 - protein, 8 aa, synthetic construct
GFTFNRFA

SEQ ID NO: 202 - protein, 8 aa, synthetic construct
ISGTGAVT

SEQ ID NO: 203 - protein, 15 aa, synthetic construct
AKDRTPVTNYYGMDV

SEQ ID NO: 204 - protein, 6 aa, synthetic construct
QTIDNY

SEQ ID NO: 205 - skipped

SEQ ID NO: 206 - protein, 9 aa, synthetic construct
QQTSSTPYT

SEQ ID NO: 207 - protein, 121 aa, synthetic construct
VQLVESGGTLGQPGGSLRLSCTASGFTFNRFAINWVRQAPGKGLEWVAAISGTGAVTYYADSVEGRFSISRENSNNTVFLEMNNLRGEDTAVYFCAKDRTPVTNYYGMDVWGQGTTVTVSS

SEQ ID NO: 208 - protein, 103 aa, synthetic construct
TQSPSSLSASVGDRVIITCRGSQTIDNYLNWYQQKPGKAPRLLISAASSLQGGVPSRFSGSGYGTDFTLTISSLQPEDFATYYCQQTSSTPYTFGQGTKVEIK

SEQ ID NO: 209 - protein, 24 aa, synthetic construct
VQLVESGGTLGQPGGSLRLSCTAS

SEQ ID NO: 210 - protein, 17 aa, synthetic construct
INWVRQAPGKGLEWVAA

SEQ ID NO: 211 - protein, 38 aa, synthetic construct
YYADSVEGRFSISRENSNNTVFLEMNNLRGEDTAVYFC

SEQ ID NO: 212 - protein, 11 aa, synthetic construct
WGQGTTVTVSS

SEQ ID NO: 213 - protein, 22 aa, synthetic construct
TQSPSSLSASVGDRVIITCRGS

SEQ ID NO: 214 - protein, 17 aa, synthetic construct
LNWYQQKPGKAPRLLIS

SEQ ID NO: 215 - protein, 36 aa, synthetic construct; identical to SEQ ID NO: 195

SEQ ID NO: 216 - protein, 10 aa, synthetic construct
FGQGTKVEIK

SEQ ID NO: 217 - protein, 448 aa, synthetic construct
  VARIANT 145: note = misc_feature - Xaa can be any naturally occurring amino acid
  VARIANT 215: note = misc_feature - Xaa can be any naturally occurring amino acid
  VARIANT 359: note = misc_feature - Xaa can be any naturally occurring amino acid
VQLVESGGTLGQPGGSLRLSCTASGFTFNRFAINWVRQAPGKGLEWVAAISGTGAVTYYADSVEGRFSISRENSNNTVFLEMNNLRGEDTAVYFCAKDRTPVTNYYGMDVWGQGTTVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 207 aa, synthetic construct
TQSPSSLSASVGDRVIITCRGSQTIDNYLNWYQQKPGKAPRLLISAASSLQGGVPSRFSGSGYGTDFTLTISSLQPEDFATYYCQQTSSTPYTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 219 - DNA, 363 bp, synthetic construct
  source 1..363: mol_type = other DNA
gtgcagctggtggagtctgggggaaccctgggacagccgggggggtccctgagactctcctgtacagcctctggattcacctttaatagatttgccatcaactgggtccgccaggctccagggaaggggctggaatgggtcgccgctattagtggcactggtgctgtcacatactacgcagactccgtggagggtcggttctccatctccagagagaattccaacaacacggtttttctggaaatgaacaacctgagaggcgaagacacggccgtatatttctgtgcgaaagatcggactccggtgacaaactattacggcatggacgtctggggccaagggaccacggtcaccgtctcctca

SEQ ID NO: 220 - DNA, 312 bp, synthetic construct
  source 1..312: mol_type = other DNA
agacccagtctccatcgtccctgtctgcatctgtgggagacagagtcatcatcacttgccggggaagtcagaccattgacaattatttaaattggtatcagcagaaacctgggaaagcccccaggctccttatctctgcggcatccagcttgcaagggggggtcccttcaaggttcagtggcagtggatatgggacggatttcaccctcaccatcagcagtctgcaacctgaagattttgcaacttactactgtcagcagacttccagtaccccgtacacttttggccaggggaccaaggtggagatcaaac

SEQ ID NO: 221 - protein, 8 aa, synthetic construct
GFTFSIFG

SEQ ID NO: 222 - protein, 8 aa, synthetic construct
ISGTGEIT

SEQ ID NO: 223 - protein, 15 aa, synthetic construct
AKERTKYQLAYPFDY

SEQ ID NO: 224 - protein, 6 aa, synthetic construct
QDITTY

SEQ ID NO: 225 - skipped

SEQ ID NO: 226 - protein, 9 aa, synthetic construct
QQYENFPRT

SEQ ID NO: 227 - protein, 121 aa, synthetic construct
VQLVESGGGLVQPGGSLRLSCTASGFTFSIFGMSWVRQAPGKGLEWVSSISGTGEITKYTDSVKGRFTISRDNSKTTVYLQMKSLRAEDTAFYFCAKERTKYQLAYPFDYWGQGTLVTVSS

SEQ ID NO: 228 - protein, 103 aa, synthetic construct
TQSPSSLSASVGDRVTITCQASQDITTYLSWYQQIPGKAPKLLISDASFLQAGVPSRFSGSGSGTDFTFTITNLQPEDVATYYCQQYENFPRTFGGGTKVEIR

SEQ ID NO: 229 - protein, 24 aa, synthetic construct
VQLVESGGGLVQPGGSLRLSCTAS

SEQ ID NO: 230 - protein, 17 aa, synthetic construct
MSWVRQAPGKGLEWVSS

SEQ ID NO: 231 - protein, 38 aa, synthetic construct
KYTDSVKGRFTISRDNSKTTVYLQMKSLRAEDTAFYFC

SEQ ID NO: 232 - protein, 11 aa, synthetic construct
WGQGTLVTVSS

SEQ ID NO: 233 - protein, 22 aa, synthetic construct
TQSPSSLSASVGDRVTITCQAS

SEQ ID NO: 234 - protein, 17 aa, synthetic construct
LSWYQQIPGKAPKLLIS

SEQ ID NO: 235 - protein, 36 aa, synthetic construct
FLQAGVPSRFSGSGSGTDFTFTITNLQPEDVATYYC

SEQ ID NO: 236 - protein, 10 aa, synthetic construct
FGGGTKVEIR

SEQ ID NO: 237 - protein, 448 aa, synthetic construct
  VARIANT 145: note = misc_feature - Xaa can be any naturally occurring amino acid
  VARIANT 215: note = misc_feature - Xaa can be any naturally occurring amino acid
  VARIANT 359: note = misc_feature - Xaa can be any naturally occurring amino acid
VQLVESGGGLVQPGGSLRLSCTASGFTFSIFGMSWVRQAPGKGLEWVSSISGTGEITKYTDSVKGRFTISRDNSKTTVYLQMKSLRAEDTAFYFCAKERTKYQLAYPFDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 207 aa, synthetic construct
TQSPSSLSASVGDRVTITCQASQDITTYLSWYQQIPGKAPKLLISDASFLQAGVPSRFSGSGSGTDFTFTITNLQPEDVATYYCQQYENFPRTFGGGTKVEIRAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 239 - DNA, 364 bp, synthetic construct
  source 1..364: mol_type = other DNA
gtgcagctggtggagtctgggggaggcttggtacagcctggggggtccctgagactctcgtgtactgcctctggcttcacttttagcatctttggcatgagttgggtccgccaggctccagggaaggggctggagtgggtctcaagtatcagtggtactggtgaaattacgaaatacacagactccgtgaagggccggttcaccatctccagagacaattccaagactaccgtgtatctgcagatgaagagcctgagagccgaggacacggccttttatttctgtgcaaaagaaagaacgaagtatcaattggcatacccctttgactactggggccagggaaccctggtcaccgtctcctcgg

SEQ ID NO: 240 - DNA, 310 bp, synthetic construct
  source 1..310: mol_type = other DNA
acccagtctccatcctccctgtctgcttctgttggagacagagtcaccatcacttgccaggcgagtcaagacattaccacttatttgagttggtatcagcagataccagggaaagcccctaaactcctgatctccgatgcatcctttttgcaagcaggggtcccttcaagattcagtggaagtgggtctgggacagattttactttcaccatcaccaacctgcagcctgaggatgttgcaacatactactgtcaacagtatgagaatttcccgcgaactttcggcggagggaccaaggtggagatcagac

SEQ ID NO: 241 - protein, 8 aa, synthetic construct
GDSISSYY

SEQ ID NO: 242 - protein, 7 aa, synthetic construct
IFTSGST

SEQ ID NO: 243 - protein, 20 aa, synthetic construct
AKHTRDFWSDDSRVHWYFNL

SEQ ID NO: 244 - protein, 7 aa, synthetic construct
QSVSNNF

SEQ ID NO: 245 - skipped

SEQ ID NO: 246 - protein, 9 aa, synthetic construct
QQYDSSPYT

SEQ ID NO: 247 - protein, 121 aa, synthetic construct
ESGPGLVKPSETLSLTCTVSGDSISSYYWSWIRQPAGKGLEWIGRIFTSGSTNINPSLKSRVTMSVDTSKNQFSLQLGSVTAADTAIYYCAKHTRDFWSDDSRVHWYFNLWGRGTLVTVSS

SEQ ID NO: 248 - protein, 103 aa, synthetic construct
QSPGTLSLSPGERATLSCRASQSVSNNFLAWYQQQPGQAPSLLIYGASTRATGIPDRFSGSGSGTDFTLTISRLEPEDFAVYFCQQYDSSPYTFGQGTKLEIK

SEQ ID NO: 249 - protein, 20 aa, synthetic construct
ESGPGLVKPSETLSLTCTVS

SEQ ID NO: 250 - protein, 17 aa, synthetic construct
WSWIRQPAGKGLEWIGR

SEQ ID NO: 251 - protein, 38 aa, synthetic construct
NINPSLKSRVTMSVDTSKNQFSLQLGSVTAADTAIYYC

SEQ ID NO: 252 - protein, 11 aa, synthetic construct
WGRGTLVTVSS

SEQ ID NO: 253 - protein, 21 aa, synthetic construct
QSPGTLSLSPGERATLSCRAS

SEQ ID NO: 254 - protein, 17 aa, synthetic construct
LAWYQQQPGQAPSLLIY

SEQ ID NO: 255 - protein, 36 aa, synthetic construct
TRATGIPDRFSGSGSGTDFTLTISRLEPEDFAVYFC

SEQ ID NO: 256 - protein, 10 aa, synthetic construct
FGQGTKLEIK

SEQ ID NO: 257 - protein, 448 aa, synthetic construct
  VARIANT 145: note = misc_feature - Xaa can be any naturally occurring amino acid
  VARIANT 215: note = misc_feature - Xaa can be any naturally occurring amino acid
  VARIANT 359: note = misc_feature - Xaa can be any naturally occurring amino acid
ESGPGLVKPSETLSLTCTVSGDSISSYYWSWIRQPAGKGLEWIGRIFTSGSTNINPSLKSRVTMSVDTSKNQFSLQLGSVTAADTAIYYCAKHTRDFWSDDSRVHWYFNLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 207 aa, synthetic construct
QSPGTLSLSPGERATLSCRASQSVSNNFLAWYQQQPGQAPSLLIYGASTRATGIPDRFSGSGSGTDFTLTISRLEPEDFAVYFCQQYDSSPYTFGQGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 259 - DNA, 365 bp, synthetic construct
  source 1..365: mol_type = other DNA
ggagtcgggcccaggactggtgaagccttcggagaccctgtccctcacctgcactgtctctggtgactccatcagtagttattattggagctggatccggcagcccgccgggaagggactggagtggatcgggcgtatcttcaccagtgggagcaccaacatcaacccctccctcaagagtcgagtcaccatgtcagtagacacgtccaaaaatcagttctccctgcagctgggttcggtgaccgccgcggacacggccatctattattgtgcgaaacatacccgcgatttttggagtgatgactctcgggtccactggtacttcaatctctggggccgtggcaccctggtcactgtctcctcag

SEQ ID NO: 260 - DNA, 310 bp, synthetic construct
  source 1..310: mol_type = other DNA
cagtctccaggcaccctgtctttgtctccaggggaaagagccaccctctcctgcagggccagtcagagtgttagcaacaacttcttagcctggtaccagcagcaacctggccaggctcccagtctcctcatctatggtgcttccactagggccactggcatcccagacaggttcagtggcagtgggtctgggacagacttcactctcaccatcagcagactggagcctgaagattttgcagtgtatttctgtcagcaatatgatagctcaccgtacacttttggccaggggaccaagctggagatcaaac

SEQ ID NO: 261 - protein, 8 aa, synthetic construct
GFNFDDYT

SEQ ID NO: 262 - protein, 8 aa, synthetic construct
IKWNSNNI

SEQ ID NO: 263 - protein, 11 aa, synthetic construct
VKDNGFRGFHI

SEQ ID NO: 264 - protein, 11 aa, synthetic construct
QSLLHRNGYNY

SEQ ID NO: 265 - skipped

SEQ ID NO: 266 - protein, 8 aa, synthetic construct
MQALESWT

SEQ ID NO: 267 - protein, 117 aa, synthetic construct
  VARIANT 2..3: note = misc_feature - Xaa can be any naturally occurring amino acid
VXXVESGGGFVQPGGSLRLSCVASGFNFDDYTMHWVRQVAGTGLEWVSSIKWNSNNIDYADSVKGRFTISRDNAKNSLFLQMDSLRVEDTAFYYCVKDNGFRGFHIWGQGTMVTVSS

SEQ ID NO: 268 - protein, 107 aa, synthetic construct
TQSPLSLSVTPGESASISCRSSQSLLHRNGYNYLDWYLQKPGQSPQLLIHLGSKRASGVPDRFSGSGSGTDFTLKISRVEAEDVGVYYCMQALESWTFGQGTKVEIK

SEQ ID NO: 269 - protein, 24 aa, synthetic construct
  VARIANT 2..3: note = misc_feature - Xaa can be any naturally occurring amino acid
VXXVESGGGFVQPGGSLRLSCVAS

SEQ ID NO: 270 - protein, 17 aa, synthetic construct
MHWVRQVAGTGLEWVSS

SEQ ID NO: 271 - protein, 38 aa, synthetic construct
DYADSVKGRFTISRDNAKNSLFLQMDSLRVEDTAFYYC

SEQ ID NO: 272 - protein, 11 aa, synthetic construct
WGQGTMVTVSS

SEQ ID NO: 273 - protein, 22 aa, synthetic construct
TQSPLSLSVTPGESASISCRSS

SEQ ID NO: 274 - protein, 17 aa, synthetic construct
LDWYLQKPGQSPQLLIH

SEQ ID NO: 275 - protein, 36 aa, synthetic construct
KRASGVPDRFSGSGSGTDFTLKISRVEAEDVGVYYC

SEQ ID NO: 276 - protein, 10 aa, synthetic construct
FGQGTKVEIK

SEQ ID NO: 277 - protein, 444 aa, synthetic construct
  VARIANT 2..3: note = misc_feature - Xaa can be any naturally occurring amino acid
  VARIANT 141: note = misc_feature - Xaa can be any naturally occurring amino acid
  VARIANT 211: note = misc_feature - Xaa can be any naturally occurring amino acid
  VARIANT 355: note = misc_feature - Xaa can be any naturally occurring amino acid
VXXVESGGGFVQPGGSLRLSCVASGFNFDDYTMHWVRQVAGTGLEWVSSIKWNSNNIDYADSVKGRFTISRDNAKNSLFLQMDSLRVEDTAFYYCVKDNGFRGFHIWGQGTMVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 211 aa, synthetic construct
TQSPLSLSVTPGESASISCRSSQSLLHRNGYNYLDWYLQKPGQSPQLLIHLGSKRASGVPDRFSGSGSGTDFTLKISRVEAEDVGVYYCMQALESWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 279 - DNA, 354 bp, synthetic construct
  misc_difference 6: note = misc_feature - n is a, c, g, or t
  misc_difference 10: note = misc_feature - n is a, c, g, or t
  source 1..354: mol_type = other DNA
aggtgncagnggtggagtctgggggaggcttcgtacagcctggcgggtccctgagactctcctgtgtagcctctggattcaactttgatgattataccatgcactgggtccgacaagttgcagggacgggcctggagtgggtctcaagtattaaatggaactctaataacatagactatgcggactctgtgaagggccggttcaccatctccagagacaacgccaagaattccctgtttctgcagatggatagtctcagagttgaagacacggccttctattactgtgtaaaagataatggcttccgtggttttcatatatggggccaagggacaatggtcaccgtctcttcag

SEQ ID NO: 280 - DNA, 323 bp, synthetic construct
  source 1..323: mol_type = other DNA
gactcagtctccactctccctgtccgtcacccctggagagtcggcctccatctcctgcaggtctagtcagagcctcctgcatcgtaatggatacaactatttggattggtacctgcagaagccagggcagtctccacaactcctgatccatttgggttctaagcgggcctccggggtccctgacaggttcagtggcagtggatcaggcacagattttacactgaaaatcagcagagtggaggctgaggatgttggggtttattactgcatgcaagctctagaaagctggacgttcggccaagggaccaaggtggaaatcaaac

SEQ ID NO: 281 - protein, 8 aa, synthetic construct
GYTFTTYA

SEQ ID NO: 282 - protein, 8 aa, synthetic construct
INAGNGNP

SEQ ID NO: 283 - protein, 14 aa, synthetic construct
ARDAAGTRGNWLDP

SEQ ID NO: 284 - protein, 12 aa, synthetic construct
QSVLYSSNNKNY

SEQ ID NO: 285 - skipped

SEQ ID NO: 286 - protein, 9 aa, synthetic construct
QQYYSTPLT

SEQ ID NO: 287 - protein, 116 aa, synthetic construct
QSGAEVKKPGASVKISCKASGYTFTTYAMHWVRQAPGQRLEWMGGINAGNGNPKYSEKFQDRVTITRDTSATTAYMELSSLRSEDTAVYYCARDAAGTRGNWLDPWGQGTLVTVSS

SEQ ID NO: 288 - protein, 108 aa, synthetic construct
QSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 289 - protein, 20 aa, synthetic construct
QSGAEVKKPGASVKISCKAS

SEQ ID NO: 290 - protein, 17 aa, synthetic construct
MHWVRQAPGQRLEWMGG

SEQ ID NO: 291 - protein, 38 aa, synthetic construct
KYSEKFQDRVTITRDTSATTAYMELSSLRSEDTAVYYC

SEQ ID NO: 292 - protein, 11 aa, synthetic construct
WGQGTLVTVSS

SEQ ID NO: 293 - protein, 21 aa, synthetic construct
QSPDSLAVSLGERATINCKSS

SEQ ID NO: 294 - protein, 17 aa, synthetic construct
LAWYQQKPGQPPKLLIY

SEQ ID NO: 295 - protein, 36 aa, synthetic construct
TRESGVPDRFSGSGSGTDFTLTISSLQAEDVAVYYC

SEQ ID NO: 296 - protein, 10 aa, synthetic construct
FGGGTKVEIK

SEQ ID NO: 297 - protein, 443 aa, synthetic construct
  VARIANT 140: note = misc_feature - Xaa can be any naturally occurring amino acid
  VARIANT 210: note = misc_feature - Xaa can be any naturally occurring amino acid
  VARIANT 354: note = misc_feature - Xaa can be any naturally occurring amino acid
QSGAEVKKPGASVKISCKASGYTFTTYAMHWVRQAPGQRLEWMGGINAGNGNPKYSEKFQDRVTITRDTSATTAYMELSSLRSEDTAVYYCARDAAGTRGNWLDPWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 212 aa, synthetic construct
QSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 299 - DNA, 350 bp, synthetic construct
  source 1..350: mol_type = other DNA
gcagtccggggctgaggtgaagaagcctggggcctcagtgaagatttcctgcaaggcttctggatacaccttcactacttatgctatgcattgggtgcgccaggcccccggacaaaggcttgagtggatgggagggatcaacgctggcaatggtaatccaaaatattcagagaagttccaggacagagtcaccattaccagggacacatccgcgaccacagcctacatggagctgagcagcctgagatctgaggacacggctgtgtattactgtgcgagagatgcggccggcactcgaggaaactggctcgacccctggggccagggtaccctggtcaccgtctcctcag

SEQ ID NO: 300 - DNA, 326 bp, synthetic construct
  source 1..326: mol_type = other DNA
ccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctactcgggaatccggggtccctgaccgattcagtggcagcgggtctgggacagatttcactctcaccatcagcagcctgcaggctgaagatgtggcagtttattactgtcagcaatattatagtactcctctcactttcggcggagggaccaaggtggagatcaaac

SEQ ID NO: 301 - protein, 8 aa, synthetic construct
GFTFTTYA

SEQ ID NO: 302 - protein, 8 aa, synthetic construct
ISGSGGST

SEQ ID NO: 303 - protein, 14 aa, synthetic construct
AKDWSSGYLGYFDY

SEQ ID NO: 304 - protein, 12 aa, synthetic construct
QSVLYSSNNKNY

SEQ ID NO: 305 - skipped

SEQ ID NO: 306 - protein, 9 aa, synthetic construct
QQYYSTPLT

SEQ ID NO: 307 - protein, 117 aa, synthetic construct
VESGGGLVQPGGSLRLSCAASGFTFTTYAMTWVRQAPGKGLEWVSAISGSGGSTYYADSVKGRFTISRDNSKNTLYLQMNSLRAEDTAVYYCAKDWSSGYLGYFDYWGQGTLVTVSS

SEQ ID NO: 308 - protein, 108 aa, synthetic construct
  VARIANT 99: note = misc_feature - Xaa can be any naturally occurring amino acid
QSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTXGGGTKVEIK

SEQ ID NO: 309 - protein, 21 aa, synthetic construct
VESGGGLVQPGGSLRLSCAAS

SEQ ID NO: 310 - protein, 17 aa, synthetic construct
MTWVRQAPGKGLEWVSA

SEQ ID NO: 311 - protein, 38 aa, synthetic construct
YYADSVKGRFTISRDNSKNTLYLQMNSLRAEDTAVYYC

SEQ ID NO: 312 - protein, 11 aa, synthetic construct
WGQGTLVTVSS

SEQ ID NO: 313 - protein, 21 aa, synthetic construct
QSPDSLAVSLGERATINCKSS

SEQ ID NO: 314 - protein, 17 aa, synthetic construct
LAWYQQKPGQPPKLLIY

SEQ ID NO: 315 - protein, 36 aa, synthetic construct; identical to SEQ ID NO: 295

SEQ ID NO: 316 - protein, 10 aa, synthetic construct
  VARIANT 1: note = misc_feature - Xaa can be any naturally occurring amino acid
XGGGTKVEIK

SEQ ID NO: 317 - protein, 444 aa, synthetic construct
  VARIANT 141: note = misc_feature - Xaa can be any naturally occurring amino acid
  VARIANT 211: note = misc_feature - Xaa can be any naturally occurring amino acid
  VARIANT 355: note = misc_feature - Xaa can be any naturally occurring amino acid
VESGGGLVQPGGSLRLSCAASGFTFTTYAMTWVRQAPGKGLEWVSAISGSGGSTYYADSVKGRFTISRDNSKNTLYLQMNSLRAEDTAVYYCAKDWSSGYLGYFDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 212 aa, synthetic construct
  VARIANT 99: note = misc_feature - Xaa can be any naturally occurring amino acid
QSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTXGGGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 319 - DNA, 353 bp, synthetic construct
  misc_difference 4: note = misc_feature - n is a, c, g, or t
  source 1..353: mol_type = other DNA
ggtngagtctgggggaggcttggtacagcctggggggtccctgagactctcctgtgcagcctctggattcacctttaccacctatgccatgacctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattggagtagtggttatttgggctactttgactactggggccagggaaccctggtcaccgtctcctcag

SEQ ID NO: 320 - DNA, 326 bp, synthetic construct
  misc_difference 297: note = misc_feature - n is a, c, g, or t
  source 1..326: mol_type = other DNA
ccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctctcacttncggcggagggaccaaggtggagatcaaac

SEQ ID NO: 321 - protein, 8 aa, synthetic construct
GFVFDDYS

SEQ ID NO: 322 - protein, 8 aa, synthetic construct
ISWDGGRT

SEQ ID NO: 323 - protein, 11 aa, synthetic construct
VKDTGLRSFDS

SEQ ID NO: 324 - protein, 11 aa, synthetic construct
QSLMHTNGYIY

SEQ ID NO: 325 - skipped

SEQ ID NO: 326 - protein, 8 aa, synthetic construct
MQALQSWT

SEQ ID NO: 327 - protein, 113 aa, synthetic construct
ESGGEVVQPGRSLRLSCAASGFVFDDYSMHWVRQVPGKGLEWVALISWDGGRTSYGHSVKGRFTISRDNTKDSLFLQMNSLRPEDTALYYCVKDTGLRSFDSWGQGILVTVSS

SEQ ID NO: 328 - protein, 111 aa, synthetic construct
  VARIANT 2..4: note = misc_feature - Xaa can be any naturally occurring amino acid
MXXXTQSPLSLPVTPGEPASISCRSSQSLMHTNGYIYLDWYLQKPGQSPQLLIYLGSKRAPGVPDRFSGSGSGTDFTLKISRVEAEDVGVYYCMQALQSWTFGQGTKVEIK

SEQ ID NO: 329 - protein, 20 aa, synthetic construct
ESGGEVVQPGRSLRLSCAAS

SEQ ID NO: 330 - protein, 17 aa, synthetic construct
MHWVRQVPGKGLEWVAL

SEQ ID NO: 331 - protein, 38 aa, synthetic construct
SYGHSVKGRFTISRDNTKDSLFLQMNSLRPEDTALYYC

SEQ ID NO: 332 - protein, 11 aa, synthetic construct
WGQGILVTVSS

SEQ ID NO: 333 - protein, 26 aa, synthetic construct
  VARIANT 2..4: note = misc_feature - Xaa can be any naturally occurring amino acid
MXXXTQSPLSLPVTPGEPASISCRSS

SEQ ID NO: 334 - protein, 17 aa, synthetic construct
LDWYLQKPGQSPQLLIY

SEQ ID NO: 335 - protein, 36 aa, synthetic construct
KRAPGVPDRFSGSGSGTDFTLKISRVEAEDVGVYYC

SEQ ID NO: 336 - protein, 10 aa, synthetic construct
FGQGTKVEIK

SEQ ID NO: 337 - protein, 440 aa, synthetic construct
  VARIANT 137: note = misc_feature - Xaa can be any naturally occurring amino acid
  VARIANT 207: note = misc_feature - Xaa can be any naturally occurring amino acid
  VARIANT 351: note = misc_feature - Xaa can be any naturally occurring amino acid
ESGGEVVQPGRSLRLSCAASGFVFDDYSMHWVRQVPGKGLEWVALISWDGGRTSYGHSVKGRFTISRDNTKDSLFLQMNSLRPEDTALYYCVKDTGLRSFDSWGQGILVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 215 aa, synthetic construct
  VARIANT 2..4: note = misc_feature - Xaa can be any naturally occurring amino acid
MXXXTQSPLSLPVTPGEPASISCRSSQSLMHTNGYIYLDWYLQKPGQSPQLLIYLGSKRAPGVPDRFSGSGSGTDFTLKISRVEAEDVGVYYCMQALQSWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 339 - DNA, 342 bp, synthetic construct
  source 1..342: mol_type = other DNA
tggagtctggcggagaggtcgtacagccggggaggtccctgaggctgtcctgtgcagcctctggattcgtctttgatgattattccatgcactgggtccgtcaagtcccggggaagggccttgagtgggtcgctctaattagttgggatggtggtcgcacatcctatggacactctgtgaagggccgattcaccatctccagagacaacaccaaggactccctgtttctgcaaatgaacagtctgagacccgaggacaccgccctttattactgtgtaaaagatactggacttcgaagctttgactcttggggccagggaattctggtcaccgtctcgtcag

SEQ ID NO: 340 - DNA, 334 bp, synthetic construct
  misc_difference 6: note = misc_feature - n is a, c, g, or t
  misc_difference 8..10: note = misc_feature - n is a, c, g, or t
  source 1..334: mol_type = other DNA
atgttngnnnagactcagtctccactctccctgcccgtcactcctggagagccggcctccatctcctgcaggtctagtcagagcctcatgcatactaatggatacatctatttggattggtacctgcagaagcctgggcagtctccacagctcctgatctatctgggttctaagcgggcccccggggtccccgacaggttcagtggcagtggatcaggcacagattttacactgaaaatcagcagagtggaggctgaggatgttggggtttattactgcatgcaagctctacaaagttggacgttcggccaagggaccaaggtggaaatcaaac

SEQ ID NO: 341 - protein, 8 aa, synthetic construct
GFTFSHAS

SEQ ID NO: 342 - protein, 10 aa, synthetic construct
IRSKTDGGTT

SEQ ID NO: 343 - protein, 15 aa, synthetic construct
NTHFYCNRSMCYGDY

SEQ ID NO: 344 - protein, 12 aa, synthetic construct
QSVLYSSNNKNY

SEQ ID NO: 345 - skipped

SEQ ID NO: 346 - protein, 8 aa, synthetic construct
QQYYSGRT

SEQ ID NO: 347 - protein, 119 aa, synthetic construct
ESGGGLVKPGGSLRLSCAASGFTFSHASMSWVRQAPGKGLEWVGRIRSKTDGGTTDYAAPVKGRFTISRDDSKNTLYLQMNSLKTEDTAVYYCNTHFYCNRSMCYGDYWGQGTLVTVSS

SEQ ID NO: 348 - protein, 112 aa, synthetic construct
  VARIANT 3..4: note = misc_feature - Xaa can be any naturally occurring amino acid
DIXXTQSPDSLAVSLGERATINCKSSQSVLYSSNNKNYLAWYQQKPGQPPKLLIYWASTRESGVPDRFRGSGSGTDFSLTISSLQAEDVAVYYCQQYYSGRTFGPGTKVDFK

SEQ ID NO: 349 - protein, 20 aa, synthetic construct
ESGGGLVKPGGSLRLSCAAS

SEQ ID NO: 350 - protein, 17 aa, synthetic construct
MSWVRQAPGKGLEWVGR

SEQ ID NO: 351 - protein, 38 aa, synthetic construct
DYAAPVKGRFTISRDDSKNTLYLQMNSLKTEDTAVYYC

SEQ ID NO: 352 - protein, 11 aa, synthetic construct
WGQGTLVTVSS

SEQ ID NO: 353 - protein, 26 aa, synthetic construct
  VARIANT 3..4: note = misc_feature - Xaa can be any naturally occurring amino acid
DIXXTQSPDSLAVSLGERATINCKSS

SEQ ID NO: 354 - protein, 17 aa, synthetic construct
LAWYQQKPGQPPKLLIY

SEQ ID NO: 355 - protein, 36 aa, synthetic construct
TRESGVPDRFRGSGSGTDFSLTISSLQAEDVAVYYC

SEQ ID NO: 356 - protein, 10 aa, synthetic construct
FGPGTKVDFK

SEQ ID NO: 357 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
ESGGGLVKPGGSLRLSCAASGFTFSHASMSWVRQAPGKGLEWVGRIRSKTDGGTTDYAAPVKGRFTISRDDSKNTLYLQMNSLKTEDTAVYYCNTHFYCNRSMCYGDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 216 aa, synthetic construct
  VARIANT 3..4: note = misc_feature - Xaa can be any naturally occurring amino acid
DIXXTQSPDSLAVSLGERATINCKSSQSVLYSSNNKNYLAWYQQKPGQPPKLLIYWASTRESGVPDRFRGSGSGTDFSLTISSLQAEDVAVYYCQQYYSGRTFGPGTKVDFKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 359 - DNA, 360 bp, synthetic construct
  misc_difference 2: note = misc_feature - n is a, c, g, or t
  source 1..360: mol_type = other DNA
tngagtctgggggaggcttggtaaagcctggggggtcccttagactctcctgtgcagcctctggattcactttcagtcacgcctcgatgagctgggtccgccaggctccagggaaggggctggagtgggttggccgtattagaagcaaaactgatggcgggacaacagactacgctgcacccgtgaaaggcagattcaccatctcaagagatgattcaaaaaacacactgtatctgcaaatgaacagcctgaaaaccgaggacacagccgtgtattactgtaatacccacttctattgcaatagaagcatgtgctatggtgactactggggccagggaaccctggtcaccgtctcctcag

SEQ ID NO: 360 - DNA, 337 bp, synthetic construct
  misc_difference 8..10: note = misc_feature - n is a, c, g, or t
  source 1..337: mol_type = other DNA
gacatcgnnnagacccagtctccagactccctggctgtgtctctgggcgagagggccaccatcaactgcaagtccagccagagtgttttatacagctccaataataagaattacttagcttggtaccagcagaaaccagggcagcctcctaagttgctcatttactgggcatctacccgggaatccggggtccctgaccgattcagaggcagcgggtctgggacagatttctctctcaccatcagcagcctgcaggctgaagatgtggcagtttattactgtcagcaatattatagtggtcgcactttcggccctgggaccaaagtggatttcaaac

SEQ ID NO: 361 - protein, 8 aa, synthetic construct
GFTFEDYS

SEQ ID NO: 362 - protein, 8 aa, synthetic construct
ISWDGGRT

SEQ ID NO: 363 - protein, 11 aa, synthetic construct
VKDTGLRSFDS

SEQ ID NO: 364 - protein, 11 aa, synthetic construct
QSLVHSNGINY

SEQ ID NO: 365 - skipped

SEQ ID NO: 366 - protein, 8 aa, synthetic construct
MQALQSWT

SEQ ID NO: 367 - protein, 114 aa, synthetic construct
VESGGVVVQRGGSLRLSCAGSGFTFEDYSMHWVRQSPGKGLEWVSLISWDGGRTSYGDSVKGRFTISRDNSKKSLFLQMTNLRPEDTALYYCVKDTGLRSFDSWGQGTLVTVSS

SEQ ID NO: 368 - protein, 111 aa, synthetic construct
  VARIANT 2..4: note = misc_feature - Xaa can be any naturally occurring amino acid
MXXXTQSPLSLPVTPGEPASISCRSSQSLVHSNGINYLDWYIQKPGQSPQLLIYMGFKRAAGVPDRFSGSVSGTDFTLMISRVEAEDVGVYYCMQALQSWTFGQGTRVEIK

SEQ ID NO: 369 - protein, 21 aa, synthetic construct
VESGGVVVQRGGSLRLSCAGS

SEQ ID NO: 370 - protein, 17 aa, synthetic construct
MHWVRQSPGKGLEWVSL

SEQ ID NO: 371 - protein, 38 aa, synthetic construct
SYGDSVKGRFTISRDNSKKSLFLQMTNLRPEDTALYYC

SEQ ID NO: 372 - protein, 11 aa, synthetic construct
WGQGTLVTVSS

SEQ ID NO: 373 - protein, 26 aa, synthetic construct
  VARIANT 2..4: note = misc_feature - Xaa can be any naturally occurring amino acid
MXXXTQSPLSLPVTPGEPASISCRSS

SEQ ID NO: 374 - protein, 17 aa, synthetic construct
LDWYIQKPGQSPQLLIY

SEQ ID NO: 375 - protein, 36 aa, synthetic construct
KRAAGVPDRFSGSVSGTDFTLMISRVEAEDVGVYYC

SEQ ID NO: 376 - protein, 10 aa, synthetic construct
FGQGTRVEIK

SEQ ID NO: 377 - protein, 441 aa, synthetic construct
  VARIANT 138: note = misc_feature - Xaa can be any naturally occurring amino acid
  VARIANT 208: note = misc_feature - Xaa can be any naturally occurring amino acid
  VARIANT 352: note = misc_feature - Xaa can be any naturally occurring amino acid
VESGGVVVQRGGSLRLSCAGSGFTFEDYSMHWVRQSPGKGLEWVSLISWDGGRTSYGDSVKGRFTISRDNSKKSLFLQMTNLRPEDTALYYCVKDTGLRSFDSWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 215 aa, synthetic construct
  VARIANT 2..4: note = misc_feature - Xaa can be any naturally occurring amino acid
MXXXTQSPLSLPVTPGEPASISCRSSQSLVHSNGINYLDWYIQKPGQSPQLLIYMGFKRAAGVPDRFSGSVSGTDFTLMISRVEAEDVGVYYCMQALQSWTFGQGTRVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 379 - DNA, 343 bp, synthetic construct
  source 1..343: mol_type = other DNA
gtggagtctggcggagtcgtggtacagcggggggggtccctgagactctcctgtgcaggctctggattcacctttgaggattattccatgcactgggtccgtcaatctccggggaagggtcttgagtgggtctctcttattagttgggatggtggccggacatcctatggagactctgtgaaggggcggttcaccatctccagagacaacagcaagaagtccctgtttctgcaaatgaccaatctgagacccgaagacaccgccctctattattgtgtaaaagatactggactccgaagttttgactcttggggccagggaaccctggtcaccgtctcctcag

SEQ ID NO: 380 - DNA, 334 bp, synthetic construct
  misc_difference 6: note = misc_feature - n is a, c, g, or t
  misc_difference 8..9: note = misc_feature - n is a, c, g, or t
  misc_difference 11: note = misc_feature - n is a, c, g, or t
  source 1..334: mol_type = other DNA
atgttngnngngactcagtctcctctctccctgcccgtcacccctggagagccggcctccatctcctgtcggtctagtcagagcctcgtgcatagtaatggaatcaactatttggactggtacatccagaagccagggcagtctccacaactcctgatctatatgggttttaagcgggccgccggggtccctgacaggttcagtggcagtgtctcaggcacagattttacactgatgatcagcagagtggaggctgaggatgttggggtctattactgcatgcaagctctacaaagttggacgttcggccaagggaccagggtggaaatcaaac

SEQ ID NO: 381 - protein, 8 aa, synthetic construct
GFIFDDYS

SEQ ID NO: 382 - protein, 8 aa, synthetic construct
ISWDGGRT

SEQ ID NO: 383 - protein, 11 aa, synthetic construct
VKDTGLRSFDS

SEQ ID NO: 384 - protein, 11 aa, synthetic construct
QSLVHSNGYTY

SEQ ID NO: 385 - skipped

SEQ ID NO: 386 - protein, 8 aa, synthetic construct
MQALQSWT

SEQ ID NO: 387 - protein, 113 aa, synthetic construct
ESGGEVVQPGGSLRLSCVASGFIFDDYSMHWVRQVPGKGLEWVSLISWDGGRTSYGDSVKGRFTISRDNNKKTLFLQMNSLRPEDTALYYCVKDTGLRSFDSWGQGILVTVSS

SEQ ID NO: 388 - protein, 111 aa, synthetic construct
  VARIANT 3: note = misc_feature - Xaa can be any naturally occurring amino acid
DVXETQSPLSLPVTPGEPASISCRSSQSLVHSNGYTYLDWYLQRPGQSPQLLIYMGSKRASGVPDRFSGSFSGTDFTLKISRVEAEDVGVYYCMQALQSWTFGLGTKVEIK

SEQ ID NO: 389 - protein, 20 aa, synthetic construct
ESGGEVVQPGGSLRLSCVAS

SEQ ID NO: 390 - protein, 17 aa, synthetic construct
MHWVRQVPGKGLEWVSL

SEQ ID NO: 391 - protein, 38 aa, synthetic construct
SYGDSVKGRFTISRDNNKKTLFLQMNSLRPEDTALYYC

SEQ ID NO: 392 - protein, 11 aa, synthetic construct
WGQGILVTVSS

SEQ ID NO: 393 - protein, 26 aa, synthetic construct
  VARIANT 3: note = misc_feature - Xaa can be any naturally occurring amino acid
DVXETQSPLSLPVTPGEPASISCRSS

SEQ ID NO: 394 - protein, 17 aa, synthetic construct
LDWYLQRPGQSPQLLIY

SEQ ID NO: 395 - protein, 36 aa, synthetic construct
KRASGVPDRFSGSFSGTDFTLKISRVEAEDVGVYYC

SEQ ID NO: 396 - protein, 10 aa, synthetic construct
FGLGTKVEIK

SEQ ID NO: 397 - protein, 440 aa, synthetic construct
  VARIANT 137: note = misc_feature - Xaa can be any naturally occurring amino acid
  VARIANT 207: note = misc_feature - Xaa can be any naturally occurring amino acid
  VARIANT 351: note = misc_feature - Xaa can be any naturally occurring amino acid
ESGGEVVQPGGSLRLSCVASGFIFDDYSMHWVRQVPGKGLEWVSLISWDGGRTSYGDSVKGRFTISRDNNKKTLFLQMNSLRPEDTALYYCVKDTGLRSFDSWGQGILVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 398 - protein, 215 aa, synthetic construct
  VARIANT 3: note = misc_feature - Xaa can be any naturally occurring amino acid
DVXETQSPLSLPVTPGEPASISCRSSQSLVHSNGYTYLDWYLQRPGQSPQLLIYMGSKRASGVPDRFSGSFSGTDFTLKISRVEAEDVGVYYCMQALQSWTFGLGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 399 - DNA, 341 bp, synthetic construct
  source 1..341: mol_type = other DNA
ggagtctggcggagaggtcgtacagcctggggggtccctgagactctcttgtgtagcctctggattcatctttgatgattattccatgcactgggtccgtcaagtcccggggaagggtcttgagtgggtctctctaattagttgggatggtggtcgcacatcctatggagactctgtgaagggtcgattcaccatctccagagacaacaacaagaagaccctgtttctgcaaatgaacagtctgagacctgaggacaccgccctgtattattgtgtaaaagatactggacttcgaagctttgactcttggggccagggaattctggtcaccgtctcctcag

SEQ ID NO: 400 - DNA, 334 bp, synthetic construct
  misc_difference 8..9: note = misc_feature - n is a, c, g, or t
  source 1..334: mol_type = other DNA
gatgttgnngagactcagtctccactctccctgcccgtcacccctggagagccggcctccatctcctgcaggtctagtcagagcctcgttcatagtaatggatacacctatttggattggtacctgcagaggccagggcagtctccgcaactcctgatctatatgggttctaagcgggcctccggggtccctgacaggttcagtggcagtttctcaggcacagattttacactgaaaatcagcagagtggaggctgaagatgttggggtttattactgcatgcaagctctccaaagttggacgttcggcctagggaccaaggtggaaatcaaac

SEQ ID NO: 401 - protein, 8 aa, synthetic construct
GFTFDDYS

SEQ ID NO: 402 - protein, 8 aa, synthetic construct
ISWDGGRT

SEQ ID NO: 403 - protein, 11 aa, synthetic construct
VKDTGLRSFDS

SEQ ID NO: 404 - protein, 12 aa, synthetic construct
QSVFDDSSNKNY

SEQ ID NO: 405 - skipped

SEQ ID NO: 406 - protein, 9 aa, synthetic construct
HQYYSTPHS

SEQ ID NO: 407 - protein, 113 aa, synthetic construct
ESGGVVVQPGGSLRLSCAASGFTFDDYSMHWVRQVPGKGLEWVSLISWDGGRTSYGDSVKGRFTISRDNSKRSLFLQMTNLRPEDTAFYYCVKDTGLRSFDSWGQGTLVTVSS

SEQ ID NO: 408 - protein, 113 aa, synthetic construct
  VARIANT 2..4: note = misc_feature - Xaa can be any naturally occurring amino acid
DXXXTQSPDSVAVSLGQRATINCESSQSVFDDSSNKNYLAWYQHKPGQPPKLLIYWASSRESGVPDRFIGSGSGTDFTLTISSLQAADVAVYYCHQYYSTPHSFGQGTKVAIK

SEQ ID NO: 409 - protein, 20 aa, synthetic construct
ESGGVVVQPGGSLRLSCAAS

SEQ ID NO: 410 - protein, 17 aa, synthetic construct
MHWVRQVPGKGLEWVSL

SEQ ID NO: 411 - protein, 38 aa, synthetic construct
SYGDSVKGRFTISRDNSKRSLFLQMTNLRPEDTAFYYC

SEQ ID NO: 412 - protein, 11 aa, synthetic construct
WGQGTLVTVSS

SEQ ID NO: 413 - protein, 26 aa, synthetic construct
  VARIANT 2..4: note = misc_feature - Xaa can be any naturally occurring amino acid
DXXXTQSPDSVAVSLGQRATINCESS

SEQ ID NO: 414 - protein, 17 aa, synthetic construct
LAWYQHKPGQPPKLLIY

SEQ ID NO: 415 - protein, 36 aa, synthetic construct
SRESGVPDRFIGSGSGTDFTLTISSLQAADVAVYYC

SEQ ID NO: 416 - protein, 10 aa, synthetic construct
FGQGTKVAIK

SEQ ID NO: 417 - protein, 440 aa, synthetic construct
  VARIANT 137: note = misc_feature - Xaa can be any naturally occurring amino acid
  VARIANT 207: note = misc_feature - Xaa can be any naturally occurring amino acid
  VARIANT 351: note = misc_feature - Xaa can be any naturally occurring amino acid
ESGGVVVQPGGSLRLSCAASGFTFDDYSMHWVRQVPGKGLEWVSLISWDGGRTSYGDSVKGRFTISRDNSKRSLFLQMTNLRPEDTAFYYCVKDTGLRSFDSWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 217 aa, synthetic construct
  VARIANT 2..4: note = misc_feature - Xaa can be any naturally occurring amino acid
DXXXTQSPDSVAVSLGQRATINCESSQSVFDDSSNKNYLAWYQHKPGQPPKLLIYWASSRESGVPDRFIGSGSGTDFTLTISSLQAADVAVYYCHQYYSTPHSFGQGTKVAIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 419 - DNA, 342 bp, synthetic construct
  source 1..342: mol_type = other DNA
tggagtctggcggagtcgtggtacagcctggggggtccctgagactctcctgtgcagcctctggattcacctttgatgattattccatgcactgggtccgtcaagttccggggaagggtcttgagtgggtctctcttattagttgggatggtggccggacatcctatggagactcagtgaagggtcgattcaccatctccagagacaacagcaagaggtccctgtttctgcaaatgaccaatctgagacctgaagacaccgccttctattactgtgtaaaagatactggactccgaagttttgactcttggggccagggaaccctggtcaccgtctcctcag

SEQ ID NO: 420 - DNA, 340 bp, synthetic construct
  misc_difference 6..7: note = misc_feature - n is a, c, g, or t
  misc_difference 9..10: note = misc_feature - n is a, c, g, or t
  source 1..340: mol_type = other DNA
gacatnngnntgacccagtctccagactccgtggctgtgtctctgggccagagggccaccatcaactgcgagtccagccagagtgttttcgatgactccagcaataagaactacttagcttggtatcaacacaaaccaggacagcctcctaaactactcatttactgggcatctagccgggaatccggggtccctgaccgattcattggcagcgggtctgggacagacttcactctcaccatcagcagcctgcaggctgctgatgtggcagtttattactgtcatcaatattatagtactcctcactcttttggccaggggaccaaggtggcgattaaac

SEQ ID NO: 421 - protein, 8 aa, synthetic construct
GFTFDDYS

SEQ ID NO: 422 - protein, 8 aa, synthetic construct
ISWDGGRT

SEQ ID NO: 423 - protein, 11 aa, synthetic construct
VKDTGLRSFDY

SEQ ID NO: 424 - protein, 11 aa, synthetic construct
QGLQHSNGYNY

SEQ ID NO: 425 - skipped

SEQ ID NO: 426 - protein, 8 aa, synthetic construct
MQALQSWT

SEQ ID NO: 427 - protein, 113 aa, synthetic construct
ESGGVVVQPGGSLRLSCAASGFTFDDYSMHWVRQLPGKGLEWVSLISWDGGRTDYADSVKGRFTISRDNSKSSLLLQMNSLRLEDTALYYCVKDTGLRSFDYWGQGTLVTVSS

SEQ ID NO: 428 - protein, 111 aa, synthetic construct
DVGETQSPLSLPVTPGEPASMSCRSSQGLQHSNGYNYVDWYLQKPGQSPQLLIYMGSKRASGVPDRFSGSGSGTDFTLKISRVEAEDVGVYYCMQALQSWTFGQGTKVEIK

SEQ ID NO: 429 - protein, 20 aa, synthetic construct
ESGGVVVQPGGSLRLSCAAS

SEQ ID NO: 430 - protein, 17 aa, synthetic construct
MHWVRQLPGKGLEWVSL

SEQ ID NO: 431 - protein, 38 aa, synthetic construct
DYADSVKGRFTISRDNSKSSLLLQMNSLRLEDTALYYC

SEQ ID NO: 432 - protein, 11 aa, synthetic construct
WGQGTLVTVSS

SEQ ID NO: 433 - protein, 26 aa, synthetic construct
DVGETQSPLSLPVTPGEPASMSCRSS

SEQ ID NO: 434 - protein, 17 aa, synthetic construct
VDWYLQKPGQSPQLLIY

SEQ ID NO: 435 - protein, 36 aa, synthetic construct; identical to SEQ ID NO: 275

SEQ ID NO: 436 - protein, 10 aa, synthetic construct
FGQGTKVEIK

SEQ ID NO: 437 - protein, 440 aa, synthetic construct
  VARIANT 137: note = misc_feature - Xaa can be any naturally occurring amino acid
  VARIANT 207: note = misc_feature - Xaa can be any naturally occurring amino acid
  VARIANT 351: note = misc_feature - Xaa can be any naturally occurring amino acid
ESGGVVVQPGGSLRLSCAASGFTFDDYSMHWVRQLPGKGLEWVSLISWDGGRTDYADSVKGRFTISRDNSKSSLLLQMNSLRLEDTALYYCVKDTGLRSFDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 215 aa, synthetic construct
DVGETQSPLSLPVTPGEPASMSCRSSQGLQHSNGYNYVDWYLQKPGQSPQLLIYMGSKRASGVPDRFSGSGSGTDFTLKISRVEAEDVGVYYCMQALQSWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 439 - DNA, 341 bp, synthetic construct
  source 1..341: mol_type = other DNA
ggagtctggcggagtcgtggtacagcctgggggatccctgagactctcctgtgcagcctctggattcacttttgatgattactccatgcactgggtccgtcaacttccggggaaaggtctggagtgggtctctcttattagttgggatggaggtcgcacagactatgcagactctgtgaagggtcgattcaccatctccagagacaatagcaagagctccctgcttctgcaaatgaacagtctgagacttgaagacaccgccttgtattactgtgtaaaagatacagggcttcgaagctttgactactggggccagggaaccctggtcaccgtctcctcag

SEQ ID NO: 440 - DNA, 334 bp, synthetic construct
  misc_difference 9: note = misc_feature - n is a, c, g, or t
  source 1..334: mol_type = other DNA
gatgttggngagactcagtctccactctccctgcccgtcacccctggagagccggcctccatgtcctgcaggtctagtcagggcctccagcatagtaatggatacaactatgtggattggtacctgcagaagccagggcagtctccacagctcctgatctatatgggctctaagcgggcctccggggtccctgacaggttcagtggcagtggatcaggcacagattttacactgaaaatcagcagagtggaggctgaggatgttggggtttattactgcatgcaagctctacaaagttggacgttcggccaagggaccaaggtggaaatcaaac

SEQ ID NO: 441 - protein, 8 aa, synthetic construct
GFTFNYAV

SEQ ID NO: 442 - protein, 10 aa, synthetic construct
IDSESDGGTT

SEQ ID NO: 443 - protein, 15 aa, synthetic construct
NTHFYCTTTSCYGDH

SEQ ID NO: 444 - protein, 12 aa, synthetic construct
QSVLYSPKNKHY

SEQ ID NO: 445 - skipped

SEQ ID NO: 446 - protein, 8 aa, synthetic construct
QQYYSGRT

SEQ ID NO: 447 - protein, 119 aa, synthetic construct
ESGGGLVKPGESLRLSCEGSGFTFNYAVMSWVRQAPGKGLEWVGRIDSESDGGTTDYAAPVKGRFTISRDDSRNTLHLQMNSLEIGDTAVYYCNTHFYCTTTSCYGDHWGQGTLVTVSS

SEQ ID NO: 448 - protein, 112 aa, synthetic construct
  VARIANT 2..3: note = misc_feature - Xaa can be any naturally occurring amino acid
TXXVTQSPEFLSLSLGERASINCKASQSVLYSPKNKHYLAWYQQKPGQPPKLLMYWASTRESGVPDRFSGSGSGTDFTLTINTFQAEDVAVYYCQQYYSGRTFGPGTKVDIK

SEQ ID NO: 449 - protein, 20 aa, synthetic construct
ESGGGLVKPGESLRLSCEGS

SEQ ID NO: 450 - protein, 17 aa, synthetic construct
MSWVRQAPGKGLEWVGR

SEQ ID NO: 451 - protein, 38 aa, synthetic construct
DYAAPVKGRFTISRDDSRNTLHLQMNSLEIGDTAVYYC

SEQ ID NO: 452 - protein, 11 aa, synthetic construct
WGQGTLVTVSS

SEQ ID NO: 453 - protein, 26 aa, synthetic construct
  VARIANT 2..3: note = misc_feature - Xaa can be any naturally occurring amino acid
TXXVTQSPEFLSLSLGERASINCKAS

SEQ ID NO: 454 - protein, 17 aa, synthetic construct
LAWYQQKPGQPPKLLMY

SEQ ID NO: 455 - protein, 36 aa, synthetic construct
TRESGVPDRFSGSGSGTDFTLTINTFQAEDVAVYYC

SEQ ID NO: 456 - protein, 10 aa, synthetic construct
FGPGTKVDIK

SEQ ID NO: 457 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
ESGGGLVKPGESLRLSCEGSGFTFNYAVMSWVRQAPGKGLEWVGRIDSESDGGTTDYAAPVKGRFTISRDDSRNTLHLQMNSLEIGDTAVYYCNTHFYCTTTSCYGDHWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - protein, 216 aa, synthetic construct
  VARIANT 2..3: note = misc_feature - Xaa can be any naturally occurring amino acid
TXXVTQSPEFLSLSLGERASINCKASQSVLYSPKNKHYLAWYQQKPGQPPKLLMYWASTRESGVPDRFSGSGSGTDFTLTINTFQAEDVAVYYCQQYYSGRTFGPGTKVDIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 459 - DNA, 358 bp, synthetic construct
  source 1..358: mol_type = other DNA
gagtctgggggaggcttggtaaagcctggggagtcccttcgactctcctgtgaaggctctggattcactttcaattacgccgtgatgagctgggtccgccaggctccagggaaggggctggagtgggttggccgtattgatagcgaaagtgacggtgggacaacagactacgctgcacccgtgaaaggcagattcaccatctcaagagacgattcaagaaacacgctgcatctgcagatgaacagcctggaaatcggcgacacagccgtttattattgtaacacccacttctactgtacgacaaccagctgctatggagaccattggggccaggggaccctggtcaccgtctcctcag

SEQ ID NO: 460 - DNA, 337 bp, synthetic construct
  misc_difference 5: note = misc_feature - n is a, c, g, or t
  misc_difference 8..9: note = misc_feature - n is a, c, g, or t
  source 1..337: mol_type = other DNA
acatnggnngtgacccagtctccagagttcctgagtctgtctctgggcgagagggcctccatcaactgcaaggccagccagagtgttttatacagccccaaaaataagcactacttggcttggtatcagcagaaacctggacagcctcctaagctgctcatgtattgggcttctacccgggaatccggggtccctgaccgattcagtggcagcgggtctggtacagatttcactctcaccatcaacaccttccaggctgaggatgtggcagtctattactgtcagcaatactatagtggtcgcactttcggccctgggaccaaagtggatatcaaac

SEQ ID NO: 461 - protein, 8 aa, synthetic construct
GYSFTRFD

SEQ ID NO: 462 - protein, 8 aa, synthetic construct
MNPKSGHS

SEQ ID NO: 463 - protein, 8 aa, synthetic construct
  VARIANT 8: note = misc_feature - Xaa can be any naturally occurring amino acid
ARGVDNRX

SEQ ID NO: 464 - protein, 12 aa, synthetic construct
QSVFDDSSNKNY

SEQ ID NO: 465 - skipped

SEQ ID NO: 466 - protein, 9 aa, synthetic construct
LQYYSTPHS

SEQ ID NO: 467 - protein, 109 aa, synthetic construct
  VARIANT 98: note = misc_feature - Xaa can be any naturally occurring amino acid
SGAEVKKPGASVKVSCKTSGYSFTRFDINWVRQATGQGLEWMGWMNPKSGHSGPAQKFQGRITMTVNTSISTAYMELSSLRFEDTAVYYCARGVDNRXWGQGTLITVSS

SEQ ID NO: 468 - protein, 113 aa, synthetic construct
  VARIANT 3..4: note = misc_feature - Xaa can be any naturally occurring amino acid
DIXXTQSPDSVAVSLGQRATINCESSQSVFDDSSNKNYLAWYQHKPGQPPKLLIYWASSRESGVPDRFIGSGSGTDFTLTISSLQAADVAVYYCLQYYSTPHSFGQGTKVAIN

SEQ ID NO: 469 - protein, 19 aa, synthetic construct
SGAEVKKPGASVKVSCKTS

SEQ ID NO: 470 - protein, 17 aa, synthetic construct
INWVRQATGQGLEWMGW

SEQ ID NO: 471 - protein, 38 aa, synthetic construct
GPAQKFQGRITMTVNTSISTAYMELSSLRFEDTAVYYC

SEQ ID NO: 472 - protein, 11 aa, synthetic construct
WGQGTLITVSS

SEQ ID NO: 473 - protein, 26 aa, synthetic construct
  VARIANT 3..4: note = misc_feature - Xaa can be any naturally occurring amino acid
DIXXTQSPDSVAVSLGQRATINCESS

SEQ ID NO: 474 - protein, 17 aa, synthetic construct
LAWYQHKPGQPPKLLIY

SEQ ID NO: 475 - protein, 36 aa, synthetic construct; identical to SEQ ID NO: 415

SEQ ID NO: 476 - protein, 10 aa, synthetic construct
FGQGTKVAIN

SEQ ID NO: 477 - protein, 436 aa, synthetic construct
  VARIANT 98: note = misc_feature - Xaa can be any naturally occurring amino acid
  VARIANT 133: note = misc_feature - Xaa can be any naturally occurring amino acid
  VARIANT 203: note = misc_feature - Xaa can be any naturally occurring amino acid
  VARIANT 347: note = misc_feature - Xaa can be any naturally occurring amino acid
SGAEVKKPGASVKVSCKTSGYSFTRFDINWVRQATGQGLEWMGWMNPKSGHSGPAQKFQGRITMTVNTSISTAYMELSSLRFEDTAVYYCARGVDNRXWGQGTLITVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 478 - protein, 217 aa, synthetic construct
  VARIANT 3..4: note = misc_feature - Xaa can be any naturally occurring amino acid
DIXXTQSPDSVAVSLGQRATINCESSQSVFDDSSNKNYLAWYQHKPGQPPKLLIYWASSRESGVPDRFIGSGSGTDFTLTISSLQAADVAVYYCLQYYSTPHSFGQGTKVAINAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 479 - DNA, 330 bp, synthetic construct
  misc_difference 294..295: note = misc_feature - n is a, c, g, or t
  source 1..330: mol_type = other DNA
agtctggggctgaggtgaagaagcctggggcctcagtgaaggtctcctgcaagacttctggatacagcttcacccgttttgatatcaactgggtgcgacaggccactggacaagggcttgagtggatgggatggatgaaccctaagagtggtcactcaggccctgcacagaagttccagggcagaatcaccatgaccgttaacacctccataagtacagcctacatggagctgagcagcctgagatttgaggacacggccgtttattattgtgcgcgaggcgtggataatcgtnnctggggccagggaaccctaatcaccgtctcctcag

SEQ ID NO: 480 - DNA, 340 bp, synthetic construct
  misc_difference 8..12: note = misc_feature - n is a, c, g, or t
  source 1..340: mol_type = other DNA
gacatcgnnnnnacccagtctccagactccgtggctgtgtctctgggccagagggccaccatcaactgcgagtccagccagagtgttttcgatgactccagcaataagaactacttagcttggtatcaacacaaaccaggacagcctcctaaactactcatttactgggcatctagccgggaatccggggtccctgaccgattcattggcagcgggtctgggacagacttcactctcaccatcagcagcctgcaggctgctgatgtggcagtttattactgtcttcaatattatagtactcctcactcttttggccaggggaccaaggtggcgattaacc

SEQ ID NO: 481 - protein, 8 aa, synthetic construct
GFTFDDYS

SEQ ID NO: 482 - protein, 8 aa, synthetic construct
ISWDGART

SEQ ID NO: 483 - protein, 11 aa, synthetic construct
VKDTGLRSFDS

SEQ ID NO: 484 - protein, 11 aa, synthetic construct
QSLLHSNGYNY

SEQ ID NO: 485 - skipped

SEQ ID NO: 486 - protein, 8 aa, synthetic construct
MQALQTWT

SEQ ID NO: 487 - protein, 114 aa, synthetic construct
VESGGVVVQPGGSLRLSCAASGFTFDDYSMHWVRQVPGKGLEWVSLISWDGARTSYGDSVKGRFTISRDNSKRSLFLQMTNLRPEDTAFYYCVKDTGLRSFDSWGQGTLVTVSS

SEQ ID NO: 488 - protein, 111 aa, synthetic construct
  VARIANT 3: note = misc_feature - Xaa can be any naturally occurring amino acid
DVXETQSPLSLPVTPGEPASISCRSSQSLLHSNGYNYLDWYLQKPGQSPQLLIYMGSKRASGVPDRFSGSVSGTDFTLKISRVEAEDVGVYYCMQALQTWTFGQGTKVEIK

SEQ ID NO: 489 - protein, 21 aa, synthetic construct
VESGGVVVQPGGSLRLSCAAS

SEQ ID NO: 490 - protein, 17 aa, synthetic construct
MHWVRQVPGKGLEWVSL

SEQ ID NO: 491 - protein, 38 aa, synthetic construct; identical to SEQ ID NO: 411

SEQ ID NO: 492 - protein, 11 aa, synthetic construct
WGQGTLVTVSS

SEQ ID NO: 493 - protein, 26 aa, synthetic construct
  VARIANT 3: note = misc_feature - Xaa can be any naturally occurring amino acid
DVXETQSPLSLPVTPGEPASISCRSS

SEQ ID NO: 494 - protein, 17 aa, synthetic construct
LDWYLQKPGQSPQLLIY

SEQ ID NO: 495 - protein, 36 aa, synthetic construct
KRASGVPDRFSGSVSGTDFTLKISRVEAEDVGVYYC

SEQ ID NO: 496 - protein, 10 aa, synthetic construct
FGQGTKVEIK

SEQ ID NO: 497 - protein, 441 aa, synthetic construct
  VARIANT 138: note = misc_feature - Xaa can be any naturally occurring amino acid
  VARIANT 208: note = misc_feature - Xaa can be any naturally occurring amino acid
  VARIANT 352: note = misc_feature - Xaa can be any naturally occurring amino acid
VESGGVVVQPGGSLRLSCAASGFTFDDYSMHWVRQVPGKGLEWVSLISWDGARTSYGDSVKGRFTISRDNSKRSLFLQMTNLRPEDTAFYYCVKDTGLRSFDSWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 498 - protein, 215 aa, synthetic construct
  VARIANT 3: note = misc_feature - Xaa can be any naturally occurring amino acid
DVXETQSPLSLPVTPGEPASISCRSSQSLLHSNGYNYLDWYLQKPGQSPQLLIYMGSKRASGVPDRFSGSVSGTDFTLKISRVEAEDVGVYYCMQALQTWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 499 - DNA, 343 bp, synthetic construct
  source 1..343: mol_type = other DNA
gtggagtctggcggagtcgtggtacagcctggggggtccctgagactctcctgtgcagcctctggattcacctttgatgattattccatgcactgggtccgtcaagttccggggaagggtcttgagtgggtctctcttattagttgggatggtgcccggacatcctatggagactctgtgaagggtcgattcaccatctccagagacaacagcaagaggtccctgtttctgcaaatgaccaatctgagacctgaagacaccgccttctattactgtgtaaaagatactggacttcgaagttttgactcttggggccagggaaccctggtcaccgtctcctcag

SEQ ID NO: 500 - DNA, 334 bp, synthetic construct
  misc_difference 7..9: note = misc_feature - n is a, c, g, or t
  source 1..334: mol_type = other DNA
gatgttnnngagactcagtctccactctccctgcccgtcacccctggagagccggcctccatctcctgcaggtctagtcagagcctcctgcatagtaatggatacaactatttggattggtacctccagaagccagggcagtctccacagctcctgatctatatgggttctaagcgggcctccggggtccctgacaggttcagtggcagtgtctcaggcacagattttacactgaaaatcagcagagtggaggctgaggatgttggggtttattactgcatgcaagctctacaaacttggacgttcggccaagggaccaaggtggaaatcaaac

SEQ ID NO: 501 - protein, 8 aa, synthetic construct
GFTFGDYT

SEQ ID NO: 502 - protein, 8 aa, synthetic construct
IKWNGGGI

SEQ ID NO: 503 - protein, 11 aa, synthetic construct
AKDIGLRSLDS

SEQ ID NO: 504 - protein, 11 aa, synthetic construct
QSLLHSNGYTY

SEQ ID NO: 505 - skipped

SEQ ID NO: 506 - protein, 8 aa, synthetic construct
MQALQGWT

SEQ ID NO: 507 - protein, 117 aa, synthetic construct
  VARIANT 2..3: note = misc_feature - Xaa can be any naturally occurring amino acid
  VARIANT 63: note = misc_feature - Xaa can be any naturally occurring amino acid
CXXVESGGGLVQPGRSLRLSCTASGFTFGDYTMHWVRQAPGKGLEWVSNIKWNGGGIGYADSXKGRFTISRDNAGNSLYLQMNSLRPEDTAFYYCAKDIGLRSLDSWGQGTLVIVSS

SEQ ID NO: 508 - protein, 111 aa, synthetic construct
DVVMTQSPLSLPVTPGEPASISCRSSQSLLHSNGYTYLDWYLQKPGQSPQLLIYMGSKRASGVPDRFSGSFSDTDFTLTISRVEAEDVGVYYCMQALQGWTFGQGTKVEIK

SEQ ID NO: 509 - protein, 24 aa, synthetic construct
  VARIANT 2..3: note = misc_feature - Xaa can be any naturally occurring amino acid
CXXVESGGGLVQPGRSLRLSCTAS

SEQ ID NO: 510 - protein, 17 aa, synthetic construct
MHWVRQAPGKGLEWVSN

SEQ ID NO: 511 - protein, 38 aa, synthetic construct
  VARIANT 6: note = misc_feature - Xaa can be any naturally occurring amino acid
GYADSXKGRFTISRDNAGNSLYLQMNSLRPEDTAFYYC

SEQ ID NO: 512 - protein, 11 aa, synthetic construct
WGQGTLVIVSS

SEQ ID NO: 513 - protein, 26 aa, synthetic construct
DVVMTQSPLSLPVTPGEPASISCRSS

SEQ ID NO: 514 - protein, 17 aa, synthetic construct
LDWYLQKPGQSPQLLIY

SEQ ID NO: 515 - protein, 36 aa, synthetic construct
KRASGVPDRFSGSFSDTDFTLTISRVEAEDVGVYYC

SEQ ID NO: 516 - protein, 10 aa, synthetic construct
FGQGTKVEIK

SEQ ID NO: 517 - protein, 444 aa, synthetic construct
  VARIANT 2..3: note = misc_feature - Xaa can be any naturally occurring amino acid
  VARIANT 63: note = misc_feature - Xaa can be any naturally occurring amino acid
  VARIANT 141: note = misc_feature - Xaa can be any naturally occurring amino acid
  VARIANT 211: note = misc_feature - Xaa can be any naturally occurring amino acid
  VARIANT 355: note = misc_feature - Xaa can be any naturally occurring amino acid
CXXVESGGGLVQPGRSLRLSCTASGFTFGDYTMHWVRQAPGKGLEWVSNIKWNGGGIGYADSXKGRFTISRDNAGNSLYLQMNSLRPEDTAFYYCAKDIGLRSLDSWGQGTLVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 518 - protein, 215 aa, synthetic construct
DVVMTQSPLSLPVTPGEPASISCRSSQSLLHSNGYTYLDWYLQKPGQSPQLLIYMGSKRASGVPDRFSGSFSDTDFTLTISRVEAEDVGVYYCMQALQGWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 519 - DNA, 353 bp, synthetic construct
  misc_difference 5: note = misc_feature - n is a, c, g, or t
  misc_difference 7..10: note = misc_feature - n is a, c, g, or t
  misc_difference 189: note = misc_feature - n is a, c, g, or t
  source 1..353: mol_type = other DNA
gtgcnannnngtggaatctgggggaggcttggtacagcctggcaggtccctgagactctcctgtacagcctctggtttcacctttggtgattataccatgcattgggtccggcaagctccagggaagggcctggagtgggtctcaaatattaagtggaatggtggtggcataggctatgcggactctgntaagggccgattcaccatctccagagacaacgccgggaactccctctatctacaaatgaacagtctgagacctgaggatacggccttttattactgtgcaaaagatattggactacgttcccttgactcctggggccaggggaccctggtcatcgtctcctcag

SEQ ID NO: 520 - DNA, 334 bp, synthetic construct
  source 1..334: mol_type = other DNA
gatgttgtgatgactcagtctccactctccctgcccgtcacccctggagagccggcctccatctcctgcaggtctagtcagagcctcctgcatagtaatggatacacctatttggattggtacctgcagaagccagggcagtctccacagctcctgatctatatgggttctaagcgggcctccggggtccctgacaggttcagtggcagtttctcagacacagatttcacactgacaatcagcagagtggaggctgaggatgttggggtttattactgcatgcaggctctacaaggttggacgttcggccaagggaccaaggtggaaatcaaac

SEQ ID NO: 521 - protein, 8 aa, synthetic construct
GFTFPRAW

SEQ ID NO: 522 - protein, 10 aa, synthetic construct
LKSENDGGTA

SEQ ID NO: 523 - protein, 15 aa, synthetic construct
STVPYCPGDSCYGNF

SEQ ID NO: 524 - protein, 12 aa, synthetic construct
QSLLYSSNNRNY

SEQ ID NO: 525 - skipped

SEQ ID NO: 526 - protein, 6 aa, synthetic construct
QQYWGP

SEQ ID NO: 527 - protein, 123 aa, synthetic construct
  VARIANT 2..3: note = misc_feature - Xaa can be any naturally occurring amino acid
CXXVESGGGFVKPGGSLRLSCAASGFTFPRAWMTWVRQAPGKGLEWVGRLKSENDGGTADYAEVVKDRFVITRDDSKNTLYLEMTNVKTEDTGVYFCSTVPYCPGDSCYGNFWGQGTHVIVSS

SEQ ID NO: 528 - protein, 110 aa, synthetic construct
DIVMTQSPDSLALSLGERAAINCKSSQSLLYSSNNRNYLSWYQQKPGQPPKLLIYWASTRVSGVPDRFSGSGSGTHFTLTISSLQAEDVAVYYCQQYWGPFGQGTKVEIK

SEQ ID NO: 529 - protein, 24 aa, synthetic construct
  VARIANT 2..3: note = misc_feature - Xaa can be any naturally occurring amino acid
CXXVESGGGFVKPGGSLRLSCAAS

SEQ ID NO: 530 - protein, 17 aa, synthetic construct
MTWVRQAPGKGLEWVGR

SEQ ID NO: 531 - protein, 38 aa, synthetic construct
DYAEVVKDRFVITRDDSKNTLYLEMTNVKTEDTGVYFC

SEQ ID NO: 532 - protein, 11 aa, synthetic construct
WGQGTHVIVSS

SEQ ID NO: 533 - protein, 26 aa, synthetic construct
DIVMTQSPDSLALSLGERAAINCKSS

SEQ ID NO: 534 - protein, 17 aa, synthetic construct
LSWYQQKPGQPPKLLIY

SEQ ID NO: 535 - protein, 36 aa, synthetic construct
TRVSGVPDRFSGSGSGTHFTLTISSLQAEDVAVYYC

SEQ ID NO: 536 - protein, 10 aa, synthetic construct
FGQGTKVEIK

SEQ ID NO: 537 - protein, 450 aa, synthetic construct
  VARIANT 2..3: note = misc_feature - Xaa can be any naturally occurring amino acid
  VARIANT 147: note = misc_feature - Xaa can be any naturally occurring amino acid
  VARIANT 217: note = misc_feature - Xaa can be any naturally occurring amino acid
  VARIANT 361: note = misc_feature - Xaa can be any naturally occurring amino acid
CXXVESGGGFVKPGGSLRLSCAASGFTFPRAWMTWVRQAPGKGLEWVGRLKSENDGGTADYAEVVKDRFVITRDDSKNTLYLEMTNVKTEDTGVYFCSTVPYCPGDSCYGNFWGQGTHVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 538 - protein, 214 aa, synthetic construct
DIVMTQSPDSLALSLGERAAINCKSSQSLLYSSNNRNYLSWYQQKPGQPPKLLIYWASTRVSGVPDRFSGSGSGTHFTLTISSLQAEDVAVYYCQQYWGPFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 539 - DNA, 371 bp, synthetic construct
  misc_difference 6: note = misc_feature - n is a, c, g, or t
  misc_difference 8: note = misc_feature - n is a, c, g, or t
  source 1..371: mol_type = other DNA
gtgccngntggtggagtctgggggaggcttcgtaaagcctggggggtccctcagactctcctgtgcagcctctggattcactttccctcgggcctggatgacttgggtccgccaggcgccagggaaggggctggagtgggttggccgtcttaaaagtgagaatgacggtggaacagcagactacgctgaagtcgttaaagacagatttgtcatcacgagagacgactcaaaaaatacactctacttggaaatgactaatgtgaagaccgaagacacgggcgtctatttctgtagcaccgtgccatattgtcctggtgactcctgctacggaaatttctggggccagggaacccacgtcattgtctcctcag

SEQ ID NO: 540 - DNA, 331 bp, synthetic construct
  source 1..331: mol_type = other DNA
gacatcgtgatgacccagtctccagactcgctggctctgtcactgggcgagagggccgccatcaactgcaagtccagccagagtcttttgtacagctccaataataggaattatttatcttggtatcagcagaaaccgggacagcctcctaaattgctcatttactgggcatctacccgagtatccggggtccctgaccgattcagtggcagcgggtctgggacacatttcactctcactataagcagcctgcaggctgaagatgtggcagtttattattgtcaacaatattgggggccgttcggccaagggaccaaagtggaaatcaaac

SEQ ID NO: 541 - protein, 8 aa, synthetic construct
GFTFNNYA

SEQ ID NO: 542 - protein, 8 aa, synthetic construct
ISASGGSR

SEQ ID NO: 543 - protein, 18 aa, synthetic construct
AKDRVEDGNYEKDWHFDL

SEQ ID NO: 544 - protein, 7 aa, synthetic construct
PIVSGNY

SEQ ID NO: 545 - skipped

SEQ ID NO: 546 - protein, 9 aa, synthetic construct
QQYAKSPVT

SEQ ID NO: 547 - protein, 124 aa, synthetic construct
  VARIANT 2..3: note = misc_feature - Xaa can be any naturally occurring amino acid
AXXVESGGGLGQPGGSLRLSCPASGFTFNNYAMSWIRQAPGKGLEWVSAISASGGSRFYADSVRGRFTISRDNAKNTLYLQLNSLRAEDTAVYFCAKDRVEDGNYEKDWHFDLWGRGTLVTVSS

SEQ ID NO: 548 - protein, 108 aa, synthetic construct
  VARIANT 4: note = misc_feature - Xaa can be any naturally occurring amino acid
EIVXTQSPGTLSLSPGERATLSCRASPIVSGNYLAWYQQRPGQSPRLLIYDASSRASGVPDRFSGGGSATDFTLTISRLEPEDFAVYYCQQYAKSPVTFGGGTKVEIK

SEQ ID NO: 549 - protein, 24 aa, synthetic construct
  VARIANT 2..3: note = misc_feature - Xaa can be any naturally occurring amino acid
AXXVESGGGLGQPGGSLRLSCPAS

SEQ ID NO: 550 - protein, 17 aa, synthetic construct
MSWIRQAPGKGLEWVSA

SEQ ID NO: 551 - protein, 38 aa, synthetic construct
FYADSVRGRFTISRDNAKNTLYLQLNSLRAEDTAVYFC

SEQ ID NO: 552 - protein, 11 aa, synthetic construct
WGRGTLVTVSS

SEQ ID NO: 553 - protein, 26 aa, synthetic construct
  VARIANT 4: note = misc_feature - Xaa can be any naturally occurring amino acid
EIVXTQSPGTLSLSPGERATLSCRAS

SEQ ID NO: 554 - protein, 17 aa, synthetic construct
LAWYQQRPGQSPRLLIY

SEQ ID NO: 555 - protein, 36 aa, synthetic construct
SRASGVPDRFSGGGSATDFTLTISRLEPEDFAVYYC

SEQ ID NO: 556 - protein, 10 aa, synthetic construct
FGGGTKVEIK

SEQ ID NO: 557 - protein, 451 aa, synthetic construct
  VARIANT 2..3: note = misc_feature - Xaa can be any naturally occurring amino acid
  VARIANT 148: note = misc_feature - Xaa can be any naturally occurring amino acid
  VARIANT 218: note = misc_feature - Xaa can be any naturally occurring amino acid
  VARIANT 362: note = misc_feature - Xaa can be any naturally occurring amino acid
AXXVESGGGLGQPGGSLRLSCPASGFTFNNYAMSWIRQAPGKGLEWVSAISASGGSRFYADSVRGRFTISRDNAKNTLYLQLNSLRAEDTAVYFCAKDRVEDGNYEKDWHFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 212 aa, synthetic construct
  VARIANT 4: note = misc_feature - Xaa can be any naturally occurring amino acid
EIVXTQSPGTLSLSPGERATLSCRASPIVSGNYLAWYQQRPGQSPRLLIYDASSRASGVPDRFSGGGSATDFTLTISRLEPEDFAVYYCQQYAKSPVTFGGGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 559 - DNA, 375 bp, synthetic construct
  misc_difference 8..11: note = misc_feature - n is a, c, g, or t
  source 1..375: mol_type = other DNA
gtgccaannnngtggagtcggggggaggcttggggcagccgggggggtccctgagactctcctgtccagcctctggattcacctttaacaactatgccatgagttggatccgccaggctccagggaaggggctggagtgggtctcagctattagcgccagtggcggtagcagattctacgcagactccgtgaggggccgattcaccatctccagagacaatgccaagaacaccctatatctccaactgaacagcctgagagccgaggacacggccgtatatttctgtgcgaaagatcgggtggaggacggtaactacgagaaagactggcacttcgatctctggggccgtggcaccctggtcactgtctcgtcag

SEQ ID NO: 560 - DNA, 325 bp, synthetic construct
  misc_difference 9..11: note = misc_feature - n is a, c, g, or t
  source 1..325: mol_type = other DNA
gaaattgtnnngacgcagtctccaggcaccctgtctttgtctccaggggagagagccaccctctcctgcagggccagtccgattgttagcggcaattacttagcctggtatcagcagagacctggccagtctccccggctcctcatctatgatgcatccagcagggccagtggcgtcccagacaggttcagtggcggtgggtctgcgacagacttcactctcaccatcagcagactggagcctgaagactttgcagtgtattactgtcagcagtatgctaagtcacccgtcactttcggcggagggaccaaggtggagatcaaac

SEQ ID NO: 561 - protein, 8 aa, synthetic construct
GFTFGDYT

SEQ ID NO: 562 - protein, 8 aa, synthetic construct
IKWNSADI

SEQ ID NO: 563 - protein, 11 aa, synthetic construct
VKDTGLRSFDS

SEQ ID NO: 564 - protein, 11 aa, synthetic construct
QSLLHTNGYNY

SEQ ID NO: 565 - skipped

SEQ ID NO: 566 - protein, 8 aa, synthetic construct
MQALQTWT

SEQ ID NO: 567 - protein, 114 aa, synthetic construct
  VARIANT 1: note = misc_feature - Xaa can be any naturally occurring amino acid
  VARIANT 59..60: note = misc_feature - Xaa can be any naturally occurring amino acid
XESGGGLVKPGRSLRLSCAASGFTFGDYTMHWVRQAPGKGLEWVSNIKWNSADIGYAGXXKGRFIISRDNAENFMYLQMNSLRVEDTATYFCVKDTGLRSFDSWGRGTLVIVSS

SEQ ID NO: 568 - protein, 111 aa, synthetic construct
  VARIANT 2..3: note = misc_feature - Xaa can be any naturally occurring amino acid
QXXLTQSPLSLPVTPGEPASISCRSSQSLLHTNGYNYMDWYLQKPGQSPQLLIYLGSKRASGVPDRFSGSGSGTDFTLKISRVEAEDVGVYYCMQALQTWTFGQGTKVEIK

SEQ ID NO: 569 - protein, 21 aa, synthetic construct
  VARIANT 1: note = misc_feature - Xaa can be any naturally occurring amino acid
XESGGGLVKPGRSLRLSCAAS

SEQ ID NO: 570 - protein, 17 aa, synthetic construct
MHWVRQAPGKGLEWVSN

SEQ ID NO: 571 - protein, 38 aa, synthetic construct
  VARIANT 5..6: note = misc_feature - Xaa can be any naturally occurring amino acid
GYAGXXKGRFIISRDNAENFMYLQMNSLRVEDTATYFC

SEQ ID NO: 572 - protein, 11 aa, synthetic construct
WGRGTLVIVSS

SEQ ID NO: 573 - protein, 26 aa, synthetic construct
  VARIANT 2..3: note = misc_feature - Xaa can be any naturally occurring amino acid
QXXLTQSPLSLPVTPGEPASISCRSS

SEQ ID NO: 574 - protein, 17 aa, synthetic construct
MDWYLQKPGQSPQLLIY

SEQ ID NO: 575 - protein, 36 aa, synthetic construct; identical to SEQ ID NO: 275

SEQ ID NO: 576 - protein, 10 aa, synthetic construct
FGQGTKVEIK

SEQ ID NO: 577 - protein, 441 aa, synthetic construct
  VARIANT 1: note = misc_feature - Xaa can be any naturally occurring amino acid
  VARIANT 59..60: note = misc_feature - Xaa can be any naturally occurring amino acid
  VARIANT 138: note = misc_feature - Xaa can be any naturally occurring amino acid
  VARIANT 208: note = misc_feature - Xaa can be any naturally occurring amino acid
  VARIANT 352: note = misc_feature - Xaa can be any naturally occurring amino acid
XESGGGLVKPGRSLRLSCAASGFTFGDYTMHWVRQAPGKGLEWVSNIKWNSADIGYAGXXKGRFIISRDNAENFMYLQMNSLRVEDTATYFCVKDTGLRSFDSWGRGTLVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 578 - protein, 215 aa, synthetic construct
  VARIANT 2..3: note = misc_feature - Xaa can be any naturally occurring amino acid
QXXLTQSPLSLPVTPGEPASISCRSSQSLLHTNGYNYMDWYLQKPGQSPQLLIYLGSKRASGVPDRFSGSGSGTDFTLKISRVEAEDVGVYYCMQALQTWTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 579 - DNA, 343 bp, synthetic construct
  misc_difference 2..3: note = misc_feature - n is a, c, g, or t
  misc_difference 176: note = misc_feature - n is a, c, g, or t
  source 1..343: mol_type = other DNA
gnngaatctgggggaggcttggtaaagcctggcaggtccctgagactctcctgtgcagcctctggtttcaccttcggtgattacaccatgcattgggtccggcaagctccagggaagggcctggagtgggtctcaaatattaagtggaatagcgccgacatagggtacgcgggctntkckaagggccgattcatcatctccagagacaacgccgagaacttcatgtatttacagatgaacagtctgagagttgaggacacggccacttatttctgtgtaaaagatactggactccgttcctttgactcctggggccggggaaccctggtcatcgtctcttcag

SEQ ID NO: 580 - DNA, 334 bp, synthetic construct
  misc_difference 5: note = misc_feature - n is a, c, g, or t
  misc_difference 8..9: note = misc_feature - n is a, c, g, or t
  source 1..334: mol_type = other DNA
cagtntgnnctgactcagtctccactctccctgcccgtcacccctggagagccggcctccatctcctgcaggtcgagtcagagcctccttcatactaacggctacaactatatggattggtacctgcagaagccagggcagtcaccacaactcctgatctatttgggttctaagcgggcctccggggtccctgacaggttcagtggcagtggatcaggaacagattttacactgaaaatcagcagagtggaggctgaggatgttggggtgtattactgcatgcaagctctacaaacttggacgttcggccaagggaccaaggtggaaatcaagc

SEQ ID NO: 581 - protein, 8 aa, synthetic construct
GYTFSSYD

SEQ ID NO: 582 - protein, 8 aa, synthetic construct
MNPKNGYS

SEQ ID NO: 583 - protein, 16 aa, synthetic construct
VRVRTISQGDSWYFDL

SEQ ID NO: 584 - protein, 6 aa, synthetic construct
QGLTVS

SEQ ID NO: 585 - skipped

SEQ ID NO: 586 - protein, 9 aa, synthetic construct
MHTLQPPYT

SEQ ID NO: 587 - protein, 119 aa, synthetic construct
VQSGPEVKKPGASVKVSCKASGYTFSSYDFNWVRQAPGQGLEWMGWMNPKNGYSGFAQNFRGRISMTRNSSITTAYMELTSLTSDDTAVYYCVRVRTISQGDSWYFDLWGRGTLVTVSS

SEQ ID NO: 588 - protein, 107 aa, synthetic construct
  VARIANT 62: note = misc_feature - Xaa can be any naturally occurring amino acid
DMATTQSPSSVSASEGDRAVISCRASQGLTVSLARNHPETGEAPNLLISAASSLRGTVAAPXSGSGSGTDFTLKITRVEAEDVGVYYCMHTLQPPYTFGRGTKLEIK

SEQ ID NO: 589 - protein, 21 aa, synthetic construct
VQSGPEVKKPGASVKVSCKAS

SEQ ID NO: 590 - protein, 17 aa, synthetic construct
FNWVRQAPGQGLEWMGW

SEQ ID NO: 591 - protein, 38 aa, synthetic construct
GFAQNFRGRISMTRNSSITTAYMELTSLTSDDTAVYYC

SEQ ID NO: 592 - protein, 11 aa, synthetic construct
WGRGTLVTVSS

SEQ ID NO: 593 - protein, 26 aa, synthetic construct
DMATTQSPSSVSASEGDRAVISCRAS

SEQ ID NO: 594 - protein, 17 aa, synthetic construct
LARNHPETGEAPNLLIS

SEQ ID NO: 595 - protein, 36 aa, synthetic construct
  VARIANT 10: note = misc_feature - Xaa can be any naturally occurring amino acid
SLRGTVAAPXSGSGSGTDFTLKITRVEAEDVGVYYC

SEQ ID NO: 596 - protein, 10 aa, synthetic construct
FGRGTKLEIK

SEQ ID NO: 597 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
VQSGPEVKKPGASVKVSCKASGYTFSSYDFNWVRQAPGQGLEWMGWMNPKNGYSGFAQNFRGRISMTRNSSITTAYMELTSLTSDDTAVYYCVRVRTISQGDSWYFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 598 - protein, 211 aa, synthetic construct
  VARIANT 62: note = misc_feature - Xaa can be any naturally occurring amino acid
DMATTQSPSSVSASEGDRAVISCRASQGLTVSLARNHPETGEAPNLLISAASSLRGTVAAPXSGSGSGTDFTLKITRVEAEDVGVYYCMHTLQPPYTFGRGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 599 - DNA, 359 bp, synthetic construct
  source 1..359: mol_type = other DNA
ggtgcagtctgggcctgaggtgaagaagcctggggcctcagtgaaggtctcctgcaaggcttctggatataccttcagtagttatgacttcaactgggtgcgacaggcccctggacaagggcttgagtggatgggatggatgaaccctaaaaatggttactcaggcttcgcacagaatttccggggccggatatccatgaccaggaactcctccataaccacagcctacatggagctgaccagcctgacatctgatgacacggccgtttattattgtgtgagagtgaggacaatttctcagggcgacagctggtacttcgatctctggggccgtggcaccctggtcactgtctcctcag

SEQ ID NO: 600 - DNA, 322 bp, synthetic construct
  misc_difference 185: note = misc_feature - n is a, c, g, or t
  source 1..322: mol_type = other DNA
gacatggcgacgacccagtctccatcttccgtgtctgcatctgaaggggacagagccgtcatctcttgtcgggcgagtcagggtcttaccgtgtcgttagccaggaatcacccggagacaggggaagcccctaacctcctaatctctgctgcatcgagtttgcgaggaactgtggctgcaccatncagtggcagtggatcaggcacagattttacactgaaaatcaccagagtggaggctgaggatgttggggtttattactgtatgcatactctacaacccccgtacacttttggccgggggaccaagctggagatcaaac

SEQ ID NO: 601 - protein, 8 aa, synthetic construct
GYTFSNYD

SEQ ID NO: 602 - protein, 8 aa, synthetic construct
MNPKNGYS

SEQ ID NO: 603 - protein, 16 aa, synthetic construct
VRVRTISKGDSWYFDL

SEQ ID NO: 604 - protein, 11 aa, synthetic construct
QSLLHNNGYNY

SEQ ID NO: 605 - skipped

SEQ ID NO: 606 - protein, 9 aa, synthetic construct
MHTLQPPYT

SEQ ID NO: 607 - protein, 118 aa, synthetic construct
QSGPEVKKPGASVKVSCKSSGYTFSNYDFNWVRQAPGQGLEWMGWMNPKNGYSGFAQNFRGRISMTRNSSITTAYMELTSLTSDDTALYYCVRVRTISKGDSWYFDLWGRGTLVTVSS

SEQ ID NO: 608 - protein, 112 aa, synthetic construct
  VARIANT 3..4: note = misc_feature - Xaa can be any naturally occurring amino acid
  VARIANT 68: note = misc_feature - Xaa can be any naturally occurring amino acid
  VARIANT 110: note = misc_feature - Xaa can be any naturally occurring amino acid
RCXXTQSQVSLPVTPGEPASISCRSSQSLLHNNGYNYVDWYLQKPGQSPQLLIYLVSNPVSIFRVCIXGSGSGTDFTLKITRVEAEDVGVYYCMHTLQPPYTFGQGTKLXIK

SEQ ID NO: 609 - protein, 20 aa, synthetic construct
QSGPEVKKPGASVKVSCKSS

SEQ ID NO: 610 - protein, 17 aa, synthetic construct
FNWVRQAPGQGLEWMGW

SEQ ID NO: 611 - protein, 38 aa, synthetic construct
GFAQNFRGRISMTRNSSITTAYMELTSLTSDDTALYYC

SEQ ID NO: 612 - protein, 11 aa, synthetic construct
WGRGTLVTVSS

SEQ ID NO: 613 - protein, 26 aa, synthetic construct
  VARIANT 3..4: note = misc_feature - Xaa can be any naturally occurring amino acid
RCXXTQSQVSLPVTPGEPASISCRSS

SEQ ID NO: 614 - protein, 17 aa, synthetic construct
VDWYLQKPGQSPQLLIY

SEQ ID NO: 615 - protein, 36 aa, synthetic construct
  VARIANT 11: note = misc_feature - Xaa can be any naturally occurring amino acid
NPVSIFRVCIXGSGSGTDFTLKITRVEAEDVGVYYC

SEQ ID NO: 616 - protein, 10 aa, synthetic construct
  VARIANT 8: note = misc_feature - Xaa can be any naturally occurring amino acid
FGQGTKLXIK

SEQ ID NO: 617 - protein, 445 aa, synthetic construct
  VARIANT 142: note = misc_feature - Xaa can be any naturally occurring amino acid
  VARIANT 212: note = misc_feature - Xaa can be any naturally occurring amino acid
  VARIANT 356: note = misc_feature - Xaa can be any naturally occurring amino acid
QSGPEVKKPGASVKVSCKSSGYTFSNYDFNWVRQAPGQGLEWMGWMNPKNGYSGFAQNFRGRISMTRNSSITTAYMELTSLTSDDTALYYCVRVRTISKGDSWYFDLWGRGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 618 - protein, 216 aa, synthetic construct
  VARIANT 3..4: note = misc_feature - Xaa can be any naturally occurring amino acid
  VARIANT 68: note = misc_feature - Xaa can be any naturally occurring amino acid
  VARIANT 110: note = misc_feature - Xaa can be any naturally occurring amino acid
RCXXTQSQVSLPVTPGEPASISCRSSQSLLHNNGYNYVDWYLQKPGQSPQLLIYLVSNPVSIFRVCIXGSGSGTDFTLKITRVEAEDVGVYYCMHTLQPPYTFGQGTKLXIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 619 - DNA, 356 bp, synthetic construct
  source 1..356: mol_type = other DNA
gcagtctgggcctgaggtgaagaagcctggggcctcagtgaaggtctcctgcaagtcttctggatataccttcagtaattatgacttcaactgggtgcgacaggcccctggacaagggcttgagtggatgggatggatgaaccctaaaaatggctactcaggcttcgcacagaatttccggggccggatatccatgaccaggaactcctccataaccacagcctacatggagctgaccagcctgacatctgatgacacggccctttattattgtgtgagagtgaggacaatttctaagggcgacagctggtacttcgatctctggggccgtggcaccctggtcactgtctcctcag

SEQ ID NO: 620 - DNA, 337 bp, synthetic construct
  misc_difference 9..10: note = misc_feature - n is a, c, g, or t
  misc_difference 202: note = misc_feature - n is a, c, g, or t
  misc_difference 330: note = misc_feature - n is a, c, g, or t
  source 1..337: mol_type = other DNA
cgatgttgnnagactcagtctcaagtctccctgcccgtcacccctggagagccggcctccatctcctgcaggtctagtcagagcctcctgcataacaatggatacaactatgtggattggtacctgcagaagccagggcagtctccacaactcctgatctatttggtttcgaacccagtctccatcttccgtgtctgcatcngtggcagtggatcaggcacagattttacactgaagatcaccagagtggaggctgaggatgttggggtttattactgtatgcatactctacaacccccgtacacttttggccaggggaccaagctgganatcaaac

SEQ ID NO: 621 - protein, 8 aa, synthetic construct
GFTFNNYA

SEQ ID NO: 622 - protein, 8 aa, synthetic construct
ISGRTGSA

SEQ ID NO: 623 - protein, 17 aa, synthetic construct
ARDGQEGGNYAAEYFQN

SEQ ID NO: 624 - protein, 6 aa, synthetic construct
QSISKW

SEQ ID NO: 625 - skipped

SEQ ID NO: 626 - protein, 9 aa, synthetic construct
QHFNTELLT

SEQ ID NO: 627 - protein, 120 aa, synthetic construct
VESGGGLAQPGGSLTLSCAASGFTFNNYAMSWVRQAPGKGLEWVSGISGRTGSAHYADAVRGRFTISRDNAKNTLYLQMSSLRADDTATYYCARDGQEGGNYAAEYFQNWGQGTLVTVSS

SEQ ID NO: 628 - protein, 107 aa, synthetic construct
  VARIANT 3..4: note = misc_feature - Xaa can be any naturally occurring amino acid
RHXXTQSPSTLAASVGDRVTITCRASQSISKWLAWYQQKPGKAPKLLIYKASRLQSGAPSRFSGSGSGTEFTLTINSLQPDDFATYYCQHFNTELLTFGGGTKVEIK

SEQ ID NO: 629 - protein, 21 aa, synthetic construct
VESGGGLAQPGGSLTLSCAAS

SEQ ID NO: 630 - protein, 17 aa, synthetic construct
MSWVRQAPGKGLEWVSG

SEQ ID NO: 631 - protein, 38 aa, synthetic construct
HYADAVRGRFTISRDNAKNTLYLQMSSLRADDTATYYC

SEQ ID NO: 632 - protein, 11 aa, synthetic construct
WGQGTLVTVSS

SEQ ID NO: 633 - protein, 26 aa, synthetic construct
  VARIANT 3..4: note = misc_feature - Xaa can be any naturally occurring amino acid
RHXXTQSPSTLAASVGDRVTITCRAS

SEQ ID NO: 634 - protein, 17 aa, synthetic construct
LAWYQQKPGKAPKLLIY

SEQ ID NO: 635 - protein, 36 aa, synthetic construct
RLQSGAPSRFSGSGSGTEFTLTINSLQPDDFATYYC

SEQ ID NO: 636 - protein, 10 aa, synthetic construct
FGGGTKVEIK

SEQ ID NO: 637 - protein, 447 aa, synthetic construct
  VARIANT 144: note = misc_feature - Xaa can be any naturally occurring amino acid
  VARIANT 214: note = misc_feature - Xaa can be any naturally occurring amino acid
  VARIANT 358: note = misc_feature - Xaa can be any naturally occurring amino acid
VESGGGLAQPGGSLTLSCAASGFTFNNYAMSWVRQAPGKGLEWVSGISGRTGSAHYADAVRGRFTISRDNAKNTLYLQMSSLRADDTATYYCARDGQEGGNYAAEYFQNWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 638 - protein, 211 aa, synthetic construct
  VARIANT 3..4: note = misc_feature - Xaa can be any naturally occurring amino acid
RHXXTQSPSTLAASVGDRVTITCRASQSISKWLAWYQQKPGKAPKLLIYKASRLQSGAPSRFSGSGSGTEFTLTINSLQPDDFATYYCQHFNTELLTFGGGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 639 - DNA, 361 bp, synthetic construct
  misc_difference 3: note = misc_feature - n is a, c, g, or t
  source 1..361: mol_type = other DNA
gtngagtcggggggaggcctcgcacagcctgggggatccctgacactctcctgtgcagcgtctgggttcacattcaacaattatgccatgagctgggtccgccaggctccagggaaggggctggagtgggtctcaggaataagtgggagaactggtagcgcacactacgcagacgccgtgaggggccggttcaccatctccagagacaatgccaagaacacactgtatctccaaatgagcagcctgagagccgatgacacggccacatattactgtgcgagagatggtcaagagggtggaaactacgccgctgaatactttcagaactggggccagggcaccctggtcaccgtctcctcag

SEQ ID NO: 640 - DNA, 322 bp, synthetic construct
  misc_difference 8..10: note = misc_feature - n is a, c, g, or t
  source 1..322: mol_type = other DNA
cgacatcnnnagacccagtctccttccaccctggctgcatctgtaggagacagagtcaccatcacttgccgggccagtcagagtattagtaagtggttggcctggtatcagcagaaaccagggaaagcccctaagctgctgatctataaggcatctcgtttacaaagtggggccccatcaaggttcagcggcagtggatctgggacagagttcactctcaccataaacagcctgcagcctgatgattttgcaacttattactgccaacactttaatactgaacttctcactttcggcggcgggaccaaggtggagatcaaac

SEQ ID NO: 641 - protein, 8 aa, synthetic construct
GFTFRSYA

SEQ ID NO: 642 - protein, 8 aa, synthetic construct
ISGSGGST

SEQ ID NO: 643 - protein, 17 aa, synthetic construct
AKDGQTGGNYAAEYFQH

SEQ ID NO: 644 - protein, 6 aa, synthetic construct
QSVSKW

SEQ ID NO: 645 - skipped

SEQ ID NO: 646 - protein, 9 aa, synthetic construct
QHFNTEQLT

SEQ ID NO: 647 - protein, 119 aa, synthetic construct
ESGGGLVQPGGSLRLSCAASGFTFRSYAMSWVRQAPGKGLEWVSGISGSGGSTHYADSVEGRFAVSRDNSKNTVYLEMNSLRAEDTAVYYCAKDGQTGGNYAAEYFQHWGQGSLVTVSS

SEQ ID NO: 648 - protein, 107 aa, synthetic construct
  VARIANT 4: note = misc_feature - Xaa can be any naturally occurring amino acid
RHRXTQSPSTLSASVGDRVTITCRASQSVSKWLAWYQQKAGKAPRLLIYKASTLDSGVPSRFSGSGSGTEFTLTISSLQPDDFATYYCQHFNTEQLTFGGGTKVEIK

SEQ ID NO: 649 - protein, 20 aa, synthetic construct
ESGGGLVQPGGSLRLSCAAS

SEQ ID NO: 650 - protein, 17 aa, synthetic construct
MSWVRQAPGKGLEWVSG

SEQ ID NO: 651 - protein, 38 aa, synthetic construct
HYADSVEGRFAVSRDNSKNTVYLEMNSLRAEDTAVYYC

SEQ ID NO: 652 - protein, 11 aa, synthetic construct
WGQGSLVTVSS

SEQ ID NO: 653 - protein, 26 aa, synthetic construct
  VARIANT 4: note = misc_feature - Xaa can be any naturally occurring amino acid
RHRXTQSPSTLSASVGDRVTITCRAS

SEQ ID NO: 654 - protein, 17 aa, synthetic construct
LAWYQQKAGKAPRLLIY

SEQ ID NO: 655 - protein, 36 aa, synthetic construct
TLDSGVPSRFSGSGSGTEFTLTISSLQPDDFATYYC

SEQ ID NO: 656 - protein, 10 aa, synthetic construct
FGGGTKVEIK

SEQ ID NO: 657 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
ESGGGLVQPGGSLRLSCAASGFTFRSYAMSWVRQAPGKGLEWVSGISGSGGSTHYADSVEGRFAVSRDNSKNTVYLEMNSLRAEDTAVYYCAKDGQTGGNYAAEYFQHWGQGSLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 658 - protein, 211 aa, synthetic construct
  VARIANT 4: note = misc_feature - Xaa can be any naturally occurring amino acid
RHRXTQSPSTLSASVGDRVTITCRASQSVSKWLAWYQQKAGKAPRLLIYKASTLDSGVPSRFSGSGSGTEFTLTISSLQPDDFATYYCQHFNTEQLTFGGGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 659 - DNA, 358 bp, synthetic construct
  source 1..358: mol_type = other DNA
gagtctgggggaggcttggtgcagcctggggggtccctgagactctcgtgtgcagcctctggattcacctttagaagttatgccatgagttgggtccgacaggctccagggaagggactggagtgggtctctggtattagtggtagtggcggcagcacacactatgcagactccgtggagggccggttcgccgtctccagagacaattccaagaacacggtgtatctggaaatgaacagcctgagagccgaggacacggccgtatattactgtgcgaaagatggtcaaacgggtgggaactatgccgctgagtacttccaacactggggccagggctccctggtcaccgtctcctcag

SEQ ID NO: 660 - DNA, 322 bp, synthetic construct
  misc_difference 9..11: note = misc_feature - n is a, c, g, or t
  source 1..322: mol_type = other DNA
cgacatcgnnngacccagtctccttccaccctgtctgcatctgtgggagacagagtcaccatcacttgccgggccagtcagagtgttagtaagtggttggcctggtatcagcagaaagcagggaaagcccctaggctcctgatctacaaggcgtccactttagatagtggggtcccatcaaggttcagcggcagtggatctgggacagaattcactcttaccatcagcagcctgcagcctgatgattttgcaacttattactgccaacactttaatactgaacaactcactttcggcggagggaccaaggtggagatcaaac

SEQ ID NO: 661 - protein, 8 aa, synthetic construct
GYTLTNYI

SEQ ID NO: 662 - protein, 8 aa, synthetic construct
LNPSGGQS

SEQ ID NO: 663 - protein, 20 aa, synthetic construct
ARVHCTSTSCPYYYYYGLDV

SEQ ID NO: 664 - protein, 6 aa, synthetic construct
QGLNSW

SEQ ID NO: 665 - skipped

SEQ ID NO: 666 - protein, 9 aa, synthetic construct
QQANSFPFT

SEQ ID NO: 667 - protein, 123 aa, synthetic construct
  VARIANT 67: note = misc_feature - Xaa can be any naturally occurring amino acid
  VARIANT 72..74: note = misc_feature - Xaa can be any naturally occurring amino acid
  VARIANT 77: note = misc_feature - Xaa can be any naturally occurring amino acid
  VARIANT 84..85: note = misc_feature - Xaa can be any naturally occurring amino acid
VQSGTEVKQPGASVRVSCRASGYTLTNYIIHWVRQAPGQGLEWMGTLNPSGGQSNYAQKFQGRITIXRDTSXXXVYXDISSLTXXDTAVYYCARVHCTSTSCPYYYYYGLDVWGQGTTVTVSS

SEQ ID NO: 668 - protein, 107 aa, synthetic construct
  VARIANT 2..4: note = misc_feature - Xaa can be any naturally occurring amino acid
DXXXTQSPSSVSASVGDRVTITCRASQGLNSWLAWYQQKPGRAPKLLIHSVSALQSGVPSRFSGSRSGTDFTLTISSLQPEDFATYYCQQANSFPFTFGPGTRVDVN

SEQ ID NO: 669 - protein, 21 aa, synthetic construct
VQSGTEVKQPGASVRVSCRAS

SEQ ID NO: 670 - protein, 17 aa, synthetic construct
IHWVRQAPGQGLEWMGT

SEQ ID NO: 671 - protein, 38 aa, synthetic construct
  VARIANT 13: note = misc_feature - Xaa can be any naturally occurring amino acid
  VARIANT 18..20: note = misc_feature - Xaa can be any naturally occurring amino acid
  VARIANT 23: note = misc_feature - Xaa can be any naturally occurring amino acid
  VARIANT 30..31: note = misc_feature - Xaa can be any naturally occurring amino acid
NYAQKFQGRITIXRDTSXXXVYXDISSLTXXDTAVYYC

SEQ ID NO: 672 - protein, 11 aa, synthetic construct
WGQGTTVTVSS

SEQ ID NO: 673 - protein, 26 aa, synthetic construct
  VARIANT 2..4: note = misc_feature - Xaa can be any naturally occurring amino acid
DXXXTQSPSSVSASVGDRVTITCRAS

SEQ ID NO: 674 - protein, 17 aa, synthetic construct
LAWYQQKPGRAPKLLIH

SEQ ID NO: 675 - protein, 36 aa, synthetic construct
ALQSGVPSRFSGSRSGTDFTLTISSLQPEDFATYYC

SEQ ID NO: 676 - protein, 10 aa, synthetic construct
FGPGTRVDVN

SEQ ID NO: 677 - protein, 450 aa, synthetic construct
  VARIANT 67: note = misc_feature - Xaa can be any naturally occurring amino acid
  VARIANT 72..74: note = misc_feature - Xaa can be any naturally occurring amino acid
  VARIANT 77: note = misc_feature - Xaa can be any naturally occurring amino acid
  VARIANT 84..85: note = misc_feature - Xaa can be any naturally occurring amino acid
  VARIANT 147: note = misc_feature - Xaa can be any naturally occurring amino acid
  VARIANT 217: note = misc_feature - Xaa can be any naturally occurring amino acid
  VARIANT 361: note = misc_feature - Xaa can be any naturally occurring amino acid
VQSGTEVKQPGASVRVSCRASGYTLTNYIIHWVRQAPGQGLEWMGTLNPSGGQSNYAQKFQGRITIXRDTSXXXVYXDISSLTXXDTAVYYCARVHCTSTSCPYYYYYGLDVWGQGTTVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 211 aa, synthetic construct
  VARIANT 2..4: note = misc_feature - Xaa can be any naturally occurring amino acid
DXXXTQSPSSVSASVGDRVTITCRASQGLNSWLAWYQQKPGRAPKLLIHSVSALQSGVPSRFSGSRSGTDFTLTISSLQPEDFATYYCQQANSFPFTFGPGTRVDVNAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 679 - DNA, 369 bp, synthetic construct
  source 1..369: mol_type = other DNA
gtgcagtctgggactgaagtgaagcagcccggggcctcagtgagggtttcctgcagggcatccggatacaccttaaccaactacataatacactgggtgcgacaggcccctggacaagggcttgagtggatgggaacactcaaccctagtggtggtcagtcaaattacgcacagaagttccagggaagaatcaccatcwccagrgacacstccmygarswcagtgtacmtggacatcagcagcctgacatrtgrggacacggctgtgtattactgtgcgagagtccattgtactagtaccagctgcccctattactactactacggtttggacgtctggggccaagggaccacggtcaccgtctcctca

SEQ ID NO: 680 - DNA, 322 bp, synthetic construct
  misc_difference 6..8: note = misc_feature - n is a, c, g, or t
  misc_difference 10: note = misc_feature - n is a, c, g, or t
  source 1..322: mol_type = other DNA
gacatnnntnagacccagtctccatcttccgtgtctgcatctgttggagacagagtcaccatcacttgtcgggcgagccagggtcttaacagctggttagcctggtatcagcaaaaaccagggagagcccctaagctcctgattcattctgtttcggctttgcaaagtggagtcccatccaggttcagcggcagtagatctgggacagatttcactctcaccatcagcagcctgcagcctgaagattttgccacttactattgtcagcaggctaacagtttcccattcactttcggccctgggaccagagtggatgtcaacc

SEQ ID NO: 681 - protein, 8 aa, synthetic construct
GYTFIDYY

SEQ ID NO: 682 - protein, 8 aa, synthetic construct
INPKSGGT

SEQ ID NO: 683 - protein, 15 aa, synthetic construct
ARPRRRGNYSYGMDV

SEQ ID NO: 684 - protein, 6 aa, synthetic construct
QNIDDY

SEQ ID NO: 685 - skipped

SEQ ID NO: 686 - protein, 9 aa, synthetic construct
QETYTFPYT

SEQ ID NO: 687 - protein, 115 aa, synthetic construct
GAEVAKPGASVKVSCKASGYTFIDYYIYWVRQAPGQGLKSVAWINPKSGGTNYAQDFQGRVTLTRDTSITTAYMELNGLRSDDTAVYYCARPRRRGNYSYGMDVWGQGTAVIVSS

SEQ ID NO: 688 - protein, 107 aa, synthetic construct
  VARIANT 3: note = misc_feature - Xaa can be any naturally occurring amino acid
RHXVTQSPSSLSASVGDRVTITCRASQNIDDYLNWYQQTEGKAPQLLIFGASSLHNGVPTRFSGSGSGRDFTLTITSLQRDDFATYYCQETYTFPYTFGQGTKLEIK

SEQ ID NO: 689 - protein, 18 aa, synthetic construct
GAEVAKPGASVKVSCKAS

SEQ ID NO: 690 - protein, 17 aa, synthetic construct
IYWVRQAPGQGLKSVAW

SEQ ID NO: 691 - protein, 38 aa, synthetic construct
NYAQDFQGRVTLTRDTSITTAYMELNGLRSDDTAVYYC

SEQ ID NO: 692 - protein, 11 aa, synthetic construct
WGQGTAVIVSS

SEQ ID NO: 693 - protein, 26 aa, synthetic construct
  VARIANT 3: note = misc_feature - Xaa can be any naturally occurring amino acid
RHXVTQSPSSLSASVGDRVTITCRAS

SEQ ID NO: 694 - protein, 17 aa, synthetic construct
LNWYQQTEGKAPQLLIF

SEQ ID NO: 695 - protein, 36 aa, synthetic construct
SLHNGVPTRFSGSGSGRDFTLTITSLQRDDFATYYC

SEQ ID NO: 696 - protein, 10 aa, synthetic construct
FGQGTKLEIK

SEQ ID NO: 697 - protein, 442 aa, synthetic construct
  VARIANT 139: note = misc_feature - Xaa can be any naturally occurring amino acid
  VARIANT 209: note = misc_feature - Xaa can be any naturally occurring amino acid
  VARIANT 353: note = misc_feature - Xaa can be any naturally occurring amino acid
GAEVAKPGASVKVSCKASGYTFIDYYIYWVRQAPGQGLKSVAWINPKSGGTNYAQDFQGRVTLTRDTSITTAYMELNGLRSDDTAVYYCARPRRRGNYSYGMDVWGQGTAVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 698 - protein, 211 aa, synthetic construct
  VARIANT 3: note = misc_feature - Xaa can be any naturally occurring amino acid
RHXVTQSPSSLSASVGDRVTITCRASQNIDDYLNWYQQTEGKAPQLLIFGASSLHNGVPTRFSGSGSGRDFTLTITSLQRDDFATYYCQETYTFPYTFGQGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 699 - DNA, 347 bp, synthetic construct
  source 1..347: mol_type = other DNA
ctggggctgaggtggcgaagcctggggcctcagtgaaggtctcctgcaaggcgtctggatacaccttcatcgactactatatatactgggtgcgacaggcccctggacaaggtcttaaatcggtggcatggatcaaccctaagagtggtggcacaaactacgcacaggactttcagggcagggtcacccttaccagggacacgtccatcaccacagcctacatggagctgaacggtctgagatctgacgacacggccgtttattactgtgcgagaccgagacgacggggaaattattcctacggcatggacgtctggggccaagggaccgctgtcattgtctcctca

SEQ ID NO: 700 - DNA, 322 bp, synthetic construct
  misc_difference 7: note = misc_feature - n is a, c, g, or t
  source 1..322: mol_type = other DNA
cgacatngtgtgacccagtctccatcctccctgtctgcatctgtaggagacagagtcaccatcacttgccgggcaagtcagaacattgacgactatttaaactggtatcagcagacagaagggaaagcccctcagctcctgatctttggtgcatcgagtttgcacaatggggtcccaacaaggttcagtggcagtggatctgggagagacttcactctcaccatcaccagtctgcaacgtgacgattttgcaacttactactgtcaagagacttacactttcccatacacttttggccaggggaccaagctggagatcaaac

SEQ ID NO: 701 - protein, 8 aa, synthetic construct
GFTFSKAW

SEQ ID NO: 702 - protein, 10 aa, synthetic construct
IKSNIDDGTI

SEQ ID NO: 703 - protein, 15 aa, synthetic construct
FTVPYCNGTHCYGTA

SEQ ID NO: 704 - protein, 12 aa, synthetic construct
QSLFYSGTNKNY

SEQ ID NO: 705 - skipped

SEQ ID NO: 706 - protein, 6 aa, synthetic construct
  VARIANT 4: note = misc_feature - Xaa can be any naturally occurring amino acid
QRHXET

SEQ ID NO: 707 - protein, 124 aa, synthetic construct
RCQLVESGGSLVKPGGSLRLSCAASGFTFSKAWMSWARQAPGKGLEWVGRIKSNIDDGTIDYAASVKGRFTIARDDSENTLHLQMNSLRTEDTAVYYCFTVPYCNGTHCYGTAWGQGTLVIVSS

SEQ ID NO: 708 - protein, 110 aa, synthetic construct
  VARIANT 47..48: note = misc_feature - Xaa can be any naturally occurring amino acid
  VARIANT 74..76: note = misc_feature - Xaa can be any naturally occurring amino acid
  VARIANT 87: note = misc_feature - Xaa can be any naturally occurring amino acid
  VARIANT 89..92: note = misc_feature - Xaa can be any naturally occurring amino acid
  VARIANT 98: note = misc_feature - Xaa can be any naturally occurring amino acid
  VARIANT 102: note = misc_feature - Xaa can be any naturally occurring amino acid
DIVMTQSPDSLAVSLGERATINCKSSQSLFYSGTNKNYLELWLHHLXXPPKLLIYWASIRESGVPDRFSGCGDXXXSRLPISSLRAXDXXXXYCQRHXETFXQGTRVEIK

SEQ ID NO: 709 - protein, 25 aa, synthetic construct
RCQLVESGGSLVKPGGSLRLSCAAS

SEQ ID NO: 710 - protein, 17 aa, synthetic construct
MSWARQAPGKGLEWVGR

SEQ ID NO: 711 - protein, 38 aa, synthetic construct
DYAASVKGRFTIARDDSENTLHLQMNSLRTEDTAVYYC

SEQ ID NO: 712 - protein, 11 aa, synthetic construct
WGQGTLVIVSS

SEQ ID NO: 713 - protein, 26 aa, synthetic construct
DIVMTQSPDSLAVSLGERATINCKSS

SEQ ID NO: 714 - protein, 17 aa, synthetic construct
  VARIANT 9..10: note = misc_feature - Xaa can be any naturally occurring amino acid
LELWLHHLXXPPKLLIY

SEQ ID NO: 715 - protein, 36 aa, synthetic construct
  VARIANT 16..18: note = misc_feature - Xaa can be any naturally occurring amino acid
  VARIANT 29: note = misc_feature - Xaa can be any naturally occurring amino acid
  VARIANT 31..34: note = misc_feature - Xaa can be any naturally occurring amino acid
IRESGVPDRFSGCGDXXXSRLPISSLRAXDXXXXYC

SEQ ID NO: 716 - protein, 10 aa, synthetic construct
  VARIANT 2: note = misc_feature - Xaa can be any naturally occurring amino acid
FXQGTRVEIK

SEQ ID NO: 717 - protein, 451 aa, synthetic construct
  VARIANT 148: note = misc_feature - Xaa can be any naturally occurring amino acid
  VARIANT 218: note = misc_feature - Xaa can be any naturally occurring amino acid
  VARIANT 362: note = misc_feature - Xaa can be any naturally occurring amino acid
RCQLVESGGSLVKPGGSLRLSCAASGFTFSKAWMSWARQAPGKGLEWVGRIKSNIDDGTIDYAASVKGRFTIARDDSENTLHLQMNSLRTEDTAVYYCFTVPYCNGTHCYGTAWGQGTLVIVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 718 - protein, 214 aa, synthetic construct
  VARIANT 47..48: note = misc_feature - Xaa can be any naturally occurring amino acid
  VARIANT 74..76: note = misc_feature - Xaa can be any naturally occurring amino acid
  VARIANT 87: note = misc_feature - Xaa can be any naturally occurring amino acid
  VARIANT 89..92: note = misc_feature - Xaa can be any naturally occurring amino acid
  VARIANT 98: note = misc_feature - Xaa can be any naturally occurring amino acid
  VARIANT 102: note = misc_feature - Xaa can be any naturally occurring amino acid
DIVMTQSPDSLAVSLGERATINCKSSQSLFYSGTNKNYLELWLHHLXXPPKLLIYWASIRESGVPDRFSGCGDXXXSRLPISSLRAXDXXXXYCQRHXETFXQGTRVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 719 - DNA, 373 bp, synthetic construct
  source 1..373: mol_type = other DNA
aggtgccagctggtggagtctgggggatccttggtgaagcctggggggtccctcagactctcctgtgcagcctctggattcactttcagtaaggcctggatgagttgggcccgccaggctccagggaagggtctggagtgggtcggccgtattaaaagtaatattgatgatgggactatagactacgctgcatccgtgaaaggaagattcaccatcgcaagagatgattccgaaaacacactccacctccaaatgaatagcctgagaaccgaggacacagccgtttattactgtttcacagttccctattgtaatgggactcactgttatggaactgcgtggggccagggaaccctggtcatcgtctcgtcag

SEQ ID NO: 720 - DNA, 331 bp, synthetic construct
  misc_difference 138..139: note = misc_feature - n is a, c, g, or t
  source 1..331: mol_type = other DNA
gacatcgtgatgacccagtctccagactccctggctgtgtctctgggcgagagggccaccatcaactgcaagtccagccagagtcttttctacagcggcaccaataaaaactacttagaactgtggctgcaccatctnngtcwgcctcctaagttgctcatttactgggcttctatccgggagtccggggtccctgaccgattcagtggctgcggtgatgrsmcagwytccagactccctatcagcagcctgcgggctgragatgkgscastttmwtactgtcarcggcattgrgagacgttckgccaagggaccagggttgagatcaaac

SEQ ID NO: 721 - protein, 8 aa, synthetic construct
GFTFSDYY

SEQ ID NO: 722 - protein, 10 aa, synthetic construct
SRNKANSYTT

SEQ ID NO: 723 - protein, 13 aa, synthetic construct
ARAFSGGSYCSDY

SEQ ID NO: 724 - protein, 6 aa, synthetic construct
QGISSY

SEQ ID NO: 725 - skipped

SEQ ID NO: 726 - protein, 9 aa, synthetic construct
QQLNSYPST

SEQ ID NO: 727 - protein, 121 aa, synthetic construct
  VARIANT 2..3: note = misc_feature - Xaa can be any naturally occurring amino acid
CXXVESGGGLVQPGGSLRLSCAASGFTFSDYYMDWVRQAPGKGLEWVGRSRNKANSYTTEYAASVKGRFTISRDDSKNSVYLQMNSLKTEDTAVYYCARAFSGGSYCSDYWGQGTLVTVSS

SEQ ID NO: 728 - protein, 107 aa, synthetic construct
  VARIANT 4: note = misc_feature - Xaa can be any naturally occurring amino acid
DIPXTQSPSFLSASVGDRVTITCRTSQGISSYLAWYQQKPGKAPKLLIYAASTLQSGVPSRFSGSGSGTEFTLTISSLQPEDFATYYCQQLNSYPSTFGQGTRLEIK

SEQ ID NO: 729 - protein, 24 aa, synthetic construct
  VARIANT 2..3: note = misc_feature - Xaa can be any naturally occurring amino acid
CXXVESGGGLVQPGGSLRLSCAAS

SEQ ID NO: 730 - protein, 17 aa, synthetic construct
MDWVRQAPGKGLEWVGR

SEQ ID NO: 731 - protein, 38 aa, synthetic construct
EYAASVKGRFTISRDDSKNSVYLQMNSLKTEDTAVYYC

SEQ ID NO: 732 - protein, 11 aa, synthetic construct
WGQGTLVTVSS

SEQ ID NO: 733 - protein, 26 aa, synthetic construct
  VARIANT 4: note = misc_feature - Xaa can be any naturally occurring amino acid
DIPXTQSPSFLSASVGDRVTITCRTS

SEQ ID NO: 734 - protein, 17 aa, synthetic construct
LAWYQQKPGKAPKLLIY

SEQ ID NO: 735 - protein, 36 aa, synthetic construct
TLQSGVPSRFSGSGSGTEFTLTISSLQPEDFATYYC

SEQ ID NO: 736 - protein, 10 aa, synthetic construct
FGQGTRLEIK

SEQ ID NO: 737 - protein, 448 aa, synthetic construct
  VARIANT 2..3: note = misc_feature - Xaa can be any naturally occurring amino acid
  VARIANT 145: note = misc_feature - Xaa can be any naturally occurring amino acid
  VARIANT 215: note = misc_feature - Xaa can be any naturally occurring amino acid
  VARIANT 359: note = misc_feature - Xaa can be any naturally occurring amino acid
CXXVESGGGLVQPGGSLRLSCAASGFTFSDYYMDWVRQAPGKGLEWVGRSRNKANSYTTEYAASVKGRFTISRDDSKNSVYLQMNSLKTEDTAVYYCARAFSGGSYCSDYWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 738 - protein, 211 aa, synthetic construct
  VARIANT 4: note = misc_feature - Xaa can be any naturally occurring amino acid
DIPXTQSPSFLSASVGDRVTITCRTSQGISSYLAWYQQKPGKAPKLLIYAASTLQSGVPSRFSGSGSGTEFTLTISSLQPEDFATYYCQQLNSYPSTFGQGTRLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 739 - DNA, 365 bp, synthetic construct
  misc_difference 6..9: note = misc_feature - n is a, c, g, or t
  misc_difference 13: note = misc_feature - n is a, c, g, or t
  source 1..365: mol_type = other DNA
gtgccnnnnggtngagtctgggggaggcttggtccagcctggagggtccctgagactctcctgtgcagcctctggattcaccttcagtgactactacatggactgggtccgccaggctccagggaaggggctggagtgggtaggccgttctagaaacaaagctaacagttacaccacagaatacgccgcgtctgtgaaaggcagattcaccatctcaagagatgattcaaagaactcagtatatctgcaaatgaacagcctgaaaaccgaggacacggccgtgtattactgtgcaagggctttctcgggtgggagctactgttccgactactggggccagggaaccctggtcaccgtctcctcag

SEQ ID NO: 740 - DNA, 322 bp, synthetic construct
  misc_difference 9..11: note = misc_feature - n is a, c, g, or t
  source 1..322: mol_type = other DNA
gacatcccnnngacccagtctccatccttcctgtctgcatctgtaggagacagagtcaccatcacttgccgtaccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacaacttaatagttacccgtccaccttcggccaagggacacgactggagattaaac

SEQ ID NO: 741 - protein, 8 aa, synthetic construct
  VARIANT 5: note = misc_feature - Xaa can be any naturally occurring amino acid
  VARIANT 7: note = misc_feature - Xaa can be any naturally occurring amino acid
GFTFXSXA

SEQ ID NO: 742 - protein, 8 aa, synthetic construct
  VARIANT 2..4: note = misc_feature - Xaa can be any naturally occurring amino acid
  VARIANT 7: note = misc_feature - Xaa can be any naturally occurring amino acid
IXXXGGXT

SEQ ID NO: 743 - skipped

SEQ ID NO: 744 - protein, 7 aa, synthetic construct
QPISSTY

SEQ ID NO: 745 - skipped

SEQ ID NO: 746 - protein, 10 aa, synthetic construct
QHYSNSPPYT

SEQ ID NO: 747 - protein, 107 aa, synthetic construct
  VARIANT 2: note = misc_feature - Xaa can be any naturally occurring amino acid
  VARIANT 5: note = misc_feature - Xaa can be any naturally occurring amino acid
  VARIANT 15: note = misc_feature - Xaa can be any naturally occurring amino acid
  VARIANT 17: note = misc_feature - Xaa can be any naturally occurring amino acid
  VARIANT 20: note = misc_feature - Xaa can be any naturally occurring amino acid
  VARIANT 25: note = misc_feature - Xaa can be any naturally occurring amino acid
  VARIANT 34..35: note = misc_feature - Xaa can be any naturally occurring amino acid
  VARIANT 37..39: note = misc_feature - Xaa can be any naturally occurring amino acid
  VARIANT 42: note = misc_feature - Xaa can be any naturally occurring amino acid
  VARIANT 44: note = misc_feature - Xaa can be any naturally occurring amino acid
  VARIANT 46..48: note = misc_feature - Xaa can be any naturally occurring amino acid
  VARIANT 54: note = misc_feature - Xaa can be any naturally occurring amino acid
  VARIANT 59: note = misc_feature - Xaa can be any naturally occurring amino acid
  VARIANT 62: note = misc_feature - Xaa can be any naturally occurring amino acid
  VARIANT 72..73: note = misc_feature - Xaa can be any naturally occurring amino acid
  VARIANT 78: note = misc_feature - Xaa can be any naturally occurring amino acid
  VARIANT 80: note = misc_feature - Xaa can be any naturally occurring amino acid
  VARIANT 82..88: note = misc_feature - Xaa can be any naturally occurring amino acid
  VARIANT 90..94: note = misc_feature - Xaa can be any naturally occurring amino acid
  VARIANT 96: note = misc_feature - Xaa can be any naturally occurring amino acid
  VARIANT 102: note = misc_feature - Xaa can be any naturally occurring amino acid
GXLRXSCAASGFTFXSXAMXWVRQXPGKGLEWVXXIXXXGGXTXYXXXVKGRFXISRDXSKXTLYLQMNSLXXEDTAXYXCXXXXXXXGXXXXXTXWGQGTXVTVSS

SEQ ID NO: 748 - protein, 109 aa, synthetic construct
  VARIANT 3..4: note = misc_feature - Xaa can be any naturally occurring amino acid
EIXXTQSPGTLSLSLGEGATLSCRASQPISSTYLTWYQQRPGQAPRLLIYATSTRATDIPDRFSGSGSGTDFTLTISRLETEDFAVYYCQHYSNSPPYTFGQGTKLEIK

SEQ ID NO: 749 - protein, 10 aa, synthetic construct
  VARIANT 2: note = misc_feature - Xaa can be any naturally occurring amino acid
  VARIANT 5: note = misc_feature - Xaa can be any naturally occurring amino acid
GXLRXSCAAS

SEQ ID NO: 750 - protein, 17 aa, synthetic construct
  VARIANT 2: note = misc_feature - Xaa can be any naturally occurring amino acid
  VARIANT 7: note = misc_feature - Xaa can be any naturally occurring amino acid
  VARIANT 16..17: note = misc_feature - Xaa can be any naturally occurring amino acid
MXWVRQXPGKGLEWVXX

SEQ ID NO: 751 - protein, 38 aa, synthetic construct
  VARIANT 1: note = misc_feature - Xaa can be any naturally occurring amino acid
  VARIANT 3..5: note = misc_feature - Xaa can be any naturally occurring amino acid
  VARIANT 11: note = misc_feature - Xaa can be any naturally occurring amino acid
  VARIANT 16: note = misc_feature - Xaa can be any naturally occurring amino acid
  VARIANT 19: note = misc_feature - Xaa can be any naturally occurring amino acid
  VARIANT 29..30: note = misc_feature - Xaa can be any naturally occurring amino acid
  VARIANT 35: note = misc_feature - Xaa can be any naturally occurring amino acid
  VARIANT 37: note = misc_feature - Xaa can be any naturally occurring amino acid
XYXXXVKGRFXISRDXSKXTLYLQMNSLXXEDTAXYXC

SEQ ID NO: 752 - protein, 11 aa, synthetic construct
  VARIANT 6: note = misc_feature - Xaa can be any naturally occurring amino acid
WGQGTXVTVSS

SEQ ID NO: 753 - protein, 26 aa, synthetic construct
  VARIANT 3..4: note = misc_feature - Xaa can be any naturally occurring amino acid
EIXXTQSPGTLSLSLGEGATLSCRAS

SEQ ID NO: 754 - protein, 17 aa, synthetic construct
LTWYQQRPGQAPRLLIY

SEQ ID NO: 755 - protein, 36 aa, synthetic construct
TRATDIPDRFSGSGSGTDFTLTISRLETEDFAVYYC

SEQ ID NO: 756 - protein, 10 aa, synthetic construct
FGQGTKLEIK

SEQ ID NO: 757 - protein, 434 aa, synthetic construct
  VARIANT 2: note = misc_feature - Xaa can be any naturally occurring amino acid
  VARIANT 5: note = misc_feature - Xaa can be any naturally occurring amino acid
  VARIANT 15: note = misc_feature - Xaa can be any naturally occurring amino acid
  VARIANT 17: note = misc_feature - Xaa can be any naturally occurring amino acid
  VARIANT 20: note = misc_feature - Xaa can be any naturally occurring amino acid
  VARIANT 25: note = misc_feature - Xaa can be any naturally occurring amino acid
  VARIANT 34..35: note = misc_feature - Xaa can be any naturally occurring amino acid
  VARIANT 37..39: note = misc_feature - Xaa can be any naturally occurring amino acid
  VARIANT 42: note = misc_feature - Xaa can be any naturally occurring amino acid
  VARIANT 44: note = misc_feature - Xaa can be any naturally occurring amino acid
  VARIANT 46..48: note = misc_feature - Xaa can be any naturally occurring amino acid
  VARIANT 54: note = misc_feature - Xaa can be any naturally occurring amino acid
  VARIANT 59: note = misc_feature - Xaa can be any naturally occurring amino acid
  VARIANT 62: note = misc_feature - Xaa can be any naturally occurring amino acid
  VARIANT 72..73: note = misc_feature - Xaa can be any naturally occurring amino acid
  VARIANT 78: note = misc_feature - Xaa can be any naturally occurring amino acid
  VARIANT 80: note = misc_feature - Xaa can be any naturally occurring amino acid
  VARIANT 82..88: note = misc_feature - Xaa can be any naturally occurring amino acid
  VARIANT 90..94: note = misc_feature - Xaa can be any naturally occurring amino acid
  VARIANT 96: note = misc_feature - Xaa can be any naturally occurring amino acid
  VARIANT 102: note = misc_feature - Xaa can be any naturally occurring amino acid
  VARIANT 131: note = misc_feature - Xaa can be any naturally occurring amino acid
  VARIANT 201: note = misc_feature - Xaa can be any naturally occurring amino acid
  VARIANT 345: note = misc_feature - Xaa can be any naturally occurring amino acid
GXLRXSCAASGFTFXSXAMXWVRQXPGKGLEWVXXIXXXGGXTXYXXXVKGRFXISRDXSKXTLYLQMNSLXXEDTAXYXCXXXXXXXGXXXXXTXWGQGTXVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 758 - protein, 213 aa, synthetic construct
  VARIANT 3..4: note = misc_feature - Xaa can be any naturally occurring amino acid
EIXXTQSPGTLSLSLGEGATLSCRASQPISSTYLTWYQQRPGQAPRLLIYATSTRATDIPDRFSGSGSGTDFTLTISRLETEDFAVYYCQHYSNSPPYTFGQGTKLEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 759 - DNA, 322 bp, synthetic construct
  misc_difference 5: note = misc_feature - n is a, c, g, or t
  misc_difference 13: note = misc_feature - n is a, c, g, or t
  misc_difference 43..44: note = misc_feature - n is a, c, g, or t
  misc_difference 114: note = misc_feature - n is a, c, g, or t
  misc_difference 248: note = misc_feature - n is a, c, g, or t
  misc_difference 256: note = misc_feature - n is a, c, g, or t
  misc_difference 268: note = misc_feature - n is a, c, g, or t
  misc_difference 280..281: note = misc_feature - n is a, c, g, or t
  misc_difference 288: note = misc_feature - n is a, c, g, or t
  misc_difference 322: note = misc_feature - n is a, c, g, or t
  source 1..322: mol_type = other DNA
ggggncctgagantctcctgtgcagcctctggattcacctttnncagywakgccatgasctgggtccgccagrctccagggaagggrctggagtgggtyksmsstattarwrgnrrtggtggkasmacakactacrcwgmmyccgtgaarggcmgrttcaycatytcmagagayrattcmaarracacgytgtatctrcaratgaayagyctgararccgaggacacrgccstmtattwctgtrcsanaakaysanaykgwrrtggsnctyaytrctwynngactwcntggggccagggaacccwggtcaccgtctcctcan

SEQ ID NO: 760 - DNA, 328 bp, synthetic construct
  misc_difference 8..10: note = misc_feature - n is a, c, g, or t
  source 1..328: mol_type = other DNA
gaaattgnnntgacgcagtctccaggcaccctgtctttgtctctaggtgaaggagccaccctctcctgcagggccagtcagcctattagtagcacctacttaacctggtaccagcagagacctggccaggctccccggctcctcatctacgctacgtccaccagggccactgacatcccagacaggttcagtggcagtggctctgggacagacttcactctcaccatcagtagactggagactgaagattttgcagtgtactactgtcaacattattctaactcacctccgtacacttttggccaggggaccaagctggagatcaaac

SEQ ID NO: 761 - protein, 8 aa, synthetic construct
GFPFDSHA

SEQ ID NO: 762 - protein, 8 aa, synthetic construct
ISNNGASA

SEQ ID NO: 763 - protein, 17 aa, synthetic construct
ARTQYYDSRGFYFSLDA

SEQ ID NO: 764 - protein, 7 aa, synthetic construct
QPISSTY

SEQ ID NO: 765 - skipped

SEQ ID NO: 766 - protein, 10 aa, synthetic construct
QHYSNSPPYT

SEQ ID NO: 767 - protein, 119 aa, synthetic construct
ESGGGVVQPGRSVRLSCVGSGFPFDSHAIHWVRQAPGWGLEWVAVISNNGASAHYTDSVKGRFTVSRDNSKHTVYLLLNSLTKEDTAVYYCARTQYYDSRGFYFSLDAWGQGTLVTVSS

SEQ ID NO: 768 - protein, 109 aa, synthetic construct; identical to SEQ ID NO: 748
  VARIANT 3..4: note = misc_feature - Xaa can be any naturally occurring amino acid

SEQ ID NO: 769 - protein, 20 aa, synthetic construct
ESGGGVVQPGRSVRLSCVGS

SEQ ID NO: 770 - protein, 17 aa, synthetic construct
IHWVRQAPGWGLEWVAV

SEQ ID NO: 771 - protein, 38 aa, synthetic construct
HYTDSVKGRFTVSRDNSKHTVYLLLNSLTKEDTAVYYC

SEQ ID NO: 772 - protein, 11 aa, synthetic construct
WGQGTLVTVSS

SEQ ID NO: 773 - protein, 26 aa, synthetic construct
  VARIANT 3..4: note = misc_feature - Xaa can be any naturally occurring amino acid
EIXXTQSPGTLSLSLGEGATLSCRAS

SEQ ID NO: 774 - protein, 17 aa, synthetic construct
LTWYQQRPGQAPRLLIY

SEQ ID NO: 775 - protein, 36 aa, synthetic construct; identical to SEQ ID NO: 755

SEQ ID NO: 776 - protein, 10 aa, synthetic construct
FGQGTKLEIK

SEQ ID NO: 777 - protein, 446 aa, synthetic construct
  VARIANT 143: note = misc_feature - Xaa can be any naturally occurring amino acid
  VARIANT 213: note = misc_feature - Xaa can be any naturally occurring amino acid
  VARIANT 357: note = misc_feature - Xaa can be any naturally occurring amino acid
ESGGGVVQPGRSVRLSCVGSGFPFDSHAIHWVRQAPGWGLEWVAVISNNGASAHYTDSVKGRFTVSRDNSKHTVYLLLNSLTKEDTAVYYCARTQYYDSRGFYFSLDAWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 778 - protein, 213 aa, synthetic construct; identical to SEQ ID NO: 758
  VARIANT 3..4: note = misc_feature - Xaa can be any naturally occurring amino acid

SEQ ID NO: 779 - DNA, 358 bp, synthetic construct
  source 1..358: mol_type = other DNA
gagtctgggggaggcgttgtccagcctgggaggtccgtgagactctcctgtgtaggctctggattcccattcgatagtcatgccatacactgggtccgccaggctccaggctggggcctggagtgggtggcagttatctcaaacaatggcgccagtgcacattacacagactccgtgaagggccgattcaccgtctccagagacaattccaagcacaccgtctatctacttctgaacagtctgacaaaagaagacacggctgtctactactgtgcgaggacacaatattatgatagtcgtggtttttacttttctctcgacgcctggggccagggaaccctcgtcaccgtctcctcag

SEQ ID NO: 780 - DNA, 328 bp, synthetic construct; identical to SEQ ID NO: 760
  misc_difference 8..10: note = misc_feature - n is a, c, g, or t
  source 1..328: mol_type = other DNA

SEQ ID NO: 781 - protein, 8 aa, synthetic construct
GFTFSKAW

SEQ ID NO: 782 - protein, 10 aa, synthetic construct
IKSKIDDGTV

SEQ ID NO: 783 - protein, 15 aa, synthetic construct
TTVPYCNGTHCYGTA

SEQ ID NO: 784 - protein, 12 aa, synthetic construct
QSLLYSGNNKNY

SEQ ID NO: 785 - skipped

SEQ ID NO: 786 - protein, 6 aa, synthetic construct
QRHWET

SEQ ID NO: 787 - protein, 124 aa, synthetic construct
  VARIANT 3..5: note = misc_feature - Xaa can be any naturally occurring amino acid
VPXXXESGGALVKPGGSLRLSCEASGFTFSKAWMSWVRQAPGKGLEWVGRIKSKIDDGTVDYDAPVKGRFTIARDDAKNTLYLQMNSLRTDDTAVYYCTTVPYCNGTHCYGTAWGQGTLVTVSS

SEQ ID NO: 788 - protein, 110 aa, synthetic construct
DIVMTQSPDSLAVSLGERATINCKSSQSLLYSGNNKNYLTWYQQKPGQPPKLLIYWASIRESGVPDRFSGGGSGTDFTLTISSLQAEDVAIYYCQRHWETFGQGTRVEIK

SEQ ID NO: 789 - protein, 25 aa, synthetic construct
  VARIANT 3..5: note = misc_feature - Xaa can be any naturally occurring amino acid
VPXXXESGGALVKPGGSLRLSCEAS

SEQ ID NO: 790 - protein, 17 aa, synthetic construct
MSWVRQAPGKGLEWVGR

SEQ ID NO: 791 - protein, 38 aa, synthetic construct
DYDAPVKGRFTIARDDAKNTLYLQMNSLRTDDTAVYYC

SEQ ID NO: 792 - protein, 11 aa, synthetic construct
WGQGTLVTVSS

SEQ ID NO: 793 - protein, 26 aa, synthetic construct
DIVMTQSPDSLAVSLGERATINCKSS

SEQ ID NO: 794 - protein, 17 aa, synthetic construct
LTWYQQKPGQPPKLLIY

SEQ ID NO: 795 - protein, 36 aa, synthetic construct
IRESGVPDRFSGGGSGTDFTLTISSLQAEDVAIYYC

SEQ ID NO: 796 - protein, 10 aa, synthetic construct
FGQGTRVEIK

SEQ ID NO: 797 - protein, 451 aa, synthetic construct
  VARIANT 3..5: note = misc_feature - Xaa can be any naturally occurring amino acid
  VARIANT 148: note = misc_feature - Xaa can be any naturally occurring amino acid
  VARIANT 218: note = misc_feature - Xaa can be any naturally occurring amino acid
  VARIANT 362: note = misc_feature - Xaa can be any naturally occurring amino acid
VPXXXESGGALVKPGGSLRLSCEASGFTFSKAWMSWVRQAPGKGLEWVGRIKSKIDDGTVDYDAPVKGRFTIARDDAKNTLYLQMNSLRTDDTAVYYCTTVPYCNGTHCYGTAWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 798 - protein, 214 aa, synthetic construct
DIVMTQSPDSLAVSLGERATINCKSSQSLLYSGNNKNYLTWYQQKPGQPPKLLIYWASIRESGVPDRFSGGGSGTDFTLTISSLQAEDVAIYYCQRHWETFGQGTRVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 799 - DNA, 373 bp, synthetic construct
  misc_difference 7..15: note = misc_feature - n is a, c, g, or t
  source 1..373: mol_type = other DNA
gtgccannnnnnnnngagtctgggggagccttggtaaagccgggggggtcccttagactctcctgtgaagcctctggattcactttcagtaaggcctggatgagttgggtccgtcaggccccagggaagggtctggaatgggtcggccgtattaaaagtaagattgatgatgggactgtcgactacgatgcacccgtgaaaggcagattcaccatcgcaagagatgatgccaaaaatacactctatctgcaaatgaatagcctgagaaccgacgacacagccgtgtattactgtaccacagttccctattgtaatgggactcactgttatgggactgcgtggggccagggaaccctggtcaccgtctcctcag

SEQ ID NO: 800 - DNA, 331 bp, synthetic construct
  source 1..331: mol_type = other DNA
gacatcgtgatgacccagtctccagactccctggctgtgtctctgggcgagagggccaccatcaactgcaagtccagccagagtcttttgtacagcggcaacaataagaactacttaacttggtaccagcagaaaccaggacagcctcctaagttgctcatttactgggcatctatccgggagtccggggtccctgaccgattcagtggcggcgggtctgggacagatttcactctcaccatcagcagcctgcaggctgaagatgtggctatttattactgtcagcgacattgggagacgttcggccaagggaccagggttgagatcaaac

SEQ ID NO: 801 - protein, 8 aa, synthetic construct
GFTFSTFA

SEQ ID NO: 802 - protein, 8 aa, synthetic construct
ISDNGNRK

SEQ ID NO: 803 - protein, 17 aa, synthetic construct
AKTRDYDSRGYYFGLDH

SEQ ID NO: 804 - protein, 7 aa, synthetic construct
QSVISNY

SEQ ID NO: 805 - skipped

SEQ ID NO: 806 - protein, 10 aa, synthetic construct
QHYGNSPPYT

SEQ ID NO: 807 - protein, 123 aa, synthetic construct
  VARIANT 2: note = misc_feature - Xaa can be any naturally occurring amino acid
  VARIANT 91: note = misc_feature - Xaa can be any naturally occurring amino acid
CXLVESGGGVVQPGRSLRLSCAASGFTFSTFALHWVRQAPGKGLEWMAVISDNGNRKDYADSVKGRFTISRDNSENTLYLEMNSLRPEDTXVYYCAKTRDYDSRGYYFGLDHWGQGTLVTVSS

SEQ ID NO: 808 - protein, 109 aa, synthetic construct
  VARIANT 4: note = misc_feature - Xaa can be any naturally occurring amino acid
EIGXTQSPGTLSLSPGERATLSCRASQSVISNYLAWYQHKPGQAPRLLIYAASSRATDIPDRFSGSGSGTDFILTISRLEPEDFAVYYCQHYGNSPPYTFGQGTKVEIK

SEQ ID NO: 809 - protein, 24 aa, synthetic construct
  VARIANT 2: note = misc_feature - Xaa can be any naturally occurring amino acid
CXLVESGGGVVQPGRSLRLSCAAS

SEQ ID NO: 810 - protein, 17 aa, synthetic construct
LHWVRQAPGKGLEWMAV

SEQ ID NO: 811 - protein, 38 aa, synthetic construct
  VARIANT 34: note = misc_feature - Xaa can be any naturally occurring amino acid
DYADSVKGRFTISRDNSENTLYLEMNSLRPEDTXVYYC

SEQ ID NO: 812 - protein, 11 aa, synthetic construct
WGQGTLVTVSS

SEQ ID NO: 813 - protein, 26 aa, synthetic construct
  VARIANT 4: note = misc_feature - Xaa can be any naturally occurring amino acid
EIGXTQSPGTLSLSPGERATLSCRAS

SEQ ID NO: 814 - protein, 17 aa, synthetic construct
LAWYQHKPGQAPRLLIY

SEQ ID NO: 815 - protein, 36 aa, synthetic construct
SRATDIPDRFSGSGSGTDFILTISRLEPEDFAVYYC

SEQ ID NO: 816 - protein, 10 aa, synthetic construct
FGQGTKVEIK

SEQ ID NO: 817 - protein, 450 aa, synthetic construct
  VARIANT 2: note = misc_feature - Xaa can be any naturally occurring amino acid
  VARIANT 91: note = misc_feature - Xaa can be any naturally occurring amino acid
  VARIANT 147: note = misc_feature - Xaa can be any naturally occurring amino acid
  VARIANT 217: note = misc_feature - Xaa can be any naturally occurring amino acid
  VARIANT 361: note = misc_feature - Xaa can be any naturally occurring amino acid
CXLVESGGGVVQPGRSLRLSCAASGFTFSTFALHWVRQAPGKGLEWMAVISDNGNRKDYADSVKGRFTISRDNSENTLYLEMNSLRPEDTXVYYCAKTRDYDSRGYYFGLDHWGQGTLVTVSSKGPSVFPLAPSSKSTSGGTAALGXLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 818 - protein, 213 aa, synthetic construct
  VARIANT 4: note = misc_feature - Xaa can be any naturally occurring amino acid
EIGXTQSPGTLSLSPGERATLSCRASQSVISNYLAWYQHKPGQAPRLLIYAASSRATDIPDRFSGSGSGTDFILTISRLEPEDFAVYYCQHYGNSPPYTFGQGTKVEIKAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECK

SEQ ID NO: 819 - DNA, 372 bp, synthetic construct
  misc_difference 7..8: note = misc_feature - n is a, c, g, or t
  source 1..372: mol_type = other DNA
ggtgccnnctggtggagtctgggggaggcgtggtccagcctgggaggtccctgagactctcctgtgcagcctctggattcacatttagtacctttgctctacattgggtccgccaggctccaggcaaggggctggagtggatggccgttatatcagataatgggaatagaaaagactacgcagactccgtgaagggccgattcaccatctcccgagacaactccgagaacacactgtatctggaaatgaacagcctgagacctgaggacacggmtgtctattactgtgcgaaaacaagggactatgatagccgtggttattacttcggtcttgaccactggggccagggaaccctggtcaccgtctcctcag

SEQ ID NO: 820 - DNA, 328 bp, synthetic construct
  misc_difference 9..12: note = misc_feature - n is a, c, g, or t
  source 1..328: mol_type = other DNA
gaaataggnnnnacgcagtctccaggcaccctgtctttgtctccaggggaaagagccaccctctcctgcagggccagtcagagtgttatcagcaactacttagcctggtatcagcacaaacctggccaggctcccaggctcctcatctatgctgcctccagcagggccactgacatcccagacaggttcagtggcagtgggtctgggacagacttcattctcaccatcagcagactggagcctgaagattttgcggtatactactgtcagcactatggtaactcacctccgtacacttttggccagggaaccaaggtggagattaaac

SEQ ID NO: 821 - protein, 172 aa, Arachis hypogaea
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 822 - protein, 11 aa, synthetic construct
  VARIANT 1: note = MISC_FEATURE - Xaa is Val or Ala
XKDNGWRAFDH

SEQ ID NO: 823 - protein, 9 aa, synthetic construct
  VARIANT 3: note = MISC_FEATURE - Xaa is Ser or Ala
  VARIANT 6: note = MISC_FEATURE - Xaa is Asn or Thr
MQXLEXPWT

SEQ ID NO: 824 - protein, 8 aa, synthetic construct
  VARIANT 2: note = MISC_FEATURE - Xaa is Asp or Gly
  VARIANT 4: note = MISC_FEATURE - Xaa is Ile or Met
GXSXSSYY

SEQ ID NO: 825 - protein, 7 aa, synthetic construct
  VARIANT 4: note = MISC_FEATURE - Xaa is Ser or Thr
IFTXGST

SEQ ID NO: 826 - protein, 19 aa, synthetic construct
  VARIANT 1: note = MISC_FEATURE - Xaa is Ala or Val
  VARIANT 6: note = MISC_FEATURE - Xaa is Ala or Gly
  VARIANT 7: note = MISC_FEATURE - Xaa is Leu or Arg
  VARIANT 9: note = MISC_FEATURE - Xaa is Ser or His
  VARIANT 11: note = MISC_FEATURE - Xaa is Gly or Ser
XRDRRXXSXDXNWYWYFDL

SEQ ID NO: 827 - protein, 6 aa, synthetic construct
  VARIANT 3: note = MISC_FEATURE - Xaa is Ile or Leu
  VARIANT 4: note = MISC_FEATURE - Xaa is Thr or Ser
QTXXRN

(19) United States
(12) Patent Application Publication
(10) Pub. No.: US 2026/0317407 A1
(43) Pub. Date: Oct. 15, 2026

(54) THERAPEUTIC NEUTRALIZATION ANTIBODIES FOR THE TREATMENT OF PEANUT ALLERGY

(71) Applicants: The General Hospital Corporation, Boston, MA (US); Massachusetts Institute of Technology, Cambridge, MA (US)

(72) Inventors: Sarita U. PATIL, Newton, MA (US); Wayne G. SHREFFLER, Beverly, MA (US); John Christopher LOVE, Somerville, MA (US)

(21) Appl. No.: 19/321,494

(22) Filed: Sep. 8, 2025

Related U.S. Application Data
(63) Continuation of application No. 17/762,902, filed on Sep. 25, 2020, now Pat. No. 12,435,127.
(60) Provisional application No. 62/905,698, filed on Sep. 25, 2019.

(51) Int. Cl. C07K 16/16; A61P 37/00; G01N 33/68

(52) U.S. Cl. CPC C07K 16/16; A61P 37/00; G01N 33/6854; C07K 2317/21; C07K 2317/30; C07K 2317/92

(57) ABSTRACT

The invention provides anti-Ara h 2 antibodies (e.g., an anti-Ara h 2 neutralizing antibody) and methods of using the same, e.g., for treating and/or preventing peanut allergy or sensitivity. Also provided herein are anti-Ara h 2 antibodies and methods of using the same, e.g., for diagnostics and methods of monitoring peanut oral immunotherapy.

### CROSS-REFERENCE TO RELATED APPLICATIONS

This application claims benefit of U.S. Provisional Application No. 62/905,698, filed Sep. 25, 2019, the contents of which are incorporated herein by reference in their entirety.

### STATEMENT AS TO FEDERALLY FUNDED RESEARCH

This invention was made with government support under AI121491 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 10, 2025, is named “51343-003003_Sequence_Listing_9_10_25” and is 933,045 bytes in size.

### FIELD OF THE INVENTION

The present invention is related to human antibodies and antigen-binding fragments of human antibodies that specifically bind to a peanut allergen, therapeutic compositions comprising the antibodies and methods of using those antibodies including diagnostic uses.

### BACKGROUND OF THE INVENTION

Peanut allergy is one of the most common causes of severe food allergy attacks. Peanut allergy symptoms can be life-threatening (anaphylaxis). For some people with peanut allergy, even miniscule amounts of peanuts can cause a serious reaction.

Rates of food allergy have nearly tripled in the past two decades, now affecting about 8% of children in the US, and incurring $25 billion in costs annually. There are over 3 million people in the US. More than 1% of children in the United States and Europe have peanut allergy caused by IgE, or allergy antibodies. They can have severe, even life-threatening allergic responses, known as anaphylaxis, to even tiny exposures to peanut. Peanut allergy tends to be persistent even after childhood in about 80% of affected patients. Currently, the only available treatment is avoidance and emergent use of epinephrine, which places our most vulnerable population at significant risk of mortality and morbidity. Recently, Palforzia, an oral immunotherapy (OIT) has been approved by the FDA as a treatment for food allergy.

OIT clinical trials conducted at Massachusetts General Hospital, like others, have consistently shown that less than one-third of patients develop lasting tolerance after cessation of peanut OIT, and the rate of reactions and anaphylaxis requiring epinephrine during OIT poses a serious and significant burden. Therefore, even with potential future implementation of OIT, a significant portion of patients with IgE-mediated food allergy will require treatment to prevent anaphylactic reactions on accidental encounters with peanut allergen.

A need therefore exists for compositions and methods for treating peanut allergy.

### SUMMARY OF THE INVENTION

The invention provides antibodies and antigen-binding fragments thereof that bind specifically to the peanut allergen, Ara h 2. Such antibodies may be useful to bind the Ara h 2 allergen in vivo following exposure of a sensitized patient to a peanut allergen, and as such, may act to either promote clearance of Ara h 2, neutralization of the effects of Ara h 2 or to block the binding of the allergen to pre-formed IgE on the surface of mast cells or basophils. By doing so, the antibodies disclosed herein may prevent the release of histamine or other inflammatory mediators from mast cells or basophils, thereby preventing or diminishing the untoward effects observed in patients sensitized to a peanut allergen such as Ara h 2.

Peanut allergy occurs when an individual's immune system mistakenly identifies peanut proteins such as Ara h 2 as something harmful. Direct or indirect contact with peanuts causes the individual's immune system to release symptom-causing chemicals such as histamines into their bloodstream. Exposure to peanuts can trigger an allergic reaction and can occur in various ways including, without limitation, direct contact (e.g., eating peanuts or peanut-containing foods or through direct skin contact with peanuts); cross-contact (e.g., an unintended introduction of peanuts into a product) or inhalation (e.g., an allergic reaction may occur if an individual inhales dust or aerosols containing peanuts, from a source such as peanut flour or peanut oil cooking spray). In certain applications, the antibodies described herein may reduce, minimize, or prevent at least one symptom in a patient sensitive to the Ara h 2 peanut allergen, such as skin reactions such as hives, redness or swelling, itching or tingling in or around the mouth or throat, digestive problems such as diarrhea, stomach cramps, nausea, or vomiting, tightening of the throat, sneezing, congestion, nasal blockage, coughing, wheezing, bronchoconstriction, rhinitis, or shortness of breath. In other applications, the antibodies may be capable of preventing even more serious in vivo complications associated with exposure to the peanut allergen in sensitized individuals, such as asthmatic responses, swelling of the throat that makes it difficult to breath, a severe drop in blood pressure (shock), rapid pulse, dizziness, lightheadedness or loss of consciousness, anaphylaxis, or even death. Children and adults who have a severe peanut allergy are especially at risk of having this life-threatening reaction.

The anti-Ara h 2 antibody compositions and methods disclosed herein address an unmet need in the art.

Expert guidelines for treatment of peanut allergy recommend that, for example, patients are counseled to avoid the allergen and to use injectable epinephrine for treatment of anaphylaxis should exposure occur. However, up to 25% of pediatric visits to an emergency department are for treatment of IgE-mediated hypersensitivity, and there is growing recognition of the burden of the costs of this strategy, both in direct medical costs as well as indirect costs. The financial, social, and psychological burden of food allergy is considerable, and widely evident in our daily lives.

Passive protection using peanut-specific protective antibodies disclosed herein provide an extra margin of safety for patients with IgE-mediated food allergies, for example, by preventing allergic reactions to accidental ingestion or exposure. One of the most common reasons that patients and their families seek further therapy for food allergy is for the prevention of IgE-mediated food allergic reactions. Passive protection allows clinicians to provide a therapy with low risk for allergic reactions and which can provide long-lasting protection against reactions to accidental exposures.

Furthermore, patients who are not eligible for other treatments such as OIT, due to underlying eosinophilic esophagitis or extreme sensitivity to the allergen, would still be able to tolerate a strategy of passive protection. In clinical trials, the majority of patients experience side effects and even those who undergo OIT can still have episodes of treatment-related anaphylaxis. Passive protection offers the possibility of treatment without the side effects of allergen exposure. Lastly, passive immunization with peanut-specific antibodies may be an effective adjunctive therapy to immunotherapy as a strategy to decrease the high rate of adverse events known to occur with immunotherapy.

The development of peanut-specific antibodies that effectively neutralize the allergen further provide a useful diagnostic tool for monitoring clinical tolerance development during allergen-specific immunotherapy. In most forms of immunotherapy, both oral and sublingual, patients are subjected to repeated oral challenges to evaluate the development of tolerance. Suppression of effector cells and basophils has been shown to be effective biomarkers of tolerance in OIT, and that this suppression is mediated by antibodies in the serum.

Furthermore, a competitive assay using the antibodies described herein would be highly effective for monitoring tolerance during immunotherapy. In this assay, a comparison of individuals' OIT-induced allergen-specific antibodies with the pre-defined protective antibodies is utilized to determine whether their post-OIT antibody repertoire will effectively protect them against peanut exposures, without the need for repeated financially and medically intensive oral food challenges. As OIT is increasingly adopted outside of clinical trials, a robust methodology of monitoring tolerance is needed. The diagnostic test disclosed herein that relies on patient serum is superior as opposed to the methodology employed in previous clinical trials, which relies of a technically challenging and biologically variable assay that activates short-lived effector cells.

In one aspect, the invention features an isolated antibody that binds to Ara h 2 (conglutin-7), or an antigen-binding fragment thereof, wherein the antibody comprises the following six complementarity determining regions (CDRs): (a) an CDR-H1 comprising the amino acid sequence of GFTFRDYS (SEQ ID NO: 1); (b) an CDR-H2 comprising the amino acid sequence of IRFDGTTK (SEQ ID NO: 2); (c) an CDR-H3 comprising the amino acid sequence of X1KDNGWRAFDH (SEQ ID NO: 822), wherein X1 is V or A; (d) an CDR-L1 comprising the amino acid sequence of QSLLHRNGYIY (SEQ ID NO: 4); (e) an CDR-L2 comprising the amino acid sequence of FVS (SEQ ID NO: 5); and (f) an CDR-L3 comprising the amino acid sequence of MQX1LEX2PWT (SEQ ID NO: 823), wherein X1 is S or A and X2 is N or T.

In another aspect, the invention features an isolated antibody that binds to Ara h 2 (conglutin-7), or an antigen-binding fragment thereof, wherein the antibody comprises the following six CDRs: (a) an CDR-H1 comprising the amino acid sequence of GFTFRDYS (SEQ ID NO: 1); (b) an CDR-H2 comprising the amino acid sequence of IRFDGTTK (SEQ ID NO: 2); (c) an CDR-H3 comprising the amino acid sequence of VKDNGWRAFDH (SEQ ID NO: 3); (d) an CDR-L1 comprising the amino acid sequence of QSLLHRNGYIY (SEQ ID NO: 4); (e) an CDR-L2 comprising the amino acid sequence of FVS (SEQ ID NO: 5); and (f) an CDR-L3 comprising the amino acid sequence of MQSLENPWT (SEQ ID NO: 6).

In some embodiments of either of the previous aspects, the antibody comprises (a) a heavy chain variable (VH) domain comprising an amino sequence having at least 90%, at least 95%, or at least 99% sequence identity to the amino acid sequence of SEQ ID NO: 7; (b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b). In some embodiments, the antibody further comprises the following VH domain framework regions (FRs): (a) an FR-H1 comprising the amino acid sequence of VESGGGVVQPGASLRLSCVAS (SEQ ID NO: 9); (b) an FR-H2 comprising the amino acid sequence of MHWVRQAPGKGLEWMSF (SEQ ID NO: 10); (c) an FR-H3 comprising the amino acid sequence of DYRESVKGRFIISRDDSKNTLYLQMTSLRPEDTARYYC (SEQ ID NO: 11); and (d) an FR-H4 comprising the amino acid sequence of WGQGALVIVSS (SEQ ID NO: 12). In some embodiments, the VH domain comprises the amino acid sequence of SEQ ID NO: 7.

In some embodiments, the antibody further comprising the following VL domain FRs: (a) an FR-L1 comprising the amino acid sequence of EIVMTQSPLSLPVTPGEAASISCRSS (SEQ ID NO: 13); (b) an FR-L2 comprising the amino acid sequence of LDWYLQRPGQSPQLLIS (SEQ ID NO: 14); (c) an FR-L3 comprising the amino acid sequence of KRASGAPDRFSGSGSGTDFTLTISRVEAEDFGVYFC (SEQ ID NO: 15); and (d) an FR-L4 comprising the amino acid sequence of FGP (SEQ ID NO: 16). In some embodiments, the VL domain comprises the amino acid sequence of SEQ ID NO: 8.

In another aspect, the invention features an isolated antibody that binds to Ara h 2, or an antigen-binding fragment thereof, wherein the antibody comprises (a) a VH domain comprising an amino acid sequence having at least 90%, at least 95%, or at least 99% sequence identity to the amino acid sequence of SEQ ID NO: 7 and (b) a VL domain comprising an amino acid sequence having at least 90%, at least 95%, or at least 99% sequence identity to the amino acid sequence of SEQ ID NO: 8.

In some embodiments, the antibody comprises a VH domain comprising the amino acid sequence of SEQ ID NO: 7 and a VL domain comprising the amino acid sequence of SEQ ID NO: 8.

In some embodiments of any of the previous aspects, the antibody comprises (a) a heavy chain comprising the amino acid sequence of SEQ ID NO: 17 and (b) a light chain comprising the amino acid sequence of SEQ ID NO: 18.

In some embodiments of any of the previous aspects, the antibody binds Ara h 2 with a K_{D} of about 50 nM or less. In some embodiments, the K_{D} is measured by a surface plasmon resonance assay. In some embodiments, the antibody binds Ara h 2 with a K_{D} of between about 0.1 pM and about 40 nM. In some embodiments, the antibody binds Ara h 2 with a K_{D} of between about 120 pM and about 20 nM. In some embodiments, the antibody binds Ara h 2 with a K_{D} of between about 120 pM and about 10 nM.

In another aspect, the invention features an isolated antibody that binds to Ara h 2 (conglutin-7), or an antigen-binding fragment thereof, wherein the antibody comprises the following six CDRs: (a) an CDR-H1 comprising the amino acid sequence of GFTFRDYS (SEQ ID NO: 21); (b) an CDR-H2 comprising the amino acid sequence of IRFDGTTK (SEQ ID NO: 22); (c) an CDR-H3 comprising the amino acid sequence of AKDNGWRAFDH (SEQ ID NO: 23); (d) an CDR-L1 comprising the amino acid sequence of QSLLHRNGYIY (SEQ ID NO: 24); (e) an CDR-L2 comprising the amino acid sequence of FVS (SEQ ID NO: 25); and (f) an CDR-L3 comprising the amino acid sequence of MQALETPWT (SEQ ID NO: 26). In some embodiments, the antibody comprises (a) a VH domain comprising an amino sequence having at least 90%, at least 95%, or at least 99% sequence identity to the amino acid sequence of SEQ ID NO: 27; (b) a VL domain comprising an amino acid sequence having at least 90%, at least 95%, or at least 99% identity to the amino acid sequence of SEQ ID NO: 28; or (c) a VH domain as in (a) and a VL domain as in (b). In some embodiments, the antibody further comprises the following VH domain FRs: (a) an FR-H1 comprising the amino acid sequence of GGGVVQPGTSLRLSCVAS (SEQ ID NO: 29); (b) an FR-H2 comprising the amino acid sequence of MHWVRQAPGKGLEWVSF (SEQ ID NO: 30); (c) an FR-H3 comprising the amino acid sequence of DYRDSVKGRFIISRDDSKNTLYLQMTSLRVEDTALYYC (SEQ ID NO: 31); and (d) an FR-H4 comprising the amino acid sequence of WGQGALVTVSS (SEQ ID NO: 32). In some embodiments, the VH domain comprises the amino acid sequence of SEQ ID NO: 27. In some embodiments, the antibody further comprises the following VL domain FRs: (a) an FR-L1 comprising the amino acid sequence of VMTQSPLSLPVTPGEAASISCRSS (SEQ ID NO: 33); (b) an FR-L2 comprising the amino acid sequence of LDWYLQRPGQSPQLLIS (SEQ ID NO: 34); (c) an FR-L3 comprising the amino acid sequence of KRASGAPDRFSGSGSGTDFTLTISRVEAEDFGVYFC (SEQ ID NO: 35); and (d) an FR-L4 comprising the amino acid sequence of FGPGTKLEIK (SEQ ID NO: 36). In some embodiments, the VL domain comprises the amino acid sequence of SEQ ID NO: 28.

In another aspect, the invention features an isolated antibody that binds to Ara h 2, or an antigen-binding fragment thereof, wherein the antibody comprises: (a) a VH domain comprising an amino acid sequence having at least 90%, at least 95%, or at least 99% sequence identity to the amino acid sequence of SEQ ID NO: 27 and (b) a VL domain comprising an amino acid sequence having at least 90%, at least 95%, or at least 99% sequence identity to the amino acid sequence of SEQ ID NO: 28. In some embodiments, the antibody comprises a VH domain comprising the amino acid sequence of SEQ ID NO: 27 and a VL domain comprising the amino acid sequence of SEQ ID NO: 28. In some embodiments, the antibody comprises (a) a heavy chain comprising the amino acid sequence of SEQ ID NO: 37 and (b) a light chain comprising the amino acid sequence of SEQ ID NO: 38.

In some embodiments of any of the previous aspects, the antibody binds Ara h 2 with a K_{D} of about 50 nM or less. In some embodiments, the K_{D} is measured by a surface plasmon resonance assay. In some embodiments, the antibody binds Ara h 2 with a K_{D} of between about 0.1 pM and about 40 nM. In some embodiments, the antibody binds Ara h 2 with a K_{D} of between about 120 pM and about 20 nM. In some embodiments, the antibody binds Ara h 2 with a K_{D} of between about 120 pM and about 10 nM.

In another aspect, the invention features an isolated antibody that binds to Ara h 2 (conglutin-7), or an antigen-binding fragment thereof, wherein the antibody comprises the following six CDRs: (a) an CDR-H1 comprising the amino acid sequence of GX1SX2SSYY (SEQ ID NO: 824), wherein X1 is D or G and X2 is I or M; (b) an CDR-H2 comprising the amino acid sequence of IFTX1GST (SEQ ID NO: 825), wherein X1 is S or T; (c) an CDR-H3 comprising the amino acid sequence of X1RDRRX2X3SX4DX5NWYWYFDL (SEQ ID NO: 826), wherein X1 is A or V, X2 is A or G, X3 is L or R, X4 is S or H, and X5 is G or S; (d) an CDR-L1 comprising the amino acid sequence of QTX1X2RN (SEQ ID NO: 827), wherein X1 is I or L and X2 is T or S; (e) an CDR-L2 comprising the amino acid sequence of GAS; and (f) an CDR-L3 comprising the amino acid sequence of QQSDNTPRT.

In another aspect, the invention features an isolated antibody that binds to Ara h 2 (conglutin-7), or an antigen-binding fragment thereof, wherein the antibody comprises the following six CDRs: (a) an CDR-H1 comprising the amino acid sequence of GDSISSYY (SEQ ID NO: 41); (b) an CDR-H2 comprising the amino acid sequence of IFTSGST (SEQ ID NO: 42); (c) an CDR-H3 comprising the amino acid sequence of ARDRRALSSDGNWYWYFDL (SEQ ID NO: 43); (d) an CDR-L1 comprising the amino acid sequence of QTITRN (SEQ ID NO: 44); (e) an CDR-L2 comprising the amino acid sequence of GAS (SEQ ID NO: 45); and (f) an CDR-L3 comprising the amino acid sequence of QQSDNTPRT (SEQ ID NO: 46). In some embodiments, the antibody comprises (a) a VH domain comprising an amino sequence having at least 90%, at least 95%, or at least 99% sequence identity to the amino acid sequence of SEQ ID NO: 47; (b) a VL domain comprising an amino acid sequence having at least 90%, at least 95%, or at least 99% identity to the amino acid sequence of SEQ ID NO: 48; or (c) a VH domain as in (a) and a VL domain as in (b). In some embodiments, the antibody further comprises the following VH domain FRs: (a) an FR-H1 comprising the amino acid sequence of ESGPRLVKPSETLSLTCIVS (SEQ ID NO: 49); (b) an FR-H2 comprising the amino acid sequence of WGWIRQPAGRGLEWIGR (SEQ ID NO: 50); (c) an FR-H3 comprising the amino acid sequence of TYNPSLKSRVSMSVETSKNQFSLTLTSVTAADTAVYFC (SEQ ID NO: 51); and (d) an FR-H4 comprising the amino acid sequence of WGRGTLVAVSS (SEQ ID NO: 52). In some embodiments, the VH domain comprises the amino acid sequence of SEQ ID NO: 47. In some embodiments, the antibody further comprises the following VL domain FRs: (a) an FR-L1 comprising the amino acid sequence of TQSPSSLSASVGDRVTITCRAS (SEQ ID NO: 53); (b) an FR-L2 comprising the amino acid sequence of LNWYQQKSGEAPKLLIY (SEQ ID NO: 54); (c) an FR-L3 comprising the amino acid sequence of ILQSGVPSRFTGSGSGTDFTLTISNLQPEDFASYSC (SEQ ID NO: 55); and (d) an FR-L4 comprising the amino acid sequence of GQGTKVEIK (SEQ ID NO: 56). In some embodiments, the VL domain comprises the amino acid sequence of SEQ ID NO: 48.

In another aspect, the invention features an isolated antibody that binds to Ara h 2, or an antigen-binding fragment thereof, wherein the antibody comprises (a) a VH domain comprising an amino acid sequence having at least 90%, at least 95%, or at least 99% sequence identity to the amino acid sequence of SEQ ID NO: 47 and (b) a VL domain comprising an amino acid sequence having at least 90%, at least 95%, or at least 99% sequence identity to the amino acid sequence of SEQ ID NO: 48. In some embodiments, the antibody comprises a VH domain comprising the amino acid sequence of SEQ ID NO: 47 and a VL domain comprising the amino acid sequence of SEQ ID NO: 48. In some embodiments, the antibody comprises (a) a heavy chain comprising the amino acid sequence of SEQ ID NO: 57 and (b) a light chain comprising the amino acid sequence of SEQ ID NO: 58.

In some embodiments of any of the previous aspects, the antibody binds Ara h 2 with a K_{D} of about 50 nM or less. In some embodiments, the K_{D} is measured by a surface plasmon resonance assay. In some embodiments, the antibody binds Ara h 2 with a K_{D} of between about 0.1 pM and about 40 nM. In some embodiments, the antibody binds Ara h 2 with a K_{D} of between about 120 pM and about 20 nM. In some embodiments, the antibody binds Ara h 2 with a K_{D} of between about 120 pM and about 10 nM.

In another aspect, the invention features an isolated antibody that binds to Ara h 2 (conglutin-7), or an antigen-binding fragment thereof, wherein the antibody comprises the following six CDRs: (a) an CDR-H1 comprising the amino acid sequence of GGSMSSYY (SEQ ID NO: 61); (b) an CDR-H2 comprising the amino acid sequence of IFTTGST (SEQ ID NO: 62); (c) an CDR-H3 comprising the amino acid sequence of VRDRRGRSHDSNWYWYFDL (SEQ ID NO: 63); (d) an CDR-L1 comprising the amino acid sequence of QTLSRN (SEQ ID NO: 64); (e) an CDR-L2 comprising the amino acid sequence of GAS (SEQ ID NO: 65); and (f) an CDR-L3 comprising the amino acid sequence of QQSDNTPRT (SEQ ID NO: 66). In some embodiments, the antibody comprises (a) a VH domain comprising an amino sequence having at least 90%, at least 95%, or at least 99% sequence identity to the amino acid sequence of SEQ ID NO: 67; (b) a VL domain comprising an amino acid sequence having at least 90%, at least 95%, or at least 99% identity to the amino acid sequence of SEQ ID NO: 68; or (c) a VH domain as in (a) and a VL domain as in (b). In some embodiments, the antibody further comprises the following VH domain FRs: (a) an FR-H1 comprising the amino acid sequence of QVQLQESGPGLVKPSETLSLTCTVS (SEQ ID NO: 69); (b) an FR-H2 comprising the amino acid sequence of WGWIRQPAGRGLEWIGR (SEQ ID NO: 70); (c) an FR-H3 comprising the amino acid sequence of IYNASLNSRVSMSVDTSKNQFSLKLTSVTAADTALYFC (SEQ ID NO: 71); and (d) an FR-H4 comprising the amino acid sequence of WGRGTLVTVSS (SEQ ID NO: 72). In some embodiments, the VH domain comprises the amino acid sequence of SEQ ID NO: 67. In some embodiments, the antibody further comprises the following VL domain FRs: (a) an FR-L1 comprising the amino acid sequence of RVIITCRAS (SEQ ID NO: 73); (b) an FR-L2 comprising the amino acid sequence of LNWYQQKPGEAPKLLIY (SEQ ID NO: 74); (c) an FR-L3 comprising the amino acid sequence of TLQSGVPSRFTGSGSGTDFTLIISGLQPEDFATYYC (SEQ ID NO: 75); and (d) an FR-L4 comprising the amino acid sequence of FGQGT (SEQ ID NO: 76). In some embodiments, the VL domain comprises the amino acid sequence of SEQ ID NO: 68.

In another aspect, the invention features an isolated antibody that binds to Ara h 2, or an antigen-binding fragment thereof, wherein the antibody comprises (a) a VH domain comprising an amino acid sequence having at least 90%, at least 95%, or at least 99% sequence identity to the amino acid sequence of SEQ ID NO: 67 and (b) a VL domain comprising an amino acid sequence having at least 90%, at least 95%, or at least 99% sequence identity to the amino acid sequence of SEQ ID NO: 68. In some embodiments, the antibody comprises a VH domain comprising the amino acid sequence of SEQ ID NO: 67 and a VL domain comprising the amino acid sequence of SEQ ID NO: 68. In some embodiments, the antibody comprises (a) a heavy chain comprising the amino acid sequence of SEQ ID NO: 77 and (b) a light chain comprising the amino acid sequence of SEQ ID NO: 78.

In some embodiments of any of the previous aspects, the antibody binds Ara h 2 with a K_{D} of about 50 nM or less. In some embodiments, the K_{D} is measured by a surface plasmon resonance assay. In some embodiments, the antibody binds Ara h 2 with a K_{D} of between about 0.1 pM and about 40 nM. In some embodiments, the antibody binds Ara h 2 with a K_{D} of between about 120 pM and about 20 nM. In some embodiments, the antibody binds Ara h 2 with a K_{D} of between about 120 pM and about 10 nM.

In some embodiments of any of the previous aspects, the antibody is capable of neutralizing Ara h 2.

In some embodiments of any of the previous aspects, the antibody is capable of blocking Ara h 2 binding with one or more binding partners.

In some embodiments of any of the previous aspects, the antibody is capable of blocking Ara h 2 binding with IgE.

In another aspect, the invention features an antibody that binds to the same epitope as the antibody of any one of the previous aspects. In some embodiments, whether the antibody binds to the same epitope or competes for binding to Ara h 2 is determined by an epitope binning assay.

In another aspect, the invention features an antibody that competes for binding to Ara h 2 with the antibody of any one of the previous aspects. In some embodiments, whether the antibody binds to the same epitope or competes for binding to Ara h 2 is determined by an epitope binning assay.

In some embodiments of any of the previous aspects, the antibody is monoclonal.

In some embodiments of any of the previous aspects, the antibody is humanized.

In some embodiments of any of the previous aspects, the antibody is an antibody fragment that binds Ara h 2. In some embodiments, the antibody fragment is selected from the group consisting of Fab, Fab′-SH, Fv, scFv, and (Fab′)₂ fragments.

In some embodiments of any of the previous aspects, the antibody is a full-length antibody. In some embodiments, the antibody is an IgG antibody. In some embodiments, the antibody is an IgG1 antibody. In some embodiments, the antibody is an IgG4 antibody. In some embodiments, the IgG4 antibody comprises an S228P mutation (EU numbering).

In some embodiments of any of the previous aspects, the antibody is a monospecific antibody.

In some embodiments of any of the previous aspects, the antibody is a multispecific antibody. In some embodiments, the multispecific antibody binds two or more sites on Ara h 2. In some embodiments, the two or more sites on Ara h 2 are within one or more epitope bins. In some embodiments, the one or more epitope bins are Bin 1, Bin 2, and/or Bin 3. In some embodiments, epitope Bin 1 includes the epitope for anti-Ara h2 antibodies T1 and/or T3. In some embodiments, epitope Bin 2 includes the epitope for anti-Ara h2 antibodies T4 and/or T5. In some embodiments, epitope Bin 3 does not include the epitope for anti-Ara h2 antibodies T1, T3, T4, and T5. In some embodiments, the antibody is a bispecific antibody.

In another aspect, the invention features a polynucleotide encoding an isolated antibody of any one of the previous aspects.

In another aspect, the invention features a vector comprising a polynucleotide encoding an isolated antibody of any one of the previous aspects.

In another aspect, the invention features a host cell comprising a vector comprising a polynucleotide encoding an isolated antibody of any one of the previous aspects.

In another aspect, the invention features a composition comprising the antibody of any one of the previous aspects. In some embodiments, the composition further comprises a pharmaceutically acceptable carrier, excipient, or diluent. In some embodiments, the composition is a pharmaceutical composition.

In some embodiments of any of the previous aspects, the antibody of any one of the previous aspects or the composition of any one of the previous aspects is for use as a medicament.

In some embodiments of any of the previous aspects, the antibody of any one of the previous aspects or the composition of any one of the previous aspects is for use in treating or delaying a peanut sensitivity. In some embodiments, the peanut sensitivity is a peanut allergy.

In another aspect, the invention features a method of producing the antibody of any one of the previous aspects, the method comprising culturing a host cell that comprises a vector comprising a polynucleotide encoding an isolated antibody of any one of the previous aspects. In some embodiments, the host cell is prokaryotic. In some embodiments, the host cell is Escherichia coli. In some embodiments, the host cell is eukaryotic. In some embodiments, the host cell is a 293 cell, a CHO cell, a yeast cell, or a plant cell.

In another aspect, the invention features a method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to the antibody of any one of the previous aspects. In some embodiments, the method further comprises administering an effective amount of a second therapeutic agent useful for diminishing an allergic reaction an allergic reaction against, a nut or a portion thereof or an extract thereof, or to Ara h 2 protein.

In another aspect, the invention features a method for detecting the presence of Ara h 2 neutralizing antibodies in a biological sample, comprising: (a) contacting a sample from a subject with a mixture comprising: (i) the antibody of any one of the previous aspects; and (ii) Ara h 2, or a fragment thereof; and (b) detecting the binding of antibodies present in the biological sample to Ara h 2, or a fragment thereof. In some embodiments, the antibody binding is detected using tandem biolayer interferometry. In some embodiments, the antibody from step (i) is bound to a solid support. In some embodiments, the Ara h 2, or a fragment thereof, is bound to a solid support. In some embodiments, antibody binding is detected using an enzyme-linked immunosorbent assay. In some embodiments, the subject is a human. In some embodiments, the subject has been diagnosed with a peanut sensitivity. In some embodiments, the subject has been diagnosed with a peanut allergy. In some embodiments, the subject has been previously treated or will be treated with peanut oral immunotherapy. In some embodiments, the sample is a blood or serum sample.

In another aspect, the invention features a method for detecting the presence of Ara h 2 neutralizing antibodies in a biological sample, comprising: (a) providing a sample from a subject; (b) contacting the sample with a mixture comprising: (i) the antibody of any one of the previous aspects; and (ii) Ara h 2, or a fragment thereof; and (c) detecting the binding of antibodies present in the biological sample to Ara h 2, or a fragment thereof. In some embodiments, the antibody binding is detected using tandem biolayer interferometry. In some embodiments, the antibody from step (i) is bound to a solid support. In some embodiments, the Ara h 2, or a fragment thereof, is bound to a solid support. In some embodiments, antibody binding is detected using an enzyme-linked immunosorbent assay. In some embodiments, the subject is a human. In some embodiments, the subject has been diagnosed with a peanut sensitivity. In some embodiments, the subject has been diagnosed with a peanut allergy. In some embodiments, the subject has been previously treated or will be treated with peanut oral immunotherapy. In some embodiments, the sample is a blood or serum sample.

In another aspect, the invention features a kit comprising the antibody or composition of any one of the previous aspects. In some embodiments, the kit further comprises instructions for use. In some embodiments, the kit further comprises one or more additional therapeutic agents (e.g., an antihistamine, epinephrine, a decongestant, or a corticosteroid).

Other features and advantages of the invention will be apparent from the following Detailed Description and the Claims.

### DETAILED DESCRIPTION OF THE INVENTION

Peanut-specific IgG blocking antibodies as well as combinations of such antibodies are disclosed to prevent IgE-mediated allergic reactions in affected patients. Administered subcutaneously, these antibodies provide protection to peanut allergic patients (or patients having peanut hypersensitivity) as a preventive measure. While this therapy would not induce long-term tolerance, as OIT does, it would prevent allergic reactions, which carry mortality and morbidity, without the risks inherent to OIT. Moreover, the approach of using passive protection is particularly important in our most vulnerable population, young children, for whom food allergies can carry additional social and psychological burdens.

The use of peanut-specific protective antibodies would provide passive protection to pediatric or adult patients with IgE-mediated peanut hypersensitivity, diagnosed by a combination of skin prick testing, specific IgE measurement, component IgE measurement, or oral food challenge. The medication may be administered as a subcutaneous injection, able to be administered at home or in an outpatient office setting by an Allergist. The antibodies described herein may prevent allergic reactions to accidental peanut exposures, direct or indirect, by providing clinical tolerance to an exposure of about 50-300 mg of peanut protein, which is roughly equivalent to 1 peanut. In some instances, the antibodies described herein may provide a clinical tolerance to an exposure to at least about 50-300 mg (e.g., about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 600 mg, about 700 mg, about 800 mg, about 900 mg, about 1 g, about 3 g, about 5 g, about 10 g, about 30 g, or more) of peanut protein.

The peanut-specific protective antibodies directed towards the immunodominant peanut antigen Ara h 2 as is disclosed herein were cloned from the circulation of pediatric patients who had sustained tolerance to peanut after OIT. From the same peanut OIT study, the suppression of basophil activation as a good biomarker of future tolerance is known. Furthermore, the suppression of basophils is mediated by IgG in the sera of tolerant patients, from whom the Ara h 2-specific antibodies described herein have been cloned. The protective antibodies were identified as those able to outcompete non-tolerant antibodies using biolayer light interferometry, as those have better antigen blocking capabilities.

Below we describe the invention as follows:
I. Definitions for understanding the specification;
II. Compositions and Methods including Therapy and Diagnostics; and
III. Examples.

### I. Definitions

The term “about” as used herein refers to the usual error range for the respective value readily known to the skilled person in this technical field. Reference to “about” a value or parameter herein includes (and describes) embodiments that are directed to that value or parameter per se.

The term “peanut allergen” refers to Ara h 2, and isoforms thereof or a protein with an amino acid sequence of at least 90%, preferably of at least 92%, further preferably of at least 95%, and again further preferably of at least 98% amino acid sequence identity with such a peanut allergen and isoform thereof. Preferably, the peanut allergen is Ara h 2 with an amino acid sequence of at least, 90%, 92%, 93%, 94%, 95%, 96%, 97%, 98% or even 99% identity with Ara h 2:

(SEQ ID NO: 821)
MAKLTILVALALFLLAAHASARQQWELQGDRRCQSQLERANLRPCEQHL
MQKIQRDEDSYGRDPYSPSQDPYSPSQDPDRRDPYSPSPYDRRGAGSSQ
HQERCCNELNEFENNQRCMCEALQQIMENQSDRLQGRQQEQQFKRELRN
LPQQCGLRAPQRCDLEVESGGRDRY

Signal sequence is amino acids 1-22. 4-hydroxyproline is located at positions 67, 74, and 86.

Allergenic fragments of Ara h 2 are also considered peanut allergens and may be identified according to standard methods.

The terms “anti-Ara h 2 antibody,” “an antibody that binds to Ara h 2,” and “an antibody that specifically binds to Ara h 2” refer to an antibody that is capable of binding Ara h 2 with sufficient affinity such that the antibody is useful as a preventative, diagnostic, and/or therapeutic agent in targeting Ara h 2. In one embodiment, the extent of binding of an anti-Ara h 2 antibody to an unrelated, non-Ara h 2 protein is less than about 10% of the binding of the antibody to Ara h 2 as measured, e.g., by a radioimmunoassay (RIA). In certain embodiments, an antibody that binds to Ara h 2 has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). In certain embodiments, an antibody that binds to Ara h 2 has a K_{D} of between about 0.0001 nM and about 100 nM. In certain embodiments, an anti-Ara h 2 antibody binds to an epitope of Ara h 2 that is conserved among Ara h 2 from different peanut species.

The term “antibody” as used herein in the broadest sense encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody” can refer, for example, to a glycoprotein comprising at least two heavy chains (HCs) and two light chains (LCs) inter-connected by disulfide bonds, or an antigen binding portion thereof. Each heavy chain is comprised of a heavy chain variable region (VH) and a heavy chain constant region (CH). The heavy chain constant region may be comprised of three domains, CH1, CH2, and/or CH3. Each light chain is comprised of a light chain variable region (VL) and a light chain constant region (CL). The VH and VL regions can be further subdivided into regions of hypervariability, termed “complementarity determining regions” (CDRs), interspersed with regions that are more conserved, termed “framework regions” (FRs). Each VH and VL may be composed, for example, of three CDRs and four FRs, arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “full-length antibody,” “intact antibody,” and “whole antibody” are used herein interchangeably to refer to an antibody having a structure substantially similar to a native antibody structure or having heavy chains that contain an Fc region as defined herein.

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368 (6474): 856-859); Lonberg, N. (1994) Handbook of Experimental Pharmacology 113:49-101; Lonberg, N. and Huszar, D. (1995) Intern. Rev. Immunol. Vol. 13:65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., humanized antibodies).

The term “monoclonal antibody,” as used herein, refers to an antibody which displays a single binding specificity and affinity for a particular epitope. Accordingly, the term “human monoclonal antibody,” or “HuMab,” refers to an antibody which displays a single binding specificity, and which has variable and constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

An “antibody fragment” refers to a molecule other than an intact antibody that comprises a portion of an intact antibody that specifically binds to the antigen (e.g., an Ara h 2 protein described aboveto which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. These antibody fragments are obtained using conventional techniques, and the fragments are screened for utility in the same manner as are intact antibodies. Antibody fragments can be produced by recombinant DNA techniques, or by enzymatic or chemical cleavage of intact immunoglobulins.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described below.

The term “K_{D} is measured by a surface plasmon resonance assay,” when used in the context of the claims, means that the K_{D} is measured according the method described in Example 2 which measures kinetic parameters for binding of anti-Ara h 2 antibodies to Ara h 2 described in Patil et al., J. Allergy Clin Immunol. 136(1):125-134 (2015). Typically, the antibodies disclosed herein bind to Ara h 2 with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower when determined by surface plasmon resonance (SPR) technology in a BIACORE 3000 instrument using recombinant Ara h 2 as the analyte and the antibody as the ligand.

The term “EC50,” as used herein, refers to the concentration of an antibody or an antigen-binding portion thereof, which induces a response, either in an in vivo or an in vitro assay, such as neutralization of Ara h 2 (e.g., blocking Ara h 2 binding with a binding partner (e.g., an IgE antibody)) as is described herein, which is 50% of the maximal response (i.e., halfway between the maximal response and the baseline).

The terms “effective amount,” “effective dose,” and “effective dosage” as used herein are defined as an amount sufficient to achieve, or at least partially achieve, the desired effect. The term “therapeutically effective dose” or “therapeutically effective amount” is defined as an amount sufficient to prevent, cure, or at least partially arrest, the allergic reaction and its complications in a patient already suffering from an allergic reaction to peanut exposure or at risk to being exposed to Ara h 2 or an allergenic fragment thereof. Amounts effective for this use will depend upon the severity of the allergic reaction being treated and the general state of the patient's own immune system.

The term “epitope” or “antigenic determinant” refers to a site on an antigen to which an immunoglobulin or antibody specifically binds on Ara h 2. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography, cryo-electron microscopy, and 2-dimensional nuclear magnetic resonance. See, for example, Epitope Mapping Protocols in Methods in Molecular Biology, Vol. 66, G. E. Morris, Ed. (1996). Epitopes can also be defined by point mutations in the target protein (e.g., Ara h 2 or an allergic-inducing fragment thereof), which affect the binding of the antibody (e.g., monoclonal antibody).

The term “host cell,” as used herein, is intended to refer to a cell into which an expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

An “isolated antibody” is one which has been identified and separated and/or recovered from a component of its natural environment and/or is substantially free of other antibodies having different antigenic specificities (e.g., an isolated antibody that specifically binds to Ara h 2 is substantially free of antibodies that specifically bind antigens other than Ara h 2). Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie™ blue or, preferably, silver stain. Isolated antibody includes the antibody in situ within recombinant cells since at least one component of the antibody's natural environment will not be present. Similarly, isolated antibody includes the antibody in medium around recombinant cells. Ordinarily, however, isolated antibody will be prepared by at least one purification step.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid,” as used herein in reference to nucleic acids molecules encoding antibodies or antibody portions (e.g., VH, VL, CDRs) that bind to Ara h 2, is intended to refer to a nucleic acid molecule in which the nucleotide sequences encoding the antibody or antibody portion are free of other nucleotide sequences encoding antibodies that bind antigens other than Ara h 2, which other sequences may naturally flank the nucleic acid in human genomic DNA.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program is registered under U.S. Copyright Registration No. TXU510087.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the terms “specific binding,” “selective binding,” “selectively binds,” and “specifically binds,” refer to antibody binding to an epitope on a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument, which can be performed, for example, using recombinant Ara h 2 as the analyte and the antibody as the ligand. In some embodiments, binding by the antibody to the predetermined antigen is with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

A “subject,” a “patient,” or an “individual” is typically a human such as an adult, a child, or an infant.

As used herein, “administering” is meant a method of giving a dosage of a compound (e.g., an anti-Ara h 2 antibody or a nucleic acid encoding an anti-Ara h 2 antibody) or a composition (e.g., a pharmaceutical composition, e.g., a pharmaceutical composition including an anti-Ara h 2 antibody) to a subject. Preferably, the antibodies described herein are administered subcutaneously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered, and the severity of the peanut allergy being treated). Preferably, the antibody or a combination of antibodies (for example, a combination of the T3 and T4 antibodies disclosed herein) are administered as a subcutaneous injection.

As used herein, the term “vector” is meant to include, but is not limited to, a nucleic acid molecule (e.g., a nucleic acid molecule that is capable of transporting another nucleic acid to which it has been linked), a virus (e.g., a lentivirus or an adenovirus, e.g., a recombinant adeno-associated virus (rAAV)), cationic lipid (e.g., liposome), cationic polymer (e.g., polysome), virosome, nanoparticle, or dentrimer. Accordingly, one type of vector is a viral vector, wherein additional DNA segments (e.g., transgenes, e.g., transgenes encoding the heavy and/or light chain genes of an anti-Ara h 2 antibody) may be ligated into the viral genome, and the viral vector may then be administered (e.g., by electroporation, e.g., electroporation into muscle tissue) to the subject in order to allow for transgene expression in a manner analogous to gene therapy. Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids.

### II. Compositions and Methods

In one aspect, the invention is based, in part, on anti-Ara h 2 antibodies. Such antibodies are useful, for example, for treating a subject having, or at risk of developing, a peanut allergy following exposure, direct or indirect, to a peanut allergen such as Ara h 2.

### A. Anti-Ara h 2 Antibodies

The invention provides isolated antibodies (e.g., any of the antibodies described herein) that bind to Ara h 2.

Accordingly, in one aspect, the invention provides isolated antibody that specifically binds to Ara h 2.

In another aspect, the antibody includes the following complementarity determining regions (CDRs): (a) a CDR-H1 comprising the amino acid sequence of GFTFRDYS; (b) a CDR-H2 comprising the amino acid sequence of IRFDGTTK; (c) a CDR-H3 comprising the amino acid sequence of X₁KDNGWRAFDH (SEQ ID NO: 822), wherein X₁ is V or A; (d) a CDR-L1 comprising the amino acid sequence of QSLLHRNGYIY; (e) a CDR-L2 comprising the amino acid sequence of FVS; and (f) a CDR-L3 comprising the amino acid sequence of MQX₁LEX₂PWT (SEQ ID NO: 823), wherein X₁ is S or A and X₂ is N or T, or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of the aforementioned sequences.

In another aspect, the invention provides isolated antibody that specifically binds to Ara h 2. In some instances, the antibody includes the following complementarity determining regions (CDRs): (a) a CDR-H1 comprising the amino acid sequence of GX₁SX₂SSYY (SEQ ID NO: 824), wherein X₁ is D or G and X₂ is I or M; (b) a CDR-H2 comprising the amino acid sequence of IFTX₁GST (SEQ ID NO: 825), wherein X₁ is S or T; (c) a CDR-H3 comprising the amino acid sequence of X₁RDRRX₂X₃SX₄DX₅NWYWYFDL (SEQ ID NO: 826), wherein X₁ is A or V, X₂ is A or G, X₃ is L or R, X₄ is S or H, and X₅ is G or S; (d) a CDR-L1 comprising the amino acid sequence of QTX₁X₂RN (SEQ ID NO: 827), wherein X₁ is I or L and X₂ is T or S; (e) a CDR-L2 comprising the amino acid sequence of GAS; and (f) a CDR-L3 comprising the amino acid sequence of QQSDNTPRT, or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of the aforementioned sequences.

Examples of other antibodies include T1 (also referred to as V1), T3 (also referred to as V2), T4 (also referred to as V3), and T5 (also referred to as V4) as described herein, as well as the anti-Ara h 2 antibodies V5-V41 described herein.

### T1

In some instances, the antibody includes one or more of the following six CDRs: (a) a CDR-H1 comprising the amino acid sequence of GFTFRDYS (SEQ ID NO: 1); (b) a CDR-H2 comprising the amino acid sequence of IRFDGTTK (SEQ ID NO: 2); (c) a CDR-H3 comprising the amino acid sequence of VKDNGWRAFDH (SEQ ID NO: 3); (d) a CDR-L1 comprising the amino acid sequence of QSLLHRNGYIY (SEQ ID NO: 4); (e) a CDR-L2 comprising the amino acid sequence of FVS (SEQ ID NO: 5); and (f) a CDR-L3 comprising the amino acid sequence of MQSLENPWT (SEQ ID NO: 6), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 1-6.

In some instances, the antibody includes the following six CDRs: (a) a CDR-H1 comprising the amino acid sequence of GFTFRDYS (SEQ ID NO: 1); (b) a CDR-H2 comprising the amino acid sequence of IRFDGTTK (SEQ ID NO: 2); (c) a CDR-H3 comprising the amino acid sequence of VKDNGWRAFDH (SEQ ID NO: 3); (d) a CDR-L1 comprising the amino acid sequence of QSLLHRNGYIY (SEQ ID NO: 4); (e) a CDR-L2 comprising the amino acid sequence of FVS (SEQ ID NO: 5); and (f) a CDR-L3 comprising the amino acid sequence of MQSLENPWT (SEQ ID NO: 6).

In some instances, the antibody has a VH domain comprising an amino acid sequence having at least 90% sequence identity (e.g., at least 91%, 92%, 93%, 94%, 95%, 96%, 97%, 98%, or 99% sequence identity) to, or the sequence of, VESGGGVVQPGASLRLSCVASGFTFRDYSMHWVRQAPGKGLEWMSFIRFDGTTKDYRESVKGRFIISR DDSKNTLYLQMTSLRPEDTARYYCVKDNGWRAFDHWGQGALVIVSS (SEQ ID NO: 7); and/or a VL domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 8)
EIVMTQSPLSLPVTPGEAASISCRSSQSLLHRNGYIYLDWYLQRPGQSP
QLLISFVSKRASGAPDRFSGSGSGTDFTLTISRVEAEDFGVYFCMQSLE
NPWTFGP.

In some instances, the antibody comprises the following VH domain framework regions (FRs): (a) an FR-H1 comprising the amino acid sequence of VESGGGVVQPGASLRLSCVAS (SEQ ID NO: 9); (b) an FR-H2 comprising the amino acid sequence of MHWVRQAPGKGLEWMSF (SEQ ID NO: 10); (c) an FR-H3 comprising the amino acid sequence of DYRESVKGRFIISRDDSKNTLYLQMTSLRPEDTARYYC (SEQ ID NO: 11); and (d) an FR-H4 comprising the amino acid sequence of WGQGALVIVSS (SEQ ID NO: 12).

In some instances, the antibody comprises the following VL domain FRs: (a) an FR-L1 comprising the amino acid sequence of EIVMTQSPLSLPVTPGEAASISCRSS (SEQ ID NO: 13); (b) an FR-L2 comprising the amino acid sequence of LDWYLQRPGQSPQLLIS (SEQ ID NO: 14); (c) an FR-L3 comprising the amino acid sequence of KRASGAPDRFSGSGSGTDFTLTISRVEAEDFGVYFC (SEQ ID NO: 15); and (d) an FR-L4 comprising the amino acid sequence of FGP (SEQ ID NO: 16).

In some instances, the antibody includes a heavy chain and a light chain sequence, wherein: (a) the heavy chain comprises the amino acid sequence: VESGGGVVQPGASLRLSCVASGFTFRDYSMHWVRQAPGKGLEWMSFIRFDGTTKDYRESVKGRFIISR DDSKNTLYLQMTSLRPEDTARYYCVKDNGWRAFDHWGQGALVIVSSKGPSVFPLAPSSKSTSGGTAAL GXLVKDYFPEPVTVSWNSGALTSGVHTFPAVLQSSGLYSLSSVVTVPSSSLGTQTYICNVNHKPSNTKVD KXVEPKSCDKTHTCPPCPAPELLGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTISKAKGQPREPQVYTL PPSRDEXTKNQVSLTCLVKGFYPSDIAVEWESNGQPENNYKTTPPVLDSDGSFFLYSKLTVDKSRWQQG NVFSCSVMHEALHNHYTQKSLSLSPGK (SEQ ID NO: 17); and (b) the light chain comprises the amino acid sequence:

(SEQ ID NO: 18)
EIVMTQSPLSLPVTPGEAASISCRSSQSLLHRNGYIYLDWYLQRPGQSP
QLLISFVSKRASGAPDRFSGSGSGTDFTLTISRVEAEDFGVYFCMQSLE
NPWTFGPAPSVFIFPPSDEQLKSGTASVVCLLNNFYPREAKVQWKVDNA
LQSGNSQESVTEQDSKDSTYSLSSTLTLSKADYEKHKVYACEVTHQGLS
SPVTKSFNRGEC.

In some instances, the antibody includes a heavy chain comprising a nucleic acid sequence of SEQ ID NO: 19. In some instances, the antibody includes a light chain comprising a nucleic acid sequence of SEQ ID NO: 20.

### T3

In some instances, the antibody includes one or more of the following six CDRs: (a) a CDR-H1 comprising the amino acid sequence of GFTFRDYS (SEQ ID NO: 21); (b) a CDR-H2 comprising the amino acid sequence of IRFDGTTK (SEQ ID NO: 22); (c) a CDR-H3 comprising the amino acid sequence of AKDNGWRAFDH (SEQ ID NO: 23); (d) a CDR-L1 comprising the amino acid sequence of QSLLHRNGYIY (SEQ ID NO: 24); (e) a CDR-L2 comprising the amino acid sequence of FVS (SEQ ID NO: 25); and (f) a CDR-L3 comprising the amino acid sequence of MQALETPWT (SEQ ID NO: 26), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 21-26. In some instances, the antibody includes the following six CDRs: (a) a CDR-H1 comprising the amino acid sequence of GFTFRDYS (SEQ ID NO: 21); (b) a CDR-H2 comprising the amino acid sequence of IRFDGTTK (SEQ ID NO: 22); (c) a CDR-H3 comprising the amino acid sequence of AKDNGWRAFDH (SEQ ID NO: 23); (d) a CDR-L1 comprising the amino acid sequence of QSLLHRNGYIY (SEQ ID NO: 24); (e) a CDR-L2 comprising the amino acid sequence of FVS (SEQ ID NO: 25); and (f) a CDR-L3 comprising the amino acid sequence of MQALETPWT (SEQ ID NO: 26).

In some instances, the antibody has a VH domain comprising an amino acid sequence having at least 90% sequence identity (e.g., at least 91%, 92%, 93%, 94%, 95%, 96%, 97%, 98%, or 99% sequence identity) to, or the sequence of, GGGVVQPGTSLRLSCVASGFTFRDYSMHWVRQAPGKGLEWVSFIRFDGTTKDYRDSVKGRFIISRDDSK NTLYLQMTSLRVEDTALYYCAKDNGWRAFDHWGQGALVTVSS (SEQ ID NO: 27); and/or a VL domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 28)
VMTQSPLSLPVTPGEAASISCRSSQSLLHRNGYIYLDWYLQRPGQSPQL
LISFVSKRASGAPDRFSGSGSGTDFTLTISRVEAEDFGVYFCMQALETP
WTFGPGTKLEIK.

In some instances, the antibody comprises the following VH domain FRs: (a) an FR-H1 comprising the amino acid sequence of GGGVVQPGTSLRLSCVAS (SEQ ID NO: 29); (b) an FR-H2 comprising the amino acid sequence of MHWVRQAPGKGLEWVSF (SEQ ID NO: 30); (c) an FR-H3 comprising the amino acid sequence of DYRDSVKGRFIISRDDSKNTLYLQMTSLRVEDTALYYC (SEQ ID NO: 31); and (d) an FR-H4 comprising the amino acid sequence of WGQGALVTVSS (SEQ ID NO: 32).

In some instances, the antibody comprises the following VL domain FRs: (a) an FR-L1 comprising the amino acid sequence of VMTQSPLSLPVTPGEAASISCRSS (SEQ ID NO: 33); (b) an FR-L2 comprising the amino acid sequence of LDWYLQRPGQSPQLLIS (SEQ ID NO: 34); (c) an FR-L3 comprising the amino acid sequence of KRASGAPDRFSGSGSGTDFTLTISRVEAEDFGVYFC (SEQ ID NO: 35); and (d) an FR-L4 comprising the amino acid sequence of FGPGTKLEIK (SEQ ID NO: 36).

In some instances, the antibody includes a heavy chain and a light chain sequence, wherein: (a) the heavy chain comprises the amino acid sequence: GGGVVQPGTSLRLSCVASGFTFRDYSMHWVRQAPGKGLEWVSFIRFDGTTKDYRDSVKGRFIISRDDSK NTLYLQMTSLRVEDTALYYCAKDNGWRAFDHWGQGALVTVSSKGPSVFPLAPSSKSTSGGTAALGXLV KDYFPEPVTVSWNSGALTSGVHTFPAVLQSSGLYSLSSVVTVPSSSLGTQTYICNVNHKPSNTKVDKXVE PKSCDKTHTCPPCPAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKGQPREPQVYTLPPSR DEXTKNQVSLTCLVKGFYPSDIAVEWESNGQPENNYKTTPPVLDSDGSFFLYSKLTVDKSRWQQGNVFS CSVMHEALHNHYTQKSLSLSPGK (SEQ ID NO: 37); and (b) the light chain comprises the amino acid sequence:

(SEQ ID NO: 38)
VMTQSPLSLPVTPGEAASISCRSSQSLLHRNGYIYLDWYLQRPGQSPQL
LISFVSKRASGAPDRFSGSGSGTDFTLTISRVEAEDFGVYFCMQALETP
WTFGPGTKLEIKAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

In some instances, the antibody includes a heavy chain comprising a nucleic acid sequence of SEQ ID NO: 39. In some instances, the antibody includes a light chain comprising a nucleic acid sequence of SEQ ID NO: 40.

### T4

In some instances, the antibody includes one or more of the following six CDRs: (a) a CDR-H1 comprising the amino acid sequence of GDSISSYY (SEQ ID NO: 41); (b) a CDR-H2 comprising the amino acid sequence of IFTSGST (SEQ ID NO: 42); (c) a CDR-H3 comprising the amino acid sequence of ARDRRALSSDGNWYWYFDL (SEQ ID NO: 43); (d) a CDR-L1 comprising the amino acid sequence of QTITRN (SEQ ID NO: 44); (e) a CDR-L2 comprising the amino acid sequence of GAS (SEQ ID NO: 45); and (f) a CDR-L3 comprising the amino acid sequence of QQSDNTPRT (SEQ ID NO: 46), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 41-46. In some instances, the antibody includes the following six CDRs: (a) a CDR-H1 comprising the amino acid sequence of GDSISSYY (SEQ ID NO: 41); (b) a CDR-H2 comprising the amino acid sequence of IFTSGST (SEQ ID NO: 42); (c) a CDR-H3 comprising the amino acid sequence of ARDRRALSSDGNWYWYFDL (SEQ ID NO: 43); (d) a CDR-L1 comprising the amino acid sequence of QTITRN (SEQ ID NO: 44); (e) a CDR-L2 comprising the amino acid sequence of GAS (SEQ ID NO: 45); and (f) a CDR-L3 comprising the amino acid sequence of QQSDNTPRT (SEQ ID NO: 46).

In some instances, the antibody has a VH domain comprising an amino acid sequence having at least 90% sequence identity (e.g., at least 91%, 92%, 93%, 94%, 95%, 96%, 97%, 98%, or 99% sequence identity) to, or the sequence of, ESGPRLVKPSETLSLTCIVSGDSISSYYWGWIRQPAGRGLEWIGRIFTSGSTTYNPSLKSRVSMSVETSK NQFSLTLTSVTAADTAVYFCARDRRALSSDGNWYWYFDLWGRGTLVAVSS (SEQ ID NO: 47); and/or a VL domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 48)
TQSPSSLSASVGDRVTITCRASQTITRNLNWYQQKSGEAPKLLIYGASI
LQSGVPSRFTGSGSGTDFTLTISNLQPEDFASYSCQQSDNTPRTFGQGT
KVEIK.

In some instances, the antibody comprises the following VH domain FRs: (a) an FR-H1 comprising the amino acid sequence of ESGPRLVKPSETLSLTCIVS (SEQ ID NO: 49); (b) an FR-H2 comprising the amino acid sequence of WGWIRQPAGRGLEWIGR (SEQ ID NO: 50); (c) an FR-H3 comprising the amino acid sequence of TYNPSLKSRVSMSVETSKNQFSLTLTSVTAADTAVYFC (SEQ ID NO: 51); and (d) an FR-H4 comprising the amino acid sequence of WGRGTLVAVSS (SEQ ID NO: 52).

In some instances, the antibody comprises the following VL domain FRs: (a) an FR-L1 comprising the amino acid sequence of TQSPSSLSASVGDRVTITCRAS (SEQ ID NO: 53); (b) an FR-L2 comprising the amino acid sequence of LNWYQQKSGEAPKLLIY (SEQ ID NO: 54); (c) an FR-L3 comprising the amino acid sequence of ILQSGVPSRFTGSGSGTDFTLTISNLQPEDFASYSC (SEQ ID NO: 55); and (d) an FR-L4 comprising the amino acid sequence of GQGTKVEIK (SEQ ID NO: 56).

In some instances, the antibody includes a heavy chain and a light chain sequence, wherein: (a) the heavy chain comprises the amino acid sequence: ESGPRLVKPSETLSLTCIVSGDSISSYYWGWIRQPAGRGLEWIGRIFTSGSTTYNPSLKSRVSMSVETSK NQFSLTLTSVTAADTAVYFCARDRRALSSDGNWYWYFDLWGRGTLVAVSSKGPSVFPLAPSSKSTSGG TAALGXLVKDYFPEPVTVSWNSGALTSGVHTFPAVLQSSGLYSLSSVVTVPSSSLGTQTYICNVNHKPSN TKVDKXVEPKSCDKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIEKTISKAKGQPREPQ VYTLPPSRDEXTKNQVSLTCLVKGFYPSDIAVEWESNGQPENNYKTTPPVLDSDGSFFLYSKLTVDKSR WQQGNVFSCSVMHEALHNHYTQKSLSLSPGK (SEQ ID NO: 57); and (b) the light chain comprises the amino acid sequence:

(SEQ ID NO: 58)
TQSPSSLSASVGDRVTITCRASQTITRNLNWYQQKSGEAPKLLIYGASI
LQSGVPSRFTGSGSGTDFTLTISNLQPEDFASYSCQQSDNTPRTFGQGT
KVEIKAPSVFIFPPSDEQLKSGTASVVCLLNNFYPREAKVQWKVDNALQ
SGNSQESVTEQDSKDSTYSLSSTLTLSKADYEKHKVYACEVTHQGLSSP
VTKSFNRGEC.

In some instances, the antibody includes a heavy chain comprising a nucleic acid sequence of SEQ ID NO: 59. In some instances, the antibody includes a light chain comprising a nucleic acid sequence of SEQ ID NO: 60.

### T5

In some instances, the antibody includes one or more of the following six CDRs: (a) a CDR-H1 comprising the amino acid sequence of GGSMSSYY (SEQ ID NO: 61); (b) a CDR-H2 comprising the amino acid sequence of IFTTGST (SEQ ID NO: 62); (c) a CDR-H3 comprising the amino acid sequence of VRDRRGRSHDSNWYWYFDL (SEQ ID NO: 63); (d) a CDR-L1 comprising the amino acid sequence of QTLSRN (SEQ ID NO: 64); (e) a CDR-L2 comprising the amino acid sequence of GAS (SEQ ID NO: 65); and (f) a CDR-L3 comprising the amino acid sequence of QQSDNTPRT (SEQ ID NO: 66), or a combination of one or more of the above CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 61-66. In some instances, the antibody includes the following six CDRs: (a) a CDR-H1 comprising the amino acid sequence of GGSMSSYY (SEQ ID NO: 61); (b) a CDR-H2 comprising the amino acid sequence of IFTTGST (SEQ ID NO: 62); (c) a CDR-H3 comprising the amino acid sequence of VRDRRGRSHDSNWYWYFDL (SEQ ID NO: 63); (d) a CDR-L1 comprising the amino acid sequence of QTLSRN (SEQ ID NO: 64); (e) a CDR-L2 comprising the amino acid sequence of GAS (SEQ ID NO: 65); and (f) a CDR-L3 comprising the amino acid sequence of QQSDNTPRT (SEQ ID NO: 66).

In some instances, the antibody has a VH domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 67)
QVQLQESGPGLVKPSETLSLTCTVSGGSMSSYYWGWIRQPAGRGLEWIG
RIFTTGSTIYNASLNSRVSMSVDTSKNQFSLKLTSVTAADTALYFCVRD
RRGRSHDSNWYWYFDLWGRGTLVTVSS;

and/or a VL domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 68)
RVIITCRASQTLSRNLNWYQQKPGEAPKLLIYGASTLQSGVPSRFTGSG
SGTDFTLIISGLQPEDFATYYCQQSDNTPRTFGQGT.

In some instances, the antibody comprises the following VH domain FRs: (a) an FR-H1 comprising the amino acid sequence of QVQLQESGPGLVKPSETLSLTCTVS (SEQ ID NO: 69); (b) an FR-H2 comprising the amino acid sequence of WGWIRQPAGRGLEWIGR (SEQ ID NO: 70); (c) an FR-H3 comprising the amino acid sequence of IYNASLNSRVSMSVDTSKNQFSLKLTSVTAADTALYFC (SEQ ID NO: 71); and (d) an FR-H4 comprising the amino acid sequence of WGRGTLVTVSS (SEQ ID NO: 72).

In some instances, the antibody comprises the following VL domain FRs: (a) an FR-L1 comprising the amino acid sequence of RVIITCRAS (SEQ ID NO: 73); (b) an FR-L2 comprising the amino acid sequence of LNWYQQKPGEAPKLLIY (SEQ ID NO: 74); (c) an FR-L3 comprising the amino acid sequence of TLQSGVPSRFTGSGSGTDFTLIISGLQPEDFATYYC (SEQ ID NO: 75); and (d) an FR-L4 comprising the amino acid sequence of FGQGT (SEQ ID NO: 76).

In some instances, the antibody includes a heavy chain and a light chain sequence, wherein: (a) the heavy chain comprises the amino acid sequence: QVQLQESGPGLVKPSETLSLTCTVSGGSMSSYYWGWIRQPAGRGLEWIGRIFTTGSTIYNASLNSRVSM SVDTSKNQFSLKLTSVTAADTALYFCVRDRRGRSHDSNWYWYFDLWGRGTLVTVSSKGPSVFPLAPSS KSTSGGTAALGXLVKDYFPEPVTVSWNSGALTSGVHTFPAVLQSSGLYSLSSVVTVPSSSLGTQTYICNV NHKPSNTKVDKXVEPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSNKALPAPIEKTISKAKG QPREPQVYTLPPSRDEXTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 77); and (b) the light chain comprises the amino acid sequence:

(SEQ ID NO: 78)
RVIITCRASQTLSRNLNWYQQKPGEAPKLLIYGASTLQSGVPSRFTGSG
SGTDFTLIISGLQPEDFATYYCQQSDNTPRTFGQGTAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGEC.

In some instances, the antibody includes a heavy chain comprising a nucleic acid sequence of SEQ ID NO: 79. In some instances, the antibody includes a light chain comprising a nucleic acid sequence of SEQ ID NO: 80.

### V5-V41

In some instances, the antibody includes sequences corresponding to any one of the following antibodies: V5 (SEQ ID NOs: 81-100), V6 (SEQ ID NOs: 101-120), V7 (SEQ ID NOs: 121-140), V8 (SEQ ID NOs: 141-160), V9 (SEQ ID NOs: 161-180), V10 (SEQ ID NOs: 181-200), V11 (SEQ ID NOs: 201-120), V12 (SEQ ID NOs: 221-240), V13 (SEQ ID NOs: 241-260), V14 (SEQ ID NOs: 261-280), V15 (SEQ ID NOs: 281-300), V16 (SEQ ID NOs: 301-320), V17 (SEQ ID NOs: 321-340), V18 (SEQ ID NOs: 341-360), V19 (SEQ ID NOs: 361-380), V20 (SEQ ID NOs: 381-300), V21 (SEQ ID NOs: 401-420), V22 (SEQ ID NOs: 421-440), V23 (SEQ ID NOs: 441-460), V24 (SEQ ID NOs: 461-480), V25 (SEQ ID NOs: 481-500), V26 (SEQ ID NOs: 501-420), V27 (SEQ ID NOs: 521-540), V28 (SEQ ID NOs: 541-560), V29 (SEQ ID NOs: 561-580), V30 (SEQ ID NOs: 581-600), V31 (SEQ ID NOs: 601-620), V32 (SEQ ID NOs: 621-640), V33 (SEQ ID NOs: 641-660), V34 (SEQ ID NOs: 661-680), V35 (SEQ ID NOs: 681-700), V36 (SEQ ID NOs: 701-720), V37 (SEQ ID NOs: 721-740), V38 (SEQ ID NOs: 741-760), V39 (SEQ ID NOs: 761-780), V40 (SEQ ID NOs: 781-700), and V41 (SEQ ID NOs: 801-820).

In some instances, the antibody comprises a CDR-H1 comprising the amino acid sequence of SEQ ID NOs: 81, 101, 121, 141, 161, 181, 201, 221, 241, 261, 281, 301, 321, 341, 361, 381, 401, 421, 441, 461, 481, 501, 521, 541, 561, 581, 601, 621, 641, 661, 681, 701, 721, 741, 761, 781, or 801, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody comprises a CDR-H2 comprising the amino acid sequence of SEQ ID NOs: 82, 102, 122, 142, 162, 182, 202, 222, 242, 262, 282, 302, 322, 342, 362, 382, 402, 422, 442, 462, 482, 502, 522, 542, 562, 582, 602, 622, 642, 662, 682, 702, 722, 742, 762, 782, or 802, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody comprises a CDR-H3 comprising the amino acid sequence of SEQ ID NOs: 83, 103, 123, 143, 163, 183, 203, 223, 243, 263, 283, 303, 323, 343, 363, 383, 403, 423, 443, 463, 483, 503, 523, 543, 563, 583, 603, 623, 643, 663, 683, 703, 723, 743, 763, 783, or 803, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody comprises a CDR-L1 comprising the amino acid sequence of SEQ ID NOs: 84, 104, 124, 144, 164, 184, 204, 224, 244, 264, 284, 304, 324, 344, 364, 384, 404, 424, 444, 464, 484, 504, 524, 544, 564, 584, 604, 624, 644, 664, 684, 704, 724, 744, 764, 784, or 804, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody comprises a CDR-L2 comprising the amino acid sequence of SEQ ID NOs: 85, 105, 125, 145, 165, 185, 205, 225, 245, 265, 285, 305, 325, 345, 365, 385, 405, 425, 445, 465, 485, 505, 525, 545, 565, 585, 605, 625, 645, 665, 685, 705, 725, 745, 765, 785, or 805, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody comprises a CDR-L3 comprising the amino acid sequence of SEQ ID NOs: 86, 106, 126, 146, 166, 186, 206, 226, 246, 266, 286, 306, 326, 346, 366, 386, 406, 426, 446, 466, 486, 506, 526, 546, 566, 586, 606, 626, 646, 666, 686, 706, 726, 746, 766, 786, or 806, or variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity). In some instances, the antibody includes one or more of six CDRs, or a combination of one or more of the CDRs and one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) of any of the following antibodies: V5-V41. In some instances, the antibody includes six CDRs from any one of the following antibodies: V5-V41.

In some instances, the antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NOs: 87, 107, 127, 147, 167, 187, 207, 227, 247, 267, 287, 307, 327, 347, 367, 387, 407, 427, 447, 467, 487, 507, 527, 547, 567, 587, 607, 627, 647, 667, 687, 707, 727, 747, 767, 787, or 807. In some instances, the antibody has a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NOs: 88, 108, 128, 148, 168, 188, 208, 228, 248, 268, 288, 308, 328, 348, 368, 388, 408, 428, 448, 468, 488, 508, 528, 548, 568, 588, 608, 628, 648, 668, 688, 708, 728, 748, 768, 788, or 808. In some instances, the antibody includes a VH and a VL sequence from any one of the following antibodies: V5-V41.

In some instances, the antibody includes an FR-H1 comprising an amino acid sequence any one of SEQ ID NOs: 89, 109, 129, 149, 169, 189, 209, 229, 249, 269, 289, 309, 329, 349, 369, 389, 409, 429, 449, 469, 489, 509, 529, 549, 569, 589, 609, 629, 649, 669, 689, 709, 729, 749, 769, 789, or 809. In some instances, the antibody includes an FR-H2 comprising an amino acid sequence any one of SEQ ID NOs: 90, 110, 130, 150, 170, 190, 210, 230, 250, 270, 290, 310, 330, 350, 370, 390, 410, 430, 450, 470, 490, 510, 530, 550, 570, 590, 610, 630, 650, 670, 690, 710, 730, 750, 770, 790, or 810. In some instances, the antibody includes an FR-H3 comprising an amino acid sequence any one of SEQ ID NOS: 91, 111, 131, 151, 171, 191, 211, 231, 251, 271, 291, 311, 331, 351, 371, 391, 411, 431, 451, 471, 491, 511, 531, 551, 571, 591, 611, 631, 651, 671, 691, 711, 731, 751, 771, 791, or 811. In some instances, the antibody includes an FR-H4 comprising an amino acid sequence any one of SEQ ID NOs: 92, 112, 132, 152, 172, 192, 212, 232, 252, 272, 292, 312, 332, 352, 372, 392, 412, 432, 452, 472, 492, 512, 532, 552, 572, 592, 612, 632, 652, 672, 692, 712, 732, 752, 772, 792, or 812.

In some instances, the antibody includes an FR-L1 comprising an amino acid sequence any one of SEQ ID NOs: 93, 113, 133, 153, 173, 193, 213, 233, 253, 273, 293, 313, 333, 353, 373, 393, 413, 433, 453, 473, 493, 513, 533, 553, 573, 593, 613, 633, 653, 673, 693, 713, 733, 753, 773, 793, 813. In some instances, the antibody includes an FR-L2 comprising an amino acid sequence any one of SEQ ID NOS: 94, 114, 134, 154, 174, 194, 214, 234, 254, 274, 294, 314, 334, 354, 374, 394, 414, 434, 454, 474, 494, 514, 534, 554, 574, 594, 614, 634, 654, 674, 694, 714, 734, 754, 774, 794, or 814. In some instances, the antibody includes an FR-L3 comprising an amino acid sequence any one of SEQ ID NOs: 95, 115, 135, 155, 175, 195, 215, 235, 255, 275, 295, 315, 335, 355, 375, 395, 415, 435, 455, 475, 495, 515, 535, 555, 575, 595, 615, 635, 655, 675, 695, 715, 735, 755, 775, 795, or 815. In some instances, the antibody includes an FR-L4 comprising an amino acid sequence any one of SEQ ID NOs: 96, 116, 136, 156, 176, 196, 216, 236, 256, 276, 296, 316, 336, 356, 376, 396, 416, 436, 456, 476, 496, 516, 536, 556, 576, 596, 616, 636, 656, 676, 696, 716, 736, 756, 776, 796, or 816.

In some instances, the antibody includes a heavy chain comprising an amino acid sequence any one of SEQ ID NOs: 97, 117, 137, 157, 177, 197, 217, 237, 257, 277, 297, 317, 337, 357, 377, 397, 417, 437, 457, 477, 497, 517, 537, 557, 577, 597, 617, 637, 657, 677, 697, 717, 737, 757, 777, 797, or 817. In some instances, the antibody includes a heavy chain comprising a nucleic acid sequence any one of SEQ ID NOs: 99, 119, 139, 159, 179, 199, 219, 239, 259, 279, 299, 319, 339, 359, 379, 399, 419, 439, 459, 479, 499, 519, 539, 559, 579, 599, 619, 639, 659, 679, 699, 719, 739, 759, 779, 799, or 819. In some instances, the antibody includes a light chain comprising an amino acid sequence any one of SEQ ID NOs: 98, 118, 138, 158, 178, 198, 218, 238, 258, 278, 298, 318, 338, 358, 378, 398, 418, 438, 458, 478, 498, 518, 538, 558, 578, 598, 618, 638, 658, 678, 698, 718, 738, 758, 778, 798, or 818. In some instances, the antibody includes a light chain comprising a nucleic acid sequence any one of SEQ ID NOs: 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, or 820. In some instances, the antibody includes a heavy chain and a light chain sequence from any one of the following antibodies: V5-V41.

Anti-Ara h 2 antibodies disclosed herein may be modified according to any standard methods such as those described herein. Accordingly, such modified antibodies are also useful in the compositions and methods described herein.

Furthermore, the invention further features any isolated antibody that binds to Ara h 2 (conglutin-7), or an antigen-binding fragment thereof, wherein the antibody comprises its respective components as described herein, for example, where the antibody includes following six complementarity determining regions (CDRs):
(a) an CDR-H1 comprising its respective amino acid sequence of SEQ ID NOs: 81, 101, 121, 141, 161, 181, 201, 221, 241, 261, 281, 301, 321, 341, 361, 381, 401, 421, 441, 461, 481, 501, 521, 541, 561, 581, 601, 621, 641, 661, 681, 701, 721, 741, 761, 781, or 801;
(b) an CDR-H2 comprising its respective amino acid sequence of SEQ ID NOs: 82, 102, 122, 142, 162, 182, 202, 222, 242, 262, 282, 302, 322, 342, 362, 382, 402, 422, 442, 462, 482, 502, 522, 542, 562, 582, 602, 622, 642, 662, 682, 702, 722, 742, 762, 782, or 802;
(c) an CDR-H3 comprising its respective amino acid sequence of SEQ ID NOs: 83, 103, 123, 143, 163, 183, 203, 223, 243, 263, 283, 303, 323, 343, 363, 383, 403, 423, 443, 463, 483, 503, 523, 543, 563, 583, 603, 623, 643, 663, 683, 703, 723, 743, 763, 783, or 803;
(d) an CDR-L1 comprising its respective amino acid sequence of SEQ ID NOs: 84, 104, 124, 144, 164, 184, 204, 224, 244, 264, 284, 304, 324, 344, 364, 384, 404, 424, 444, 464, 484, 504, 524, 544, 564, 584, 604, 624, 644, 664, 684, 704, 724, 744, 764, 784, or 804;
(e) an CDR-L2 comprising its respective amino acid sequence of SEQ ID NOs: 85, 105, 125, 145, 165, 185, 205, 225, 245, 265, 285, 305, 325, 345, 365, 385, 405, 425, 445, 465, 485, 505, 525, 545, 565, 585, 605, 625, 645, 665, 685, 705, 725, 745, 765, 785, or 805; and
(f) an CDR-L3 comprising its respective amino acid sequence 86, 106, 126, 146, 166, 186, 206, 226, 246, 266, 286, 306, 326, 346, 366, 386, 406, 426, 446, 466, 486, 506, 526, 546, 566, 586, 606, 626, 646, 666, 686, 706, 726, 746, 766, 786, or 806.

Antibodies of the invention may, for example, be monoclonal, human, humanized, or chimeric. The antibodies can be full-length antibodies or antibody fragments thereof (e.g., an antibody fragment that binds Ara h 2). The antibody fragment may be selected from the group consisting of Fab, Fab′-SH, Fv, scFv, and (Fab′)₂ fragments. In some instances, the antibody is an IgG antibody (e.g., an IgG1 antibody). An antibody of the invention may have a half-life of ≥3 days (e.g., ≥1 week, e.g., ≥2 weeks, e.g., ≥1 month, e.g., ≥2 months, e.g., ≥3 months, e.g., ≥4 months, e.g., ≥5 months, e.g., ≥6 months).

In a further aspect, an anti-Ara h 2 antibody according to any of the above embodiments may incorporate any of the features, singly or in combination, as described in Sections 1-5 below.

### 1. Antibody Affinity

In certain embodiments, an antibody provided herein may have a dissociation constant (K_{D}) of ≤10 μM, ≤1 μM, ≤100 nM, ≤10 nM, ≤1 nM, ≤0.1 nM, or ≤0.01 nM.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-3000 (BIAcore, Inc., Piscataway, NJ) is performed at 25° C. with immobilized antigen CM5 chips at ~10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (~0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio kₒₙ/k_{off}. See, for example, Chen et al., J. Mol. Biol. 293:865-881 (1999). If the on-rate exceeds 106M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, which are known in the art. Also included are diabodies, which have two antigen-binding sites that may be bivalent or bispecific, as is known in the art. Triabodies and tetrabodies are also known. Single-domain antibodies are also antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody (e.g., a human monoclonal antibody (HuMab), e.g., an anti-Ara h 2 HuMab). Human antibodies can be produced using various techniques known in the art.

In some instances, human antibodies are obtained by cloning the heavy and light chain genes directly from human B cells obtained from a human subject as is described herein in Example 1. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen (e.g., Ara h 2).

In some instances, human antibodies may be prepared by administering an immunogen (e.g., Ara h 2) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, human antibodies can also be made by hybridoma-based methods, as described in further detail below. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Antibody Variants

In certain embodiments, amino acid sequence variants of the anti-Ara h 2 antibodies are contemplated.

For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process, and/or residues that contact antigen, with the resulting variant VH or VL being tested for binding affinity. Affinity maturation by constructing and reselecting from secondary libraries is known in the art. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, alternations may be made to the Fc region of an antibody. These alterations can be made alone, or in addition to, alterations to one or more of the antibody variable domains (i.e., VH or VL regions) or regions thereof (e.g., one or more CDRs or FRs). The alterations to the Fc region may result in reduced antibody effector functions (e.g., complement-dependent cytotoxicity (CDC))

In certain instances, the invention contemplates an antibody, e.g., antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

In certain embodiments, alterations of the amino acid sequences of the Fc region of the antibody may alter the half-life of the antibody in the host. Certain mutations that alter binding to the neonatal Fc receptor (FcRn) may extend half-life of antibodies in serum. For example, antibodies that have tyrosine in heavy chain position 252, threonine in position 254, and glutamic acid in position 256 of the heavy chain can have dramatically extended half-life in serum (see, e.g., U.S. Pat. No. 7,083,784).

In other embodiments, Fc modifications are introduced to maximize dosing levels and to prevent anaphylaxis of peanut allergy.

In certain instances, antibodies of the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In certain instances, it is desirable to create cysteine engineered anti-Ara h 2 antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

In certain instances, an antibody of the invention provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Isolation and Characterization of Human Antibodies to Ara h 2

An exemplary method for identifying anti-Ara h 2 antibodies is described in Example 1.

Sequence information for antibodies described herein can be ascertained using sequencing techniques which are well known in the art.

Similarly, affinity of the antibodies for anti-Ara h 2 antibodies can also be assessed using standard techniques. For example, Biacore 3000 can be used to determine the affinity of such antibodies. Antibodies are captured on the surface of a Biacore chip (GE healthcare), for example, via amine coupling (Sensor Chip CM5). The captured antibodies can be exposed to various concentrations of Ara h 2 in solution, and the Kₒₙ and K_{off} for an affinity (K_{D}) can be calculated, for example, by BIAevaluation software.

Antibodies can also be characterized for binding to Ara h 2 using a variety of known techniques, such as ELISA, Western blot, etc. Generally, the antibodies are initially characterized by ELISA. Briefly, microtiter plates can be coated with purified Ara h 2 in PBS, and then blocked with irrelevant proteins such as bovine serum albumin (BSA) diluted in PBS. Dilutions of plasma are added to each well and incubated for 1-2 hours at 37° C. The plates are washed with PBS/Tween 20 and then incubated with a goat-anti-human IgG Fc-specific polyclonal reagent conjugated to alkaline phosphatase for 1 hour at 37° C. After washing, the plates are developed with ABTS substrate, and analyzed at OD of 405.

In other instances, competition assays may be used to identify an antibody that competes with an anti-Ara h 2 antibody for binding to Ara h 2. In certain embodiments, such a competing antibody binds to the same epitope (e.g., a linear or a conformational epitope) that is bound by an anti-Ara h 2 antibody of the invention. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized Ara h 2 is incubated in a solution comprising a first labeled antibody that binds to Ara h 2 and a second unlabeled antibody that is being tested for its ability to compete with the first antibody for binding to Ara h 2. The second antibody may be present in a hybridoma supernatant. As a control, immobilized Ara h 2 is incubated in a solution comprising the first labeled antibody but not the second unlabeled antibody. After incubation under conditions permissive for binding of the first antibody to Ara h 2, excess unbound antibody is removed, and the amount of label associated with immobilized Ara h 2 is measured. If the amount of label associated with immobilized Ara h 2 is substantially reduced in the test sample relative to the control sample, then that indicates that the second antibody is competing with the first antibody for binding to Ara h 2.

### C. Compositions

In another aspect, the invention features a pharmaceutical composition comprising a therapeutically effective amount of one or more isolated human antibodies or antigen-binding fragments thereof that specifically bind Ara h 2, together with one or more pharmaceutically acceptable excipients. In one embodiment, the pharmaceutical composition comprises a therapeutically effective amount of two or more isolated human antibodies or antigen-binding fragments thereof that specifically bind Ara h 2 together with one or more pharmaceutically acceptable excipients.

In one embodiment, the invention features a composition, which is a combination of a therapeutically effective amount of one or more anti-Ara h 2 antibodies or antigen-binding fragments thereof of the invention, and a therapeutically effective amount of a second therapeutic agent. The second therapeutic agent may be a small molecule drug, a protein/polypeptide, an antibody, a nucleic acid molecule, such as an anti-sense molecule, or a siRNA. The second therapeutic agent may be synthetic or naturally derived.

The second therapeutic agent may be any agent that is advantageously combined with an antibody or fragment thereof of the invention, for example, a second antibody other than those described herein that is capable of blocking the binding of Ara h 2 to IgE present on mast cells or basophils. A second therapeutic agent may also be any agent that is used as standard of care in treating an allergic response to any allergen. Such second therapeutic agent may be an antihistamine, epinephrine, a decongestant, or a corticosteroid.
In certain embodiments, the second therapeutic agent may be an agent that helps to counteract or reduce any possible side effect(s) associated with the antibody or antigen-binding fragment of an antibody of the invention, if such side effect(s) should occur.
It will also be appreciated that the antibodies and pharmaceutically acceptable compositions of the present invention can be employed in combination therapies, that is, the antibodies and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an antibody may be administered concurrently with another agent used to treat the same disorder), or they may achieve different effects (e.g., control of any adverse effects). As used herein, additional therapeutic agents that are normally administered to treat or prevent a particular disease, or condition, are appropriate for the disease, or condition, being treated.
When multiple therapeutics are co-administered, dosages may be adjusted accordingly, as is recognized in the pertinent art.

Accordingly, the present invention provides a composition, e.g., a pharmaceutical composition, containing one or more (e.g., 1, 2, 3, or 4 or more) of the anti-Ara h 2 antibodies, or antibody fragments thereof, disclosed herein (e.g., antibodies V1-V41). The antibodies, if desired, may be modified according to any of the modifications outlined above. The pharmaceutical compositions may be formulated together with a pharmaceutically acceptable carrier, excipient, or diluent. In some instances, the pharmaceutical compositions include two or more of the anti-Ara h 2 antibodies. Preferably, each of the antibodies of the composition binds to a distinct epitope of Ara h 2.

A pharmaceutical composition described herein can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Active ingredients of the pharmaceutical composition may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, such as TWEEN® 80. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, genes encoding the anti-Ara h 2 antibodies may be delivered directly into the subject for expression rather than administering purified antibodies for prevention or therapy. For example, viral vectors, such as recombinant viruses, can be used to deliver the heavy and light chain genes. In one example, rAAV virus particles can be used to deliver anti-HIV monoclonal antibodies (Balazs et al. Nature. 481:81, 2012). Antibody genes could also be effectively delivered by electroporation of muscle cells with plasmid DNA containing heavy and/or light chain genes (e.g., VH and/or VL genes) (Muthumani et al. Hum Vaccin Immunother. 10:2253, 2013). Lentivirus vectors or other nucleic acids (e.g., RNA) capable of delivering transgenes could also be used to delivery antibody genes to establish serum antibody levels capable of prevention.

Also contemplated are kits including human anti-Ara h 2 antibodies and, optionally, instructions for use. The kits can further contain one or more additional reagents, such as a second, different anti-Ara h 2 antibody having a complementary activity that binds to an epitope on Ara h 2 that is distinct from the epitope to which the first anti-Ara h 2 antibody binds.

### D. Therapy

Any of the anti-Ara h 2 antibodies described herein and compositions containing the antibodies can be used in a variety of in vitro and in vivo therapeutic applications. In some embodiments, an anti-Ara h 2 antibody may be used as a monotherapy. In some embodiments, an anti-Ara h 2 antibody may be used as a combination therapy.

The invention provides an anti-Ara h 2 antibody for use as a medicament. In further aspects, an anti-Ara h 2 antibody for use in treating a peanut allergy is provided. In certain embodiments, an anti-Ara h 2 antibody for use in a method of treatment is provided. In certain embodiments, the invention provides an anti-Ara h 2 antibody for use in a method of treating an individual having a peanut allergy comprising administering to the individual an effective amount of the anti-Ara h 2 antibody. In one such embodiment, the method further comprises administering to the individual an effective amount of at least one additional therapeutic agent, for example, as described below.

The invention provides an anti-Ara h 2 antibody in the manufacture or preparation of a medicament. In a further aspect, the invention provides for the use of an anti-Ara h 2 antibody in the manufacture or preparation of a medicament. In one embodiment, the medicament is for treatment of a peanut allergy. In a further embodiment, the medicament is for use in a method of treating a peanut allergy, e.g., comprising administering to an individual having a peanut allergy an effective amount of the medicament. In one such embodiment, the method further comprises administering to the individual an effective amount of at least one additional therapeutic agent, e.g., as described below.

In a further aspect, the invention provides a method for treating a peanut allergy. In some instances, the method comprises administering the individual having such a peanut allergy an effective amount of an anti-Ara h 2 antibody. In one embodiment, the method comprises administering to an individual having such peanut allergy an effective amount of an anti-Ara h 2 antibody (e.g., any anti-Ara h 2 antibody disclosed herein). In one such embodiment, the method further comprises administering to the individual an effective amount of at least one additional therapeutic agent, as described below.

In a further aspect, the invention provides pharmaceutical formulations comprising any of the anti-Ara h 2 antibodies provided herein, e.g., for use in any of the above therapeutic methods. In one embodiment, a pharmaceutical formulation comprises any of the anti-Ara h 2 antibodies provided herein and a pharmaceutically acceptable carrier.

In a further aspect, the invention features a method of treating a subject having a peanut allergy or who is sensitized to peanuts comprising administering a therapeutically effective amount of an antibody (e.g., a human monoclonal antibody) that specifically binds to Ara h 2 or a pharmaceutical composition thereof, thereby treating the subject.

Accordingly, the invention features method for treating a patient who demonstrates a sensitivity to a peanut allergen (e.g., Ara h 2), an allergic reaction against a peanut allergen, or to Ara h 2 protein, or for treating at least one symptom or complication associated with a sensitivity to, or allergic reaction against a peanut allergen, an allergic reaction against a peanut allergen, or to Ara h 2 protein, comprising administering an effective amount of one or more isolated human monoclonal antibodies or antigen-binding fragments thereof that bind specifically to Ara h 2, according to claims describe herein for T1 (also referred to as V1), T3 (also referred to as V2), T4 (also referred to as V3), or T5 (also referred to as V4) to a patient in need thereof, wherein the sensitivity to, or an allergic reaction against, to a peanut allergen, an allergic reaction against a peanut allergen, or to Ara h 2 protein, is lessened in severity and/or duration, or at least one symptom or complication associated with the sensitivity to, or allergic reaction against, to a peanut allergen, an allergic reaction against a peanut allergen, or to Ara h 2 protein is ameliorated, or that the frequency and/or duration of, or the severity of the sensitivity to or allergic reaction against, to a peanut allergen, an allergic reaction against a peanut allergen, or to Ara h 2 protein, is reduced following administration of one or more of the isolated human monoclonal antibodies or fragments thereof that bind specifically to Ara h 2.

Preferably, the method of treatment further comprises administering an effective amount of a second therapeutic agent useful for diminishing an allergic reaction to a peanut allergen, an allergic reaction against a peanut allergen, or to Ara h 2 protein. In some instances, the second therapeutic agent is selected from the group consisting of a corticosteroid, a bronchial dilator, an antihistamine, epinephrine, or a decongestant. Typically, treatment results in a reduction in allergic rhinitis, allergic conjunctivitis, allergic asthma, or an anaphylactic response following exposure, direct or indirect, of the patient to a peanut allergen, an allergic reaction against a peanut allergen, or to Ara h 2 protein.

Furthermore, as is discussed herein, antibodies of the invention can be used either alone or in combination with other agents in a therapy. For instance, an antibody of the invention may be co-administered with at least one additional therapeutic agent (e.g., a corticosteroid, a bronchial dilator, an antihistamine, epinephrine, and/or a decongestant). Such combination therapies encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-Ara h 2 antibody and administration of an additional therapeutic agent occur within about one month, or within about one, two or three weeks, or within about one, two, three, four, five, or six days, of each other.

Anti-Ara h 2 antibodies described herein may also be used in combination.

An antibody, such as modified antibodies disclosed herein such as T1 (also referred to as V1), T3 (also referred to as V2), T4 (also referred to as V3), T5 (also referred to as V4), or V5-V41 (and/or any additional therapeutic agent) can be administered by any suitable means, including parenteral, intrapulmonary, intranasal, oral, mucosal, intravenous, and, if desired for local treatment, intralesional administration. Parenteral infusions include intramuscular, intravenous, intraarterial, intraperitoneal, or subcutaneous administration. In some instances, an anti-Ara h 2 antibody (e.g., any anti-Ara h 2 antibody disclosed herein) may be administered orally, intrarectally, mucosally, intravenously, intramuscularly, intradermally, transdermally, subcutaneously, percutaneously, intraarterially, intraperitoneally, intravitreally, topically, intralesionally, intraarticularly, intraprostatically, intrapleurally, intratracheally, intrathecally, intranasally, intravaginally, intratumorally, intraperitoneally, peritoneally, intraventricularly, intracranially, subconjunctivally, intravesicularly, intrapericardially, intraumbilically, intraorbitally, ocularly, intraocularly, juxtasclerally, subtenonly, superchoroidally, by inhalation, by injection, by eye drop, by implantation, by infusion, by continuous infusion, by localized perfusion bathing target cells directly, by catheter, by lavage, in cremes, or in lipid compositions. In certain instances, antibody genes (e.g., genes encoding any one or more of the anti-Ara h 2 antibodies of the invention could be administered as a gene therapy to produce the one or more anti-Ara h 2 antibodies in the subject using either DNA vectors or viral vectors (e.g., rAAV vectors). Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a peanut allergy or a subject who is hypersensitive to peanuts, the appropriate dosage of an antibody of the invention (when used alone or in combination with one or more other additional therapeutic agents) will depend on the severity of the allergic reaction to be prevented/treated, the duration of effective antibody concentration required, the type of antibody, the severity and course of the disease, whether the antibody is administered for preventive or therapeutic purposes, previous therapy, the patient's clinical history and response to the antibody, and the discretion of the attending allergist. The antibody is suitably administered to the patient at one time or over a series of treatments. For repeated administrations over several days or longer, depending on the condition, the treatment would generally be sustained until a desired suppression of disease symptoms occurs. Doses may be administered intermittently, e.g. every week, every two weeks, every three weeks, every four weeks, every five weeks, every six weeks, every seven weeks, every eight weeks, every nine weeks, every ten weeks, every eleven weeks, or every twelve weeks (e.g., such that the patient receives from about two to about twenty, or e.g., about six doses of the antibody). For example, a dose may be administered once per month, once every two months, or once every three months (e.g., by subcutaneous injection) as an initial higher loading dose, followed by one or more lower doses may be administered. However, other dosage regimens may be useful. The progress of this therapy is easily monitored by conventional techniques and assays.

Actual dosage levels of the active ingredients in the pharmaceutical compositions of the present invention may be varied so as to obtain an amount of the active ingredient which is effective to achieve the desired therapeutic response and duration for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. An allergist having ordinary skill in the art can readily determine and prescribe the effective amount of the pharmaceutical composition required. For example, the allergist can start doses of the compounds of the invention employed in the pharmaceutical composition at levels lower than that required in order to achieve the desired therapeutic effect and gradually increase the dosage until the desired effect is achieved. In general, a suitable daily dose of compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In some instances, the antibody-based therapy may be combined with an additional therapy for more efficacious treatment (e.g., additive or synergistic treatment) of the subject. Accordingly, subjects treated with antibodies of the invention can be additionally administered (prior to, simultaneously with, or following administration of a human antibody of the invention) with another therapeutic agent which enhances or augments the therapeutic effect of the human antibodies.

### E. Diagnostics

In certain embodiments, any of the anti-Ara h 2 antibodies of the invention are useful for in vitro or in vivo detection of peanut tolerance in allergic individuals. The term “detecting” as used herein encompasses quantitative or qualitative detection. In certain embodiments, a biological sample comprises a cell or tissue.

Current data suggests that there are three dominant conformational epitope bins where IgE binds to the Ara h 2 antigen and the recombinant IgG neutralizing anti Ara h 2 antibodies will compete for these sites and inhibit or displace IgE binding. With these neutralizing antibodies, the current tandem epitope binning by biolayer light interferometry assays can be used to assay whether a sample (e.g. a serum sample from a patient believed to have been exposed to a peanut allergen or a patient undergoing an OIT) contains antibodies that bind the same epitope bin, and whether those antibodies can effectively compete with the neutralizing antibodies. This test is useful to track the progress of therapy or to predict whether a patient will obtain the outcome of sustained unresponsiveness after OIT by assaying that patient's early OIT serum sample.

Accordingly, prior to the initiation of OIT, IgG antibodies isolated from patient serum will lack significant Ara h 2 neutralizing capacity and will be unable to compete with antibodies disclosed herein. During the course of treatment, patients that are responding to OIT will begin to produce IgG antibodies that are capable of competing with the anti-Ara h 2 antibodies while patients that are not responding to the therapy will maintain low levels of Ara h 2 neutralizing antibodies. The results of the competition assay can be used to predict whether an individual patient will attain a desired outcome of the treatment or can be used to adjust the duration of treatment to improve the likelihood of a desired outcome.

### F. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. In some embodiments, the article of manufacture comprises an additional therapeutic agent (e.g., a corticosteroid, a bronchial dilator, an antihistamine, epinephrine, and/or a decongestant).

Other embodiments of the present invention are described in the following Examples. The present invention is further illustrated by the following examples which should not be construed as further limiting. The contents of all references, patents and published patent applications cited throughout this application are expressly incorporated herein by reference.

### III. Examples

### Example 1—Isolation of Genes Encoding Anti-Ara h 2 Antibodies from Patients Having Sustained Unresponsiveness to Peanut Allergen after One Month of Peanut Avoidance

The peanut-specific protective antibodies directed towards the immunodominant peanut antigen Ara h 2 were cloned from the circulation of pediatric patients who had sustained tolerance to peanut after OIT. From the same peanut OIT study, suppression of basophil activation was identified as a good biomarker of future tolerance. Furthermore, the suppression of basophils is mediated by IgG in the sera of tolerant patients, from whom Ara h 2-specific antibodies were cloned. The protective antibodies were identified as those able to outcompete non-tolerant antibodies using biolayer light interferometry, as those have better antigen blocking capabilities.

### Patient Selection

Peripheral blood was obtained after IRB-approved consent from peanut allergic participants of an open-label randomized trial of peanut OIT (NCT01324401). Study inclusion criteria included participants aged 7-21 years old with a diagnosis of peanut allergy based on a clinical history of reaction to peanut within one hour of ingestion and either an elevated skin prick test (>8 mm wheal) or elevated peanut-specific IgE (CAP FEIA >10 kU/L).

A total of 30 patients were enrolled, including 4 in an observational control arm, who were monitored for 1 year, after which they also received active OIT with premeasured peanut flour (Medium Roast, Golden Peanut Co., Alpharetta, GA). The protocol began with a 1 day modified rush before the build up phase to a 12 week maintenance phase with 4 grams of peanut protein, at the end of which desensitization was evaluated with an oral food challenge. After 4 weeks of peanut avoidance, subjects underwent a double blind oral food challenge to peanut.

Of the 22 subjects who were effectively desensitized, 9 subjects continued to have sustained unresponsiveness (SU) after a month of peanut avoidance and 13 subjects were found to have only transient desensitization (TD).

### Specific Immunoglobulin E Measurement

Measurement of antigen-specific immunoglobulin levels in the serum or plasma from peripheral blood of subjects undergoing OIT was determined using the ImmunoCAP 1000 instrument (Phadia AB) according to the manufacturer's instructions.

### Ara h 2 Specific B Cells Identification

Ara h 2 specific B cells from peripheral blood were identified by flow cytometry using a fluorescent tetramer created as described by Patil et al., J. Allergy Clin Immunol. 136(1):125-134 (2015). Briefly, peripheral blood mononuclear cells (PBMCs) isolated by density gradient centrifugation (Ficoll-Paque Plus; GE Healthcare) from peripheral blood of patients undergoing peanut OIT were then stained using CD3-APC (eBioscience clone OKT3), CD14-APC (eBioscience, clone 61D3), CD16-APC (eBioscience clone CB16), CD19-APC-Cy7 (BD Biosciences clone SJ25C1), CD27-PE (BD Pharmingen clone M-T271), CD38-Violet 421 (BD Biosciences clone HIT2), IgM-PE-Cy5 (BD Pharmingen clone G20-127), and AF488-Ara h 2 multimer. Data were analyzed using FlowJo 8.8.7 software (TreeStar).

Ara h 2 specific B cells, identified as tetramer⁺CD19⁺ cells were isolated 1-2 months after the start of OIT for single-cell indexed fluorescence-activated cell sorting (BD Aria II, BD Fusion SORP) into 96-well plates (Eppendorf) for further single cell nested RT-PCR BCR amplification. Briefly, cells sorted into RNAsin containing single strand buffer were frozen at −80 C and subsequently underwent heat lysis with NP-40 and random hexamers. The RT reaction was followed by 2 nested PCR reactions for both heavy and light chains, as described by Patil et al., J. Allergy Clin Immunol. 136(1):125-134 (2015).

Successfully amplified products were Sanger sequenced (Genewiz Inc.) using second PCR primers. Consensus sequences combining both the forward and reverse sequences were determined using pairwise alignment in Geneious (Biomatters Ltd). These sequences were then analyzed using IMGT/V-BLAST to identify germline V, D, and J sequences with the highest identity, CDR3 regions, and nucleotide and amino acid changes from germline sequence.

Purified (Qiagen PCR purification kit) paired heavy and light chains subsequently underwent a third PCR reaction for the addition of restriction enzyme sites, followed by repeat PCR purification. For expression as recombinant antibodies on an IgG₁ backbone, digested heavy and light PCR amplicons were ligated into heavy and light chain vectors, respectively, as described by Patil et al., J. Allergy Clin Immunol. 136(1):125-134 (2015), and transformed in competent E. coli NEB5α bacteria (New England Biolabs) for ampicillin selection of plasmids with Sanger-sequence verified PCR amplicons. Plasmid DNA (25 ng) from selected heavy and light chains were transfected into HEK293 T cells using GenJet™ In Vitro DNA Transfection Reagent (SignaGen). Supernatants were harvested from cells after three days of culture at 37° C. with 5% CO2 in serum free HL-1 media (Lonza) supplemented with Pen-Strep and 8 mM Glutamax (Gibco). Antibodies were purified from supernatants using Protein G beads (ThermoFischer).

Recombinant Ara h 2 antibody specificity was verified by ImmunoCAP measurement for each antibody. Epitope binning was performed using SA-sensors (ForteBio) loaded with 0.5 μM concentration of biotinylated Ara h 2 (Indoor Technologies) by biolayer light interferometry (ForteBio Octet Red96). Epitope binning was performed using Octet Data Analysis Software.

In order to be expressed as monoclonal antibodies, restriction sites were added during cloning through the use of highly permissive, degenerate primers situated in the FR1 and FR4 regions, for subsequent cloning into a vector with an IgG1 constant region. As a result of both PCR error introduction rates as well as the primers required to add the requisite restriction enzyme cleavage sites, the original VDJ sequence cloned from RNA, rather than DNA, is now altered. The altered proteins are expressed as recombinant antibodies for further characterization. Furthermore, the original antibodies may have been an isotype other than IgG1 but are then artificially made into IgG1 (or any other) constant region containing antibody.

### Example 2—Antibody Binding to Ara h 2 as Determined by Surface Plasmon Resonance

Calculated equilibrium dissociative constants (K_{D}) for antigen binding to anti-Ara h 2 antibodies was determined using a real-time surface plasmon resonance biosensor (Biacore 3000) assay. Exemplary results are shown in Table 2.

TABLE 2

| ab | KD |
|---|---|
| T1 | 4.85E−09 |
| T3 | 1.93E−10 |
| T4 | 1.43E−12 |
| T5 | 1.12E−10 |

### Example 3—Anti-Ara h 2 Antibody Epitope Characterization

The epitopes of antibodies T1 (also referred to as V1), T3 (also referred to as V2), T4 (also referred to as V3), and T5 (also referred to as V4) were characterized using biotinylated Ara h 2 by epitope binning by biolayer light interferometry (ForteBio, Octet). Experiments showed that antibodies T1 and T3 share the same epitope bin (Bin 1) and antibodies T4 and T5 share a different epitope bin (Bin 2).

### Other Embodiments

Some embodiments of the technology described herein can be defined according to any of the following numbered embodiments:

### T1:

1. An isolated antibody that binds to Ara h 2 (conglutin-7), or an antigen-binding fragment thereof, wherein the antibody comprises the following six complementarity determining regions (CDRs):
(a) an CDR-H1 comprising the amino acid sequence of GFTFRDYS (SEQ ID NO: 1);
(b) an CDR-H2 comprising the amino acid sequence of IRFDGTTK (SEQ ID NO: 2);
(c) an CDR-H3 comprising the amino acid sequence of VKDNGWRAFDH (SEQ ID NO: 3);
(d) an CDR-L1 comprising the amino acid sequence of QSLLHRNGYIY (SEQ ID NO: 4);
(e) an CDR-L2 comprising the amino acid sequence of FVS (SEQ ID NO: 5); and
(f) an CDR-L3 comprising the amino acid sequence of MQSLENPWT (SEQ ID NO: 6).
   2. The antibody of embodiment 1, wherein the antibody comprises
(a) a heavy chain variable (VH) domain comprising an amino sequence having at least 90%, at least 95%, or at least 99% sequence identity to the amino acid sequence of SEQ ID NO: 7;
(b) a light chain variable (VL) domain comprising an amino acid sequence having at least 90%, at least 95%, or at least 99% identity to the amino acid sequence of SEQ ID NO: 8; or (c) a VH domain as in (a) and a VL domain as in (b).
   3. The antibody of embodiment 1 or 2, further comprising the following VH domain framework regions (FRs):
(a) an FR-H1 comprising the amino acid sequence of VESGGGVVQPGASLRLSCVAS (SEQ ID NO: 9);
(b) an FR-H2 comprising the amino acid sequence of MHWVRQAPGKGLEWMSF (SEQ ID NO: 10);
(c) an FR-H3 comprising the amino acid sequence of DYRESVKGRFIISRDDSKNTLYLQMTSLRPEDTARYYC (SEQ ID NO: 11); and
(d) an FR-H4 comprising the amino acid sequence of WGQGALVIVSS (SEQ ID NO: 12).
   4. The antibody of any one of embodiments 1-3, wherein the VH domain comprises the amino acid sequence of SEQ ID NO: 7.
   5. The antibody of any one of embodiments 1-4, further comprising the following VL domain FRs:
(a) an FR-L1 comprising the amino acid sequence of EIVMTQSPLSLPVTPGEAASISCRSS (SEQ ID NO: 13);
(b) an FR-L2 comprising the amino acid sequence of LDWYLQRPGQSPQLLIS (SEQ ID NO: 14);
(c) an FR-L3 comprising the amino acid sequence of KRASGAPDRFSGSGSGTDFTLTISRVEAEDFGVYFC (SEQ ID NO: 15); and
(d) an FR-L4 comprising the amino acid sequence of FGP (SEQ ID NO: 16).
   6. The antibody of any one of embodiments 1-5, wherein the VL domain comprises the amino acid sequence of SEQ ID NO: 8.
   7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 7 and
(b) a VL domain comprising an amino acid sequence having at least 90%, at least 95%, or at least 99% sequence identity to the amino acid sequence of SEQ ID NO: 8.
   8. The antibody of embodiment 7, wherein the antibody comprises a VH domain comprising the amino acid sequence of SEQ ID NO: 7 and a VL domain comprising the amino acid sequence of SEQ ID NO: 8.
   9. The antibody of any one of embodiments 1-8, wherein the antibody comprises (a) a heavy chain comprising the amino acid sequence of SEQ ID NO: 17 and (b) a light chain comprising the amino acid sequence of SEQ ID NO: 18.
   10. The antibody of any one of embodiments 1-9, wherein the antibody binds Ara h 2 with a K_{D} of about 50 nM or less.
   11. The method of embodiment 10, wherein the K_{D} is measured by a surface plasmon resonance assay.
   12. The antibody of embodiment 11, wherein the antibody binds Ara h 2 with a K_{D} of between about 0.1 pM and about 40 nM.
   13. The antibody of embodiment 12, wherein the antibody binds Ara h 2 with a K_{D} of between about 120 pM and about 20 nM.
   14. The antibody of embodiment 13, wherein the antibody binds Ara h 2 with a K_{D} of between about 120 pM and about 10 nM.
   15. The antibody of any one of embodiments 1-14, wherein the antibody is capable neutralizing Ara h 2
   16. An antibody that binds to the same epitope as the antibody of any one of embodiment 1-15.
   17. An antibody that competes for binding to Ara h 2 with the antibody of any one of embodiments 1-16.
   18. The antibody of embodiment 16 or 17, wherein whether the antibody binds to the same epitope or competes for binding to Ara h 2 is determined by an epitope binning assay.
   19. The antibody of any one of embodiments 1-18, wherein the antibody is monoclonal.
   20. The antibody of any one of embodiments 1-19, wherein the antibody is humanized.
   21. The antibody of any one of embodiments 1-20, wherein the antibody is an antibody fragment that binds Ara h 2.
   22. The antibody of embodiment 21, wherein the antibody fragment is selected from the group consisting of Fab, Fab′-SH, Fv, scFv, and (Fab′)₂ fragments.
   23. The antibody of any one of embodiments 1-20, wherein the antibody is a full-length antibody.
   24. The antibody of embodiment 23, wherein the antibody is an IgG antibody.
   25. The antibody of embodiment 24, wherein the IgG antibody is an IgG1 antibody.
   26. The antibody of embodiment 24, wherein the IgG antibody is an IgG4 antibody.
   27. The antibody of embodiment 26, wherein the IgG4 antibody comprises an S228P mutation (EU numbering).
   28. The antibody of any one of embodiments 1-27, wherein the antibody is a monospecific antibody.
   29. The antibody of any one of embodiments 1-27, wherein the antibody is a multispecific antibody.
   30. The antibody of embodiment 29, wherein the antibody is a bispecific antibody.
   31. A polynucleotide encoding an isolated antibody of any one of embodiments 1-30.
   32. A vector comprising the polynucleotide of embodiment 31.
   33. A host cell comprising the vector of embodiment 32.
   34. A method of producing the antibody of any one of embodiments 1-33, the method comprising culturing a host cell that comprises the vector of embodiment 32 and recovering the antibody.
   35. The method of embodiment 34, wherein the host cell is prokaryotic.
   36. The method of embodiment 35, wherein the host cell is Escherichia coli.
   37. The method of embodiment 34, wherein the host cell is eukaryotic.
   38. The method of embodiment 37, wherein the host cell is a 293 cell, a CHO cell, a yeast cell, or a plant cell.
   39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of embodiments 1-30.
   40. The method of embodiment 39, further comprising administering an effective amount of a second therapeutic agent useful for diminishing an allergic reaction an allergic reaction against, a nut or a portion thereof or an extract thereof, or to Ara h 2 protein.
   41. A method for detecting the presence of Ara h 2 neutralizing antibodies in a biological sample, comprising:
(a) providing a sample from a subject;
(b) contacting the sample with a mixture comprising:
   (i) the antibody of any one of embodiments 1-30 and
   (ii) Ara h 2, or a fragment thereof; and
(c) detecting the binding of antibodies present in the biological sample to Ara h 2, or a fragment thereof.
   42. The method of embodiment 41, where antibody binding is detected using tandem biolayer interferometry.
   43. The method of embodiments 41 or 42, where the antibody from step (b) is bound to a solid support.
   44. The method of embodiments 41 or 42, where Ara h 2, or a fragment thereof, is bound to a solid support.
   45. The method of embodiment 41, where antibody binding is detected using an enzyme-linked immunosorbent assay.

### T3:

1. An isolated antibody that binds to Ara h 2 (conglutin-7), or an antigen-binding fragment thereof, wherein the antibody comprises the following six CDRs:
(a) an CDR-H1 comprising the amino acid sequence of GFTFRDYS (SEQ ID NO: 21);
(b) an CDR-H2 comprising the amino acid sequence of IRFDGTTK (SEQ ID NO: 22);
(c) an CDR-H3 comprising the amino acid sequence of AKDNGWRAFDH (SEQ ID NO: 23);
(d) an CDR-L1 comprising the amino acid sequence of QSLLHRNGYIY (SEQ ID NO: 24);
(e) an CDR-L2 comprising the amino acid sequence of FVS (SEQ ID NO: 25); and
(f) an CDR-L3 comprising the amino acid sequence of MQALETPWT (SEQ ID NO: 26).
   2. The antibody of embodiment 1, wherein the antibody comprises
(a) a VH domain comprising an amino sequence having at least 90%, at least 95%, or at least 99% sequence identity to the amino acid sequence of SEQ ID NO: 27;
(b) a VL domain comprising an amino acid sequence having at least 90%, at least 95%, or at least 99% identity to the amino acid sequence of SEQ ID NO: 28; or (c) a VH domain as in (a) and a VL domain as in (b).
   3. The antibody of embodiment 1 or 2, further comprising the following VH domain FRs:
(a) an FR-H1 comprising the amino acid sequence of GGGVVQPGTSLRLSCVAS (SEQ ID NO: 29);
(b) an FR-H2 comprising the amino acid sequence of MHWVRQAPGKGLEWVSF (SEQ ID NO: 30);
(c) an FR-H3 comprising the amino acid sequence of DYRDSVKGRFIISRDDSKNTLYLQMTSLRVEDTALYYC (SEQ ID NO: 31); and
(d) an FR-H4 comprising the amino acid sequence of WGQGALVTVSS (SEQ ID NO: 32).
   4. The antibody of any one of embodiments 1-3, wherein the VH domain comprises the amino acid sequence of SEQ ID NO: 27.
   5. The antibody of any one of embodiments 1-4, further comprising the following VL domain FRs:
(a) an FR-L1 comprising the amino acid sequence of VMTQSPLSLPVTPGEAASISCRSS (SEQ ID NO: 33);
(b) an FR-L2 comprising the amino acid sequence of LDWYLQRPGQSPQLLIS (SEQ ID NO: 34);
(c) an FR-L3 comprising the amino acid sequence of KRASGAPDRFSGSGSGTDFTLTISRVEAEDFGVYFC (SEQ ID NO: 35); and
(d) an FR-L4 comprising the amino acid sequence of FGPGTKLEIK (SEQ ID NO: 36).
   6. The antibody of any one of embodiments 1-5, wherein the VL domain comprises the amino acid sequence of SEQ ID NO: 28.
   7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 27 and
(b) a VL domain comprising an amino acid sequence having at least 90%, at least 95%, or at least 99% sequence identity to the amino acid sequence of SEQ ID NO: 28.
   8. The antibody of embodiment 7, wherein the antibody comprises a VH domain comprising the amino acid sequence of SEQ ID NO: 27 and a VL domain comprising the amino acid sequence of SEQ ID NO: 28.
   9. The antibody of any one of embodiments 1-8, wherein the antibody comprises (a) a heavy chain comprising the amino acid sequence of SEQ ID NO: 37 and (b) a light chain comprising the amino acid sequence of SEQ ID NO: 38.
   10. The antibody of any one of embodiments 1-9, wherein the antibody binds Ara h 2 with a KD of about 50 nM or less.
   11. The method of embodiment 10, wherein the KD is measured by a surface plasmon resonance assay.
   12. The antibody of embodiment 11, wherein the antibody binds Ara h 2 with a KD of between about 0.1 pM and about 40 nM.
   13. The antibody of embodiment 12, wherein the antibody binds Ara h 2 with a KD of between about 120 pM and about 20 nM.
   14. The antibody of embodiment 13, wherein the antibody binds Ara h 2 with a KD of between about 120 pM and about 10 nM.
   15. The antibody of any one of embodiments 1-14, wherein the antibody is capable neutralizing Ara h 2
   16. An antibody that binds to the same epitope as the antibody of any one of embodiment 1-15.
   17. An antibody that competes for binding to Ara h 2 with the antibody of any one of embodiments 1-16.
   18. The antibody of embodiment 16 or 17, wherein whether the antibody binds to the same epitope or competes for binding to Ara h 2 is determined by an epitope binning assay.
   19. The antibody of any one of embodiments 1-18, wherein the antibody is monoclonal.
   20. The antibody of any one of embodiments 1-19, wherein the antibody is humanized.
   21. The antibody of any one of embodiments 1-20, wherein the antibody is an antibody fragment that binds Ara h 2.
   22. The antibody of embodiment 21, wherein the antibody fragment is selected from the group consisting of Fab, Fab′-SH, Fv, scFv, and (Fab′)₂ fragments.
   23. The antibody of any one of embodiments 1-20, wherein the antibody is a full-length antibody.
   24. The antibody of embodiment 23, wherein the antibody is an IgG antibody.
   25. The antibody of embodiment 24, wherein the IgG antibody is an IgG1 antibody.
   26. The antibody of embodiment 24, wherein the IgG antibody is an IgG4 antibody.
   27. The antibody of embodiment 26, wherein the IgG4 antibody comprises an S228P mutation (EU numbering).
   28. The antibody of any one of embodiments 1-27, wherein the antibody is a monospecific antibody.
   29. The antibody of any one of embodiments 1-27, wherein the antibody is a multispecific antibody.
   30. The antibody of embodiment 29, wherein the antibody is a bispecific antibody.
   31. A polynucleotide encoding an isolated antibody of any one of embodiments 1-30.
   32. A vector comprising the polynucleotide of embodiment 31.
   33. A host cell comprising the vector of embodiment 32.
   34. A method of producing the antibody of any one of embodiments 1-33, the method comprising culturing a host cell that comprises the vector of embodiment 32 and recovering the antibody.
   35. The method of embodiment 34, wherein the host cell is prokaryotic.
   36. The method of embodiment 35, wherein the host cell is Escherichia coli.
   37. The method of embodiment 34, wherein the host cell is eukaryotic.
   38. The method of embodiment 37, wherein the host cell is a 293 cell, a CHO cell, a yeast cell, or a plant cell.
   39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of embodiments 1-30.
   40. The method of embodiment 39, further comprising administering an effective amount of a second therapeutic agent useful for diminishing an allergic reaction an allergic reaction against, a nut or a portion thereof or an extract thereof, or to Ara h 2 protein.
   41. A method for detecting the presence of Ara h 2 neutralizing antibodies in a biological sample, comprising:
(a) providing a sample from a subject;
(b) contacting the sample with a mixture comprising:
   (i) the antibody of any one of embodiments 1-30 and
   (ii) Ara h 2, or a fragment thereof; and
(c) detecting the binding of antibodies present in the biological sample to Ara h 2, or a fragment thereof.
   42. The method of embodiment 41, where antibody binding is detected using tandem biolayer interferometry.
   43. The method of embodiments 41 or 42, where the antibody from step (b) is bound to a solid support.
   44. The method of embodiments 41 or 42, where Ara h 2, or a fragment thereof, is bound to a solid support.
   45. The method of embodiment 41, where antibody binding is detected using an enzyme-linked immunosorbent assay.

### T4:

1. An isolated antibody that binds to Ara h 2 (conglutin-7), or an antigen-binding fragment thereof, wherein the antibody comprises the following six CDRs:
(a) an CDR-H1 comprising the amino acid sequence of GDSISSYY (SEQ ID NO: 41);
(b) an CDR-H2 comprising the amino acid sequence of IFTSGST (SEQ ID NO: 42);
(c) an CDR-H3 comprising the amino acid sequence of ARDRRALSSDGNWYWYFDL (SEQ ID NO: 43)
(d) an CDR-L1 comprising the amino acid sequence of QTITRN (SEQ ID NO: 44);
(e) an CDR-L2 comprising the amino acid sequence of GAS (SEQ ID NO: 45); and
(f) an CDR-L3 comprising the amino acid sequence of QQSDNTPRT (SEQ ID NO: 46).
   2. The antibody of embodiment 1, wherein the antibody comprises
(a) a VH domain comprising an amino sequence having at least 90%, at least 95%, or at least 99% sequence identity to the amino acid sequence of SEQ ID NO: 47;
(b) a VL domain comprising an amino acid sequence having at least 90%, at least 95%, or at least 99% identity to the amino acid sequence of SEQ ID NO: 48; or (c) a VH domain as in (a) and a VL domain as in (b).
   3. The antibody of embodiment 1 or 2, further comprising the following VH domain FRs:
(a) an FR-H1 comprising the amino acid sequence of ESGPRLVKPSETLSLTCIVS (SEQ ID NO: 49);
(b) an FR-H2 comprising the amino acid sequence of WGWIRQPAGRGLEWIGR (SEQ ID NO: 50);
(c) an FR-H3 comprising the amino acid sequence of TYNPSLKSRVSMSVETSKNQFSLTLTSVTAADTAVYFC (SEQ ID NO: 51); and
(d) an FR-H4 comprising the amino acid sequence of WGRGTLVAVSS (SEQ ID NO: 52).
   4. The antibody of any one of embodiments 1-3, wherein the VH domain comprises the amino acid sequence of SEQ ID NO: 47.
   5. The antibody of any one of embodiments 1-4, further comprising the following VL domain FRs:
(a) an FR-L1 comprising the amino acid sequence of TQSPSSLSASVGDRVTITCRAS (SEQ ID NO: 53);
(b) an FR-L2 comprising the amino acid sequence of LNWYQQKSGEAPKLLIY (SEQ ID NO: 54);
(c) an FR-L3 comprising the amino acid sequence of ILQSGVPSRFTGSGSGTDFTLTISNLQPEDFASYSC (SEQ ID NO: 55); and
(d) an FR-L4 comprising the amino acid sequence of GQGTKVEIK (SEQ ID NO: 56).
   6. The antibody of any one of embodiments 1-5, wherein the VL domain comprises the amino acid sequence of SEQ ID NO: 48.
   7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 47 and
(b) a VL domain comprising an amino acid sequence having at least 90%, at least 95%, or at least 99% sequence identity to the amino acid sequence of SEQ ID NO: 48.
   8. The antibody of embodiment 7, wherein the antibody comprises a VH domain comprising the amino acid sequence of SEQ ID NO: 47 and a VL domain comprising the amino acid sequence of SEQ ID NO: 48.
   9. The antibody of any one of embodiments 1-8, wherein the antibody comprises (a) a heavy chain comprising the amino acid sequence of SEQ ID NO: 57 and (b) a light chain comprising the amino acid sequence of SEQ ID NO: 58.
   10. The antibody of any one of embodiments 1-9, wherein the antibody binds Ara h 2 with a KD of about 50 nM or less.
   11. The method of embodiment 10, wherein the KD is measured by a surface plasmon resonance assay.
   12. The antibody of embodiment 11, wherein the antibody binds Ara h 2 with a KD of between about 0.1 pM and about 40 nM.
   13. The antibody of embodiment 12, wherein the antibody binds Ara h 2 with a KD of between about 120 pM and about 20 nM.
   14. The antibody of embodiment 13, wherein the antibody binds Ara h 2 with a KD of between about 120 pM and about 10 nM.
   15. The antibody of any one of embodiments 1-14, wherein the antibody is capable neutralizing Ara h 2
   16. An antibody that binds to the same epitope as the antibody of any one of embodiment 1-15.
   17. An antibody that competes for binding to Ara h 2 with the antibody of any one of embodiments 1-16.
   18. The antibody of embodiment 16 or 17, wherein whether the antibody binds to the same epitope or competes for binding to Ara h 2 is determined by an epitope binning assay.
   19. The antibody of any one of embodiments 1-18, wherein the antibody is monoclonal.
   20. The antibody of any one of embodiments 1-19, wherein the antibody is humanized.
   21. The antibody of any one of embodiments 1-20, wherein the antibody is an antibody fragment that binds Ara h 2.
   22. The antibody of embodiment 21, wherein the antibody fragment is selected from the group consisting of Fab, Fab′-SH, Fv, scFv, and (Fab′)₂ fragments.
   23. The antibody of any one of embodiments 1-20, wherein the antibody is a full-length antibody.
   24. The antibody of embodiment 23, wherein the antibody is an IgG antibody.
   25. The antibody of embodiment 24, wherein the IgG antibody is an IgG1 antibody.
   26. The antibody of embodiment 24, wherein the IgG antibody is an IgG4 antibody.
   27. The antibody of embodiment 26, wherein the IgG4 antibody comprises an S228P mutation (EU numbering).
   28. The antibody of any one of embodiments 1-27, wherein the antibody is a monospecific antibody.
   29. The antibody of any one of embodiments 1-27, wherein the antibody is a multispecific antibody.
   30. The antibody of embodiment 29, wherein the antibody is a bispecific antibody.
   31. A polynucleotide encoding an isolated antibody of any one of embodiments 1-30.
   32. A vector comprising the polynucleotide of embodiment 31.
   33. A host cell comprising the vector of embodiment 32.
   34. A method of producing the antibody of any one of embodiments 1-33, the method comprising culturing a host cell that comprises the vector of embodiment 32 and recovering the antibody.
   35. The method of embodiment 34, wherein the host cell is prokaryotic.
   36. The method of embodiment 35, wherein the host cell is Escherichia coli.
   37. The method of embodiment 34, wherein the host cell is eukaryotic.
   38. The method of embodiment 37, wherein the host cell is a 293 cell, a CHO cell, a yeast cell, or a plant cell.
   39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of embodiments 1-30.
   40. The method of embodiment 39, further comprising administering an effective amount of a second therapeutic agent useful for diminishing an allergic reaction an allergic reaction against, a nut or a portion thereof or an extract thereof, or to Ara h 2 protein.
   41. A method for detecting the presence of Ara h 2 neutralizing antibodies in a biological sample, comprising:
(a) providing a sample from a subject;
(b) contacting the sample with a mixture comprising:
   (i) the antibody of any one of embodiments 1-30 and
   (ii) Ara h 2, or a fragment thereof; and
(c) detecting the binding of antibodies present in the biological sample to Ara h 2, or a fragment thereof.
   42. The method of embodiment 41, where antibody binding is detected using tandem biolayer interferometry.
   43. The method of embodiments 41 or 42, where the antibody from step (b) is bound to a solid support.
   44. The method of embodiments 41 or 42, where Ara h 2, or a fragment thereof, is bound to a solid support.
   45. The method of embodiment 41, where antibody binding is detected using an enzyme-linked immunosorbent assay.

### T5:

1. An isolated antibody that binds to Ara h 2 (conglutin-7), or an antigen-binding fragment thereof, wherein the antibody comprises the following six CDRs:
(a) an CDR-H1 comprising the amino acid sequence of GGSMSSYY (SEQ ID NO: 61);
(b) an CDR-H2 comprising the amino acid sequence of IFTTGST (SEQ ID NO: 62);
(c) an CDR-H3 comprising the amino acid sequence of VRDRRGRSHDSNWYWYFDL (SEQ ID NO: 63);
(d) an CDR-L1 comprising the amino acid sequence of QTLSRN (SEQ ID NO: 64);
(e) an CDR-L2 comprising the amino acid sequence of GAS (SEQ ID NO: 65); and
(f) an CDR-L3 comprising the amino acid sequence of QQSDNTPRT (SEQ ID NO: 66).
   2. The antibody of embodiment 1, wherein the antibody comprises
(a) a VH domain comprising an amino sequence having at least 90%, at least 95%, or at least 99% sequence identity to the amino acid sequence of SEQ ID NO: 67;
(b) a VL domain comprising an amino acid sequence having at least 90%, at least 95%, or at least 99% identity to the amino acid sequence of SEQ ID NO: 68; or (c) a VH domain as in (a) and a VL domain as in (b).
   3. The antibody of embodiment 1 or 2, further comprising the following VH domain FRs:
(a) an FR-H1 comprising the amino acid sequence of QVQLQESGPGLVKPSETLSLTCTVS (SEQ ID NO: 69);
(b) an FR-H2 comprising the amino acid sequence of WGWIRQPAGRGLEWIGR (SEQ ID NO: 70);
(c) an FR-H3 comprising the amino acid sequence of IYNASLNSRVSMSVDTSKNQFSLKLTSVTAADTALYFC (SEQ ID NO: 71); and
(d) an FR-H4 comprising the amino acid sequence of WGRGTLVTVSS (SEQ ID NO: 72).
   4. The antibody of any one of embodiments 1-3, wherein the VH domain comprises the amino acid sequence of SEQ ID NO: 67.
   5. The antibody of any one of embodiments 1-4, further comprising the following VL domain FRs:
(a) an FR-L1 comprising the amino acid sequence of RVIITCRAS (SEQ ID NO: 73);
(b) an FR-L2 comprising the amino acid sequence of LNWYQQKPGEAPKLLIY (SEQ ID NO: 74);
(c) an FR-L3 comprising the amino acid sequence of TLQSGVPSRFTGSGSGTDFTLIISGLQPEDFATYYC (SEQ ID NO: 75); and
(d) an FR-L4 comprising the amino acid sequence of FGQGT (SEQ ID NO: 76).
   6. The antibody of any one of embodiments 1-5, wherein the VL domain comprises the amino acid sequence of SEQ ID NO: 68.
   7. An isolated antibody that binds to Ara h 2, or an antigen-binding fragment thereof, wherein the antibody comprises
(a) a VH domain comprising an amino acid sequence having at least 90%, at least 95%, or at least 99% sequence identity to the amino acid sequence of SEQ ID NO: 67 and
(b) a VL domain comprising an amino acid sequence having at least 90%, at least 95%, or at least 99% sequence identity to the amino acid sequence of SEQ ID NO: 68.
   8. The antibody of embodiment 7, wherein the antibody comprises a VH domain comprising the amino acid sequence of SEQ ID NO: 67 and a VL domain comprising the amino acid sequence of SEQ ID NO: 68.
   9. The antibody of any one of embodiments 1-8, wherein the antibody comprises (a) a heavy chain comprising the amino acid sequence of SEQ ID NO: 77 and (b) a light chain comprising the amino acid sequence of SEQ ID NO: 78.
   10. The antibody of any one of embodiments 1-9, wherein the antibody binds Ara h 2 with a KD of about 50 nM or less.
   11. The method of embodiment 10, wherein the KD is measured by a surface plasmon resonance assay.
   12. The antibody of embodiment 11, wherein the antibody binds Ara h 2 with a KD of between about 0.1 pM and about 40 nM.
   13. The antibody of embodiment 12, wherein the antibody binds Ara h 2 with a KD of between about 120 pM and about 20 nM.
   14. The antibody of embodiment 13, wherein the antibody binds Ara h 2 with a KD of between about 120 pM and about 10 nM.
   15. The antibody of any one of embodiments 1-14, wherein the antibody is capable neutralizing Ara h 2
   16. An antibody that binds to the same epitope as the antibody of any one of embodiment 1-15.
   17. An antibody that competes for binding to Ara h 2 with the antibody of any one of embodiments 1-16.
   18. The antibody of embodiment 16 or 17, wherein whether the antibody binds to the same epitope or competes for binding to Ara h 2 is determined by an epitope binning assay.
   19. The antibody of any one of embodiments 1-18, wherein the antibody is monoclonal.
   20. The antibody of any one of embodiments 1-19, wherein the antibody is humanized.
   21. The antibody of any one of embodiments 1-20, wherein the antibody is an antibody fragment that binds Ara h 2.
   22. The antibody of embodiment 21, wherein the antibody fragment is selected from the group consisting of Fab, Fab′-SH, Fv, scFv, and (Fab′)₂ fragments.
   23. The antibody of any one of embodiments 1-20, wherein the antibody is a full-length antibody.
   24. The antibody of embodiment 23, wherein the antibody is an IgG antibody.
   25. The antibody of embodiment 24, wherein the IgG antibody is an IgG1 antibody.
   26. The antibody of embodiment 24, wherein the IgG antibody is an IgG4 antibody.
   27. The antibody of embodiment 26, wherein the IgG4 antibody comprises an S228P mutation (EU numbering).
   28. The antibody of any one of embodiments 1-27, wherein the antibody is a monospecific antibody.
   29. The antibody of any one of embodiments 1-27, wherein the antibody is a multispecific antibody.
   30. The antibody of embodiment 29, wherein the antibody is a bispecific antibody.
   31. A polynucleotide encoding an isolated antibody of any one of embodiments 1-30.
   32. A vector comprising the polynucleotide of embodiment 31.
   33. A host cell comprising the vector of embodiment 32.
   34. A method of producing the antibody of any one of embodiments 1-33, the method comprising culturing a host cell that comprises the vector of embodiment 32 and recovering the antibody.
   35. The method of embodiment 34, wherein the host cell is prokaryotic.
   36. The method of embodiment 35, wherein the host cell is Escherichia coli.
   37. The method of embodiment 34, wherein the host cell is eukaryotic.
   38. The method of embodiment 37, wherein the host cell is a 293 cell, a CHO cell, a yeast cell, or a plant cell.
   39. A method for treating a patient who demonstrates a sensitivity to, or an allergic reaction against, a nut or a portion thereof or an extract thereof, or to Ara h 2 protein, or for treating at least one symptom or complication associated with a sensitivity to, or allergic reaction against a nut or a portion thereof or an extract thereof, or to Ara h 2, comprising administering an effective amount of one or more isolated antibodies or antigen-binding fragments thereof that bind specifically to Ara h 2, according to any of embodiments 1-30.
   40. The method of embodiment 39, further comprising administering an effective amount of a second therapeutic agent useful for diminishing an allergic reaction an allergic reaction against, a nut or a portion thereof or an extract thereof, or to Ara h 2 protein.
   41. A method for detecting the presence of Ara h 2 neutralizing antibodies in a biological sample, comprising:
(a) providing a sample from a subject;
(b) contacting the sample with a mixture comprising:
   (i) the antibody of any one of embodiments 1-30 and
   (ii) Ara h 2, or a fragment thereof; and
(c) detecting the binding of antibodies present in the biological sample to Ara h 2, or a fragment thereof.
   42. The method of embodiment 41, where antibody binding is detected using tandem biolayer interferometry.
   43. The method of embodiments 41 or 42, where the antibody from step (b) is bound to a solid support.
   44. The method of embodiments 41 or 42, where Ara h 2, or a fragment thereof, is bound to a solid support.
   45. The method of embodiment 41, where antibody binding is detected using an enzyme-linked immunosorbent assay.

### Compositions and Methods

1. A pharmaceutical composition comprising a therapeutically effective amount of one or more isolated T1, T3, T4, or T5 antibodies, or antigen-binding fragments thereof, or of any of the V5-V41 antibodies, or antigen-binding fragments thereof, together with one or more pharmaceutically acceptable excipients.
2. A pharmaceutical composition comprising a combination of T3 and T4 antibodies.
3. A method for treating a patient who demonstrates a sensitivity to a peanut allergen, an allergic reaction against a peanut allergen, or to Ara h 2 protein, or for treating at least one symptom or complication associated with a sensitivity to, or allergic reaction against a peanut allergen, an allergic reaction against a peanut allergen, or to Ara h 2 protein, comprising administering an effective amount of one or more isolated human monoclonal antibodies or antigen-binding fragments thereof that bind specifically to Ara h 2, according to embodiments describe above forT1 (also referred to as V1), T3 (also referred to as V2), T4 (also referred to as V3), or T5 (also referred to as V4) to a patient in need thereof, wherein the sensitivity to, or an allergic reaction against, to a peanut allergen, an allergic reaction against a peanut allergen, or to Ara h 2 protein, is lessened in severity and/or duration, or at least one symptom or complication associated with the sensitivity to, or allergic reaction against, to a peanut allergen, an allergic reaction against a peanut allergen, or to Ara h 2 protein is ameliorated, or that the frequency and/or duration of, or the severity of the sensitivity to or allergic reaction against, to a peanut allergen, an allergic reaction against a peanut allergen, or to Ara h 2 protein, is reduced following administration of one or more of the isolated human monoclonal antibodies or fragments thereof that bind specifically to Ara h 2.
4. The method of embodiment 3, further comprising administering an effective amount of a second therapeutic agent useful for diminishing an allergic reaction to a peanut allergen, an allergic reaction against a peanut allergen, or to Ara h 2 protein.
5. The method of embodiment 4, wherein the second therapeutic agent is selected from the group consisting of a corticosteroid, a bronchial dilator, an antihistamine, epinephrine, or a decongestant.
6. The method of embodiment 3, wherein the treatment results in a reduction in allergic rhinitis, allergic conjunctivitis, allergic asthma, or an anaphylactic response following exposure, direct or indirect, of the patient to a peanut allergen, an allergic reaction against a peanut allergen, or to Ara h 2 protein.

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claim

1-109. (canceled)

110. A method for treating a patient who demonstrates a sensitivity to, or an allergic reaction against, a nut or a portion thereof or an extract thereof, or to Ara h 2 (conglutin-7), or for treating at least one symptom or complication associated with a sensitivity to, or allergic reaction against, a nut or a portion thereof or an extract thereof, or to Ara h 2, comprising administering an effective amount of an isolated antibody that binds to Ara h 2, or an antigen-binding fragment thereof, wherein the antibody or antigen-binding fragment thereof comprises the following six CDRs:
(a) a CDR-H1 comprising the amino acid sequence of GFTFRDYS (SEQ ID NO: 21);
(b) a CDR-H2 comprising the amino acid sequence of IRFDGTTK (SEQ ID NO: 22);
(c) a CDR-H3 comprising the amino acid sequence of AKDNGWRAFDH (SEQ ID NO: 23);
(d) a CDR-L1 comprising the amino acid sequence of QSLLHRNGYIY (SEQ ID NO: 24);
(e) a CDR-L2 comprising the amino acid sequence of FVS (SEQ ID NO: 25); and
(f) a CDR-L3 comprising the amino acid sequence of MQALETPWT (SEQ ID NO: 26).

111. The method of claim 110, wherein the patient has a peanut allergy.

112. The method of claim 110, further comprising administering an effective amount of a second therapeutic agent useful for diminishing a sensitivity to, or an allergic reaction against, a nut or a portion thereof or an extract thereof, or to Ara h 2.

113. The method of claim 112, wherein the second therapeutic agent is selected from the group consisting of a corticosteroid, a bronchial dilator, an antihistamine, epinephrine, or a decongestant.

114. The method of claim 110, wherein the treatment results in a reduction in allergic rhinitis, allergic conjunctivitis, allergic asthma, or an anaphylactic response following exposure of the patient to, or an allergic reaction against, a nut or a portion thereof or an extract thereof, or to Ara h 2.

115. The method of claim 110, wherein the sensitivity to, or the allergic reaction against, a nut or a portion thereof or an extract thereof, or to Ara h 2, is lessened in severity and/or duration following administration of the antibody or antigen-binding fragment thereof.

116. The method of claim 110, wherein the at least one symptom or complication associated with a sensitivity to, or allergic reaction against, a nut or a portion thereof or an extract thereof, or to Ara h 2 is ameliorated following administration of the antibody or antigen-binding fragment thereof.

117. The method of claim 110, wherein the frequency and/or duration of the allergic reaction against a nut or a portion thereof or an extract thereof, or to Ara h 2 is reduced following administration of the antibody or antigen-binding fragment thereof.

118. A method for treating a patient who demonstrates a sensitivity to, or an allergic reaction against, a nut or a portion thereof or an extract thereof, or to Ara h 2 (conglutin-7), or for treating at least one symptom or complication associated with a sensitivity to, or allergic reaction against, a nut or a portion thereof or an extract thereof, or to Ara h 2, comprising administering an effective amount of an isolated antibody that binds to Ara h 2, or an antigen-binding fragment thereof, wherein the antibody or antigen-binding fragment thereof comprises the following six CDRs:
(a) a CDR-H1 comprising the amino acid sequence of GGSMSSYY (SEQ ID NO: 61);
(b) a CDR-H2 comprising the amino acid sequence of IFTTGST (SEQ ID NO: 62);
(c) a CDR-H3 comprising the amino acid sequence of VRDRRGRSHDSNWYWYFDL (SEQ ID NO: 63);
(d) a CDR-L1 comprising the amino acid sequence of QTLSRN (SEQ ID NO: 64);
(e) a CDR-L2 comprising the amino acid sequence of GAS (SEQ ID NO: 65); and
(f) a CDR-L3 comprising the amino acid sequence of QQSDNTPRT (SEQ ID NO: 66).

119. The method of claim 118, wherein the patient has a peanut allergy.

120. The method of claim 118, further comprising administering an effective amount of a second therapeutic agent useful for diminishing a sensitivity to, or an allergic reaction against, a nut or a portion thereof or an extract thereof, or to Ara h 2.

121. The method of claim 120, wherein the second therapeutic agent is selected from the group consisting of a corticosteroid, a bronchial dilator, an antihistamine, epinephrine, or a decongestant.

122. The method of claim 118, wherein the treatment results in a reduction in allergic rhinitis, allergic conjunctivitis, allergic asthma, or an anaphylactic response following exposure of the patient to, or an allergic reaction against, a nut or a portion thereof or an extract thereof, or to Ara h 2.

123. The method of claim 118, wherein the sensitivity to, or the allergic reaction against, a nut or a portion thereof or an extract thereof, or to Ara h 2, is lessened in severity and/or duration following administration of the antibody or antigen-binding fragment thereof.

124. The method of claim 118, wherein the at least one symptom or complication associated with a sensitivity to, or allergic reaction against, a nut or a portion thereof or an extract thereof, or to Ara h 2 is ameliorated following administration of the antibody or antigen-binding fragment thereof.

125. The method of claim 118, wherein the frequency and/or duration of the allergic reaction against a nut or a portion thereof or an extract thereof, or to Ara h 2 is reduced following administration of the antibody or antigen-binding fragment thereof.
